(19) United States
(12) Patent Application Publication
(10) Pub. No.: US 2025/0268752 A1
(43) Pub. Date: Aug. 28, 2025

(54) INTRAOCULAR SYSTEMS, DEVICES, KITS, AND METHODS

(71) Applicant: Sight Sciences, Inc., Menlo Park, CA (US)

(72) Inventors: Daniel O'KEEFFE, San Francisco, CA (US); Paul BADAWI, Atherton, CA (US); David Y. BADAWI, Glenview, IL (US); Jose GARCIA, Fremont, CA (US); Trevor HANNON, Castro Valley, CA (US)

(21) Appl. No.: 19/065,990

(22) Filed: Feb. 27, 2025

Related U.S. Application Data
(60) Provisional application No. 63/558,585, filed on Feb. 27, 2024.

(51) Int. Cl. A61F 9/007

(52) U.S. Cl. CPC A61F 9/00781

(57) ABSTRACT

Disclosed herein are systems, devices, kits, and associated methods for treating conditions of the eye by reducing intraocular pressure. The system may include an ocular implant and a delivery device. The ocular implant may be implantable within Schlemm's canal to restore or maintain at least partial patency of the canal without substantially interfering with transmural or transluminal fluid flow across the canal. The delivery device may include a cannula comprising a straight proximal portion and a curved distal portion, where the curved distal portion comprises a first curve in a first direction. The delivery device may also include a positioning tool positioned at least partially within the cannula and configured to advance the ocular implant through the cannula, where the positioning tool comprises a curved distal portion biased to form a second curve in a second, different direction.

### CROSS REFERENCE TO RELATED APPLICATIONS

This application claims priority to U.S. Provisional Patent Application Ser. No. 63/558,585 filed Feb. 27, 2024, the content of which is incorporated herein by reference in its entirety for all purposes.

### TECHNICAL FIELD

This invention relates generally to intraocular systems, devices, and kits for treating conditions of the eye, and associated methods for treating such conditions of the eye.

### BACKGROUND

Glaucoma is a potentially blinding disease that affects over 60 million people worldwide, or about 1-2% of the population. Typically, glaucoma is characterized by elevated intraocular pressure. Increased pressure in the eye can cause irreversible damage to the optic nerve which can lead to loss of vision and even progress to blindness if left untreated. Consistent reduction of intraocular pressure can slow down or stop progressive loss of vision associated with glaucoma.

Increased intraocular pressure is generally caused by sub-optimal efflux or drainage of fluid (aqueous humor) from the eye. Aqueous humor or fluid is a clear, colorless fluid that is continuously replenished in the eye. Aqueous humor is produced by the ciliary body, and then ultimately exits the eye primarily through the trabecular meshwork. The trabecular meshwork extends circumferentially around the eye at the anterior chamber angle, or drainage angle, which is formed at the intersection between the peripheral iris or iris root, the anterior sclera or scleral spur and the peripheral cornea. The trabecular meshwork feeds outwardly into Schlemm's canal, a narrow circumferential passageway generally surrounding the exterior border of the trabecular meshwork. Positioned around and radially extending from Schlemm's canal are aqueous veins or collector channels that receive drained fluid. The net drainage or efflux of aqueous humor can be reduced as a result of decreased facility of outflow, decreased outflow through the trabecular meshwork and canal of Schlemm drainage apparatus, increased episcleral venous pressure, or possibly, increased production of aqueous humor. Flow out of the eye can also be restricted by blockages or constriction in the trabecular meshwork and/or Schlemm's canal and its collector channels.

Glaucoma, pre-glaucoma, and ocular hypertension currently can be treated by reducing intraocular pressure using one or more modalities, including medication, incisional surgery, laser surgery, cryosurgery, and other forms of surgery. In general, medications or medical therapy are the first lines of therapy. If medical therapy is not sufficiently effective, more invasive surgical treatments may be used. For example, a standard incisional surgical procedure to reduce intraocular pressure is trabeculectomy, or filtration surgery. This procedure involves creating a new drainage site for aqueous humor. Instead of naturally draining through the trabecular meshwork, a new drainage pathway is created by removing a portion of sclera and trabecular meshwork at the drainage angle. This creates an opening or passage between the anterior chamber and the subconjunctival space that is drained by conjunctival blood vessels and lymphatics. The new opening may be covered with sclera and/or conjunctiva to create a new reservoir called a bleb into which aqueous humor can drain. However, traditional trabeculectomy procedures carry both short- and long-term risks. These risks include blockage of the surgically created opening through scarring or other mechanisms, hypotony or abnormally low intraocular pressure, expulsive hemorrhage, hyphema, intraocular infection or endophthalmitis, shallow anterior chamber angle, macular hypotony, choroidal exudation, suprachoroidal hemorrhage, and others.

One alternative is to implant a device in Schlemm's canal that maintains the patency of the canal or aids flow of aqueous humor from the anterior chamber into the canal. Schlemm's canal is narrow (e.g., approximately 50 microns to 250 microns in cross-sectional diameter) and circular. Therefore, it can be difficult or expensive to design and manufacture hollow tubular stents of appropriate dimensions for use in opening Schlemm's canal. In addition, hollow tubular stents can be prone to failure and collapse or occlusion over time, and hollow tubular stents incorporating thin walls are especially prone to failure. Further, the walls of tubular stents placed lengthwise along Schlemm's canal can have significant surface area contact with the trabecular meshwork and/or the collector channels, which can result in blockage of the meshwork or collector channels, substantially interfering with transmural flow across the walls of Schlemm's canal and into the eye's collector channels, and with transluminal flow across the lumen of Schlemm's canal, thereby preventing restoration of normal eye drainage.

Moreover, various stents, shunts, catheters, and procedures that have been devised to maintain the patency of Schlemm's canal employ an ab-externo (from the outside of the eye) approach to deliver an implant or catheter into Schlemm's canal. This method of placement is invasive and typically prolonged, requiring the creation of tissue flaps and deep dissections to access the canal. Additionally, it is very difficult for many surgeons to find and access Schlemm's canal from this external incisional approach due to the small size of Schlemm's canal, which can be even smaller when collapsed. A related non-implant procedure, ab-externo canaloplasty, involves making a deep scleral incision and flap, finding and unroofing Schlemm's canal, circumnavigating all 360 degrees of the canal with a catheter from the outside of the eye, and either employing viscoelastic, a circumferential tensioning suture, or both to help maintain patency of the canal. The long-term safety and efficacy of canaloplasty is very promising, but the procedure remains surgically challenging and invasive, and can take anywhere from forty-five minutes to two hours.

An alternative procedure is viscocanalostomy, which involves the injection of a viscoelastic solution into Schlemm's canal to dilate the canal and associated collector channels. Dilation of the canal and collector channels in this manner generally facilitates drainage of aqueous humor from the anterior chamber through the trabecular meshwork and Schlemm's canal, and out through the natural trabeculocanalicular outflow pathway. Viscocanalostomy is similar to canaloplasty in that both are invasive and ab-externo, but viscocanalostomy does not involve a suture and does not restore all 360 degrees of outflow facility. Some advantages of viscocanalostomy are that sudden drops in intraocular pressure, hyphema, hypotony, and flat anterior chambers may be avoided. The risk of cataract formation and infection may also be minimized because of reduced intraocular manipulation and the absence of full eye wall penetration, anterior chamber opening and shallowing, and iridectomy. Another advantage of viscocanalostomy is that the procedure restores the physiologic outflow pathway, thus avoiding the need for external filtration, and its associated short and long term risks, in the majority of eyes. This makes the success of the procedure partly independent of conjunctival or episcleral scarring, which is a leading cause of failure in traditional trabeculectomy procedures. Moreover, the absence of an elevated filtering bleb avoids related ocular discomfort and potentially devastating ocular infections, and the procedure can be carried out in any quadrant of the outflow pathway.

However, both ab-externo viscocanalostomy and canaloplasty procedures can be disruptive to ocular tissue because Schlemm's canal is accessed externally by making a deep incision into the sclera, creating a scleral flap, and un-roofing Schlemm's canal. In contrast, an ab-interno (from the inside of the eye) approach may be a less invasive because less tissue is disrupted to access Schlemm's canal. Thus, an ab-interno approach to Schlemm's canal offers the surgeon easier access to the canal and leads improved patient recovery and rehabilitation due to reduced risk of injury to the patient's eye, complications from the procedure, and patient morbidity.

Accordingly, there is a need for easily manufacturable, minimally invasive devices for effective, long-term reduction in intraocular pressure. Systems, methods, and kits incorporating such devices are also desirable. Further, it would be beneficial to have systems, devices, kits, and methods that easily and atraumatically provide access to Schlemm's canal using an ab-interno approach for the delivery of tools (e.g., implants) and fluid compositions.

### SUMMARY

Disclosed herein is a system for treating an ocular condition including an ocular implant and a delivery device. The delivery device includes a cannula and a positioning tool. The cannula includes a straight proximal portion and a curved distal portion, where the curved distal portion includes a first curve in a first direction. The positioning tool is positioned at least partially within the cannula and configured to advance the ocular implant through the cannula, where the positioning tool includes a curved distal portion biased to form a second curve in a second, different direction. In some variations, when positioning tool is positioned in the straight proximal portion of the cannula, the curved distal portion of the positioning tool may have the second curve in the second direction. The second direction may be opposite the first direction. In some variations, the cannula may include a top cross-sectional arc and a bottom cross-sectional arc, and at least a portion of the curved distal portion of the positioning tool may be configured to contact an inner surface of the top cross-sectional arc of the cannula during at least a portion of delivery of the ocular implant into Schlemm's canal. In some variations, the cannula may have a plane longitudinally bisecting the straight proximal portion, and the curved distal portion of the cannula may curve toward a first side of the plane while the curved distal portion of the positioning tool may curve away from the first side of the plane. Similarly, some variations, the cannula may have a YZ-plane longitudinally bisecting the straight proximal portion, and at least a portion of the curved distal portion of the cannula may be on a first side of the YZ-plane and at least a portion of the curved distal portion of the positioning tool may be on a second side of the YZ-plane. In some variations, the first curve may have a first radius of curvature (ROC) and the second curve may have a second, greater ROC. In some variations, first curve may be configured to facilitate tangential entry of the ocular implant into at least a portion of Schlemm's canal of an eye. In some variations, the cannula may have a blunt distal tip.

In some variations, the positioning tool may further include a skived portion configured to increase flexibility of the positioning tool. The skived portion may be within the curved distal portion of the positioning tool. Moreover, the skived portion may extend longitudinally along a side of the positioning tool and face the first direction. In some variations, the skived portion may have a length of about 1 mm to about 8 mm. In some variations, the skived portion may have a depth of about 50 μm to about 150 μm.

In some variations, a distal portion of the positioning tool may be configured to interface with a proximal portion of the ocular implant. Further, the second curve of the positioning tool may be configured to cause at least a portion of the ocular implant to contact an interior surface of the cannula during advancement of the ocular implant through the cannula.

Another system for treating an ocular condition includes an ocular implant and a delivery device. The delivery device includes a cannula and a positioning tool. The cannula includes a curved distal portion, the curved distal portion having a proximal portion and a distal portion defining a first radius of curvature (ROC) therebetween. The positioning tool is positioned at least partially within the cannula and configured to advance the ocular implant through the cannula. Additionally, the positioning tool includes a curved distal portion with a proximal portion and a distal portion defining a second radius of curvature (ROC) therebetween, wherein the second ROC is greater than the first ROC. In some variations, the curved distal portion of the cannula may have a first curve with a first direction of curvature and the curved distal portion of the positioning tool may have a second curve with a second direction of curvature counter to the first direction of curvature. Moreover, the curved distal portion of the positioning tool may be biased to form the second curve.

Another system for treating an ocular condition includes an ocular implant and a delivery device. The delivery device includes a cannula having a top cross-sectional arc and a bottom cross-sectional arc. The positioning tool is positioned at least partially within the cannula and includes a curved distal portion, and the positioning tool is configured to advance the ocular implant through the cannula. Additionally, at least a portion of the curved distal portion of the positioning tool is configured to contact an inner surface of the top cross-sectional arc of the cannula during at least a portion of a procedure for delivering the ocular implant into Schlemm's canal. In some variations, the cannula may further include a curved distal portion with a first curve in a first direction, and the curved distal portion of the positioning tool may be biased to form a second curve in a second, different direction.

Also disclosed herein is an ocular implant including a body implantable circumferentially within at least a portion of Schlemm's canal. The body includes a twisted portion configured to restore or maintain at least partial patency of at least the portion of Schlemm's canal, a proximal portion extending from the twisted portion and configured to releasably couple to a delivery device, and a distal portion extending from the twisted portion, where the distal portion is non-twisted. The distal portion may be straight or folded. In some variations, the twisted portion of the body may include about 3 to about 12 twists. In some variations, the body may have a length of about 3 mm to about 20 mm. In some variations, the proximal and distal portions of the body may be integrally formed with the twisted portion of the body.

The twisted portion may include a first twisted segment and a second twisted segment, where the first twisted segment is coupled to the proximal portion of the body and the second twisted segment is positioned between the first twisted segment and the distal portion of the body. In some variations, first twisted segment may include a shorter length than the second twisted segment. In some variations, the first twisted segment may have more twists than the second twisted segment. In some variations, the first twisted segment may have a larger diameter than the second twisted segment. In some variations, the first twisted segment may have a smaller radius of curvature (ROC) than the second twisted segment. In some variations, an ROC of the first twisted segment and the proximal portion may be greater than the ROC of the second twisted segment.

In some variations, the proximal portion may include a hook configured to interface with the delivery device. The hook may be configured to interface with an opening extending transversely through a distal portion of a positioning tool of the delivery device. Alternatively, the hook may be configured to interface with a projection on a surface of a positioning tool of the delivery device. In some variations, the proximal portion may be flat and may include a tube coupled thereto, and the tube may be configured to interface with the delivery device. The tube may be configured to interface with a recess of a distal portion of a positioning tool of the delivery device. Alternatively, the tube may be configured to interface with a hook of a distal portion of a positioning tool of the delivery device. In some variations, the tube may include one or more fenestrations configured to facilitate aqueous flow through at least a portion of Schlemm's canal.

In some variations, the proximal portion may be configured to automatically release from the delivery device during delivery of the ocular implant to Schlemm's canal. The proximal portion may be configured to automatically release from a distal portion of a positioning tool of the delivery device when the distal portion of the positioning tool is advanced out of a cannula of the delivery device.

Also disclosed herein is a device for delivering an implant to an eye, including a handle, a drive assembly at least partially contained within the handle and including a linear gear, a lock releasably coupled to the handle and the linear gear, a cannula coupled to the handle, and a positioning tool slidably positioned within the cannula and coupled to the linear gear, where the positioning tool is configured to position the implant at least partially within Schlemm's canal of the eye. In some variations, a length of the linear gear may be about 0.25 times a length of the handle. In some variations, the drive assembly may be configured to actuate the positioning tool to advance the implant at least partially into Schlemm's canal of the eye.

In some variations, the lock may include a projection extending transversely through the handle. The projection may be configured to releasably engage a proximal portion of the linear gear. The proximal portion may include a recess and the projection may be configured to be translated relative to the recess to releasably engage the linear gear. In some variations, the lock may further include an exterior portion configured to at least partially surround an exterior surface of the handle, where the projection may be configured to be translated via user manipulation of the exterior portion of the lock.

Another device for delivering an implant to an eye includes a handle including a drive assembly at least partially contained therein, a cannula coupled to the handle, and a positioning tool within the cannula and coupled to the linear gear. The drive assembly is configured to translate rotational motion to linear motion and includes a linear gear and a rotatable wheel. The positioning tool is configured to position an implant at least partially within Schlemm's canal of the eye, and the device is configured to at least partially advance a proximal end of the implant out of the cannula when the rotatable wheel is rotated about 200 degrees to about 340 degrees. The rotatable wheel may be configured to be rotated by a finger of a user, and may be configured to actuate the linear gear to advance the positioning tool through the cannula. In some variations, the rotatable wheel may be a first rotatable wheel, the device may further include a second rotatable wheel. In such cases, the first rotatable wheel may be positioned at least partially within a top portion of the handle, and the second rotatable wheel may be positioned at least partially within a bottom portion of the handle.

Also disclosed herein is method for reducing intraocular pressure of an eye, including advancing a distal portion of a cannula of an ocular implant delivery device to Schlemm's canal via an ab interno approach, and delivering an ocular implant at least partially into Schlemm's canal by advancing a distal portion of the positioning tool out of the cannula. The delivery device includes a positioning tool, and the cannula includes a curved distal portion with a first curve in a first direction and the positioning tool includes a curved distal portion with a second curve that is biased in a second, different direction. In some variations, delivering the ocular implant may include automatically releasing a proximal portion of the ocular implant from a distal portion of the positioning tool. Additionally, the ocular implant may include a hook releasably couplable to an opening of the positioning tool. In some variations, delivering the ocular implant may include advancing the ocular implant within the cannula with the positioning tool, where at least a portion of the curved distal portion of the positioning tool may contact an inner surface of a top cross-sectional arc of the cannula during at least a portion of the advancement. In some variations, the second direction of the second curve may be opposite the first direction of the first curve. Further, in some variations, the first curve may include a first radius of curvature (ROC) and the second curve may include a second, greater ROC.

Also disclosed herein is a kit for treating ocular conditions, including an ocular implant delivery device and a fluid delivery device. The ocular implant delivery device includes a cannula with a top cross-sectional arc and a bottom cross-sectional arc and a positioning tool positioned at least partially within the cannula and having a curved distal portion, where the positioning tool is configured to advance the ocular implant through the cannula, and where at least a portion of the curved distal portion of the positioning tool is configured to contact an inner surface of the top cross-sectional arc of the cannula during at least a portion of a procedure for delivering the ocular implant into Schlemm's canal. The fluid delivery device includes a handle, a fluid reservoir positioned at least partially within the handle, and an elongate member fluidly coupled to the fluid reservoir for delivering fluid to Schlemm's canal. In some variations, the cannula may further include a curved distal portion with a first curve in a first direction, and the positioning tool may include a curved distal portion biased to form a second curve in a second, different direction.

Another kit for treating ocular conditions includes an ocular implant delivery device and a fluid delivery device. The ocular implant delivery device includes a cannula with a straight proximal portion and a curved distal portion, the curved distal portion having a first curve in a first direction, and a positioning tool positioned at least partially within the cannula and configured to advance the intraocular implant through the cannula, the positioning tool including a curved distal portion biased to form a second curve in a second, different direction. The fluid delivery device includes a handle, a fluid reservoir positioned at least partially within the handle, and an elongate member fluidly coupled to the fluid reservoir for delivering fluid to Schlemm's canal. In some variations, the second direction may be opposite the first direction. In some variations, the cannula may include a top cross-sectional arc and a bottom cross-sectional arc, and at least a portion of the curved distal portion of the positioning tool may be configured to contact an inner surface of the top cross-sectional arc of the cannula during at least a portion of delivery of the ocular implant into Schlemm's canal. In some variations, the cannula may have a top cross-sectional arc and a bottom cross-sectional arc, and at least a portion of the curved distal portion of the positioning tool may be configured to contact an inner surface of the top cross-sectional arc of the cannula during at least a portion of delivery of the ocular implant into Schlemm's canal. In some variations, the cannula may further include a plane longitudinally bisecting the straight proximal portion, and the curved distal portion of the cannula may curve toward a first side of the plane while the curved distal portion of the positioning tool may curve away from the first side of the plane. Further, in some variations, the first curve may have a first radius of curvature (ROC) and the second curve may have a second, greater ROC. Moreover, the fluid delivery device may further include a connector releasably coupled to the fluid reservoir in the handle. The connector may be configured to receive an external fluid device to transfer fluid into the fluid reservoir and to be rotated to be released from the handle with the external fluid device coupled thereto. In some variations, the elongate member may be slidably positioned in the cannula.

### BRIEF DESCRIPTION OF THE DRAWINGS

FIG. 1 shows a stylized, cross-sectional view of the eye and some of the structures involved in the flow of aqueous humor out of the eye.

FIGS. 2A-2B depict a perspective view of an exemplary ocular implant. FIG. 2C shows a stylized, cross-sectional view of Schlemm's canal with a variation of an ocular implant situated therein. FIGS. 2D-2F depict perspective views of exemplary proximal portions of an ocular implant.

FIG. 3 depicts a perspective view of another exemplary proximal portion of an ocular implant.

FIG. 4A depicts a side perspective view of another exemplary proximal portion of an ocular implant. FIG. 4B depicts a top perspective view of the exemplary proximal portion depicted in FIG. 4A.

FIG. 5A depicts a perspective view of an exemplary implant delivery device. FIGS. 5B and 5C depict cross-sectional and exploded views, respectively, of the exemplary delivery device depicted in FIG. 5A. FIG. 5D depicts a front view of a distal portion of a handle of the exemplary delivery device depicted in FIGS. 5A-5C.

FIG. 6 depicts a perspective view of an exemplary linear gear of an implant delivery device.

FIG. 7A depicts a cross-sectional view of an exemplary distal portion of a handle of an implant delivery device. FIG. 7B depicts a perspective view of the exemplary distal portion of the handle of the delivery device depicted in FIG. 7A.

FIG. 8 depicts a cross-sectional side view of an exemplary cannula of an implant delivery device with an ocular implant and positioning tool positioned therein.

FIG. 9 shows a stylized, perspective view of an exemplary cannula of an implant delivery device.

FIG. 10 shows a stylized, cross-sectional front view of an exemplary cannula of an implant delivery device.

FIG. 11A depicts a side view of an exemplary positioning tool of an implant delivery device. FIG. 11B depicts a side view of another exemplary variation of a positioning tool of an implant delivery device.

FIG. 12A depicts a cross-sectional side view of a cannula housing an exemplary positioning tool engaged with an exemplary ocular implant therein. FIG. 12B depicts a side view of a cannula housing an exemplary positioning tool engaged with an exemplary ocular implant outside of the distal exit of the cannula.

FIG. 13 depicts a cross-sectional side view of a cannula housing another exemplary variation of a positioning tool engaged with an exemplary ocular implant outside of the distal exit of the cannula.

FIG. 14 depicts a side view of a cannula housing another exemplary positioning tool engaged with an exemplary ocular implant outside of the distal exit of the cannula.

FIG. 15A depicts a bottom view of a cannula housing another exemplary positioning tool engaged with an exemplary ocular implant outside of the distal exit of the cannula. FIG. 15B depicts a side view of the cannula housing the exemplary positioning tool depicted in FIG. 15A.

FIGS. 16A-16B depict perspective views of exemplary fluid delivery devices.

FIG. 17 is a flow chart of an exemplary method for reducing intraocular pressure using one or more procedures described herein.

FIG. 18 is a flow chart of another exemplary method for reducing intraocular pressure using an ocular implant.

FIG. 19A depicts side views of a series of positions of an exemplary ocular implant relative to a cannula during an implantation procedure. FIGS. 19B-19D depict cross-sectional side views of a cannula housing an exemplary positioning tool and implant at an initial storage configuration of the exemplary system. FIGS. 19E-19G depict cross-sectional side views of a cannula housing an exemplary positioning tool and implant at a mid-deployment configuration of the exemplary system. FIGS. 19H and 19I depict a side view and a cross-sectional side view, respectively, of a cannula housing an exemplary positioning tool and implant at a deployed configuration of the exemplary system. FIGS. 19J and 19K also depict cross-sectional side views of the deployed configuration of the exemplary system shown in FIGS. 19H-19I.

FIG. 20 shows a stylized, top view of an exemplary variation of a distal portion of a positioning tool.

FIG. 21 depicts a cross-sectional view of an exemplary fluid delivery device.

### DETAILED DESCRIPTION

Described herein are systems, devices, kits, and methods for accessing Schlemm's canal and for delivering an implant (e.g., an ocular implant or support) to Schlemm's canal to reduce intraocular pressure and thereby treat conditions of the eye. The implant or support may be configured to restore or maintain at least partial patency (e.g., full patency) of Schlemm's canal so that at least a portion of the canal is unobstructed. The systems, devices, kits and methods may operate to keep Schlemm's canal from collapsing while not substantially interfering with the eye's natural drainage mechanism for aqueous humor, which includes transmural, transluminal and circumferential or longitudinal fluid flow into, across, around and out of Schlemm's canal.

The devices described herein may be implants that are generally capable of long-term maximization of the patency of Schlemm's canal while having minimal contact with the inner wall of the canal and the collector channel-based outer wall of the canal. Thus, the implants may exhibit minimal or no interference with transmural flow (across the inner and outer walls of Schlemm's canal—i.e., into and out of Schlemm's canal), transluminal flow (across Schlemm's canal), and/or circumferential or longitudinal flow (along Schlemm's canal). That is, when the implant is placed within Schlemm's canal, it will generally be configured to maintain the patency of Schlemm's canal without substantially interfering with transmural fluid flow across the canal. This may restore, enable, or enhance normal physiologic efflux of aqueous humor through the trabeculocanalicular tissues. As used herein, “without substantially interfering with transmural fluid flow” means that the implants do not significantly block either fluid outflow across and from the trabecular meshwork or fluid outflow to and through the collector channels.

As will be described herein, the implants may generally include a twisted body that traverses a central core of Schlemm's canal, the twisted body having a least one fenestration. In some cases, the devices (e.g., implants) may be implantable in Schlemm's canal with minimal trauma to the eye. Further, in some cases, the devices may also be coated with a drug useful for treating an ocular disorder (e.g., hypertension, glaucoma, or pre-glaucoma, infection, or scarring, neovascularization, fibrosis, or inflammation postoperatively). Additional exemplary implants (e.g., ocular implants, ocular supports, supports) that may be used with the systems, kits, and/or methods described herein are disclosed in U.S. Pat. No. 7,909,789 issued Mar. 22, 2011, U.S. Pat. No. 8,529,622 issued Sep. 10, 2013, and U.S. Pat. No. 8,894,603 issued Nov. 25, 2014, the contents of each of which are hereby incorporated by reference herein in their entirety.

To aid delivery of the implant, the systems, devices, kits, and methods may additionally be used to deliver a fluid composition (e.g., a fluid) to Schlemm's canal and/or to tear the trabecular meshwork. As used herein, the term “disrupting” refers to the delivery of a volume of fluid or a system component that alters the tissue in a manner that improves flow through the trabeculocanalicular outflow pathway. Examples of tissue disruption include, but are not limited to, dilation of Schlemm's canal, dilation of collector channels, increasing the porosity of the trabecular meshwork, stretching the trabecular meshwork, forming microtears or perforations in juxtacanalicular tissue, removing septae from Schlemm's canal, cutting, tearing, or removal of trabeculocanalicular tissues, or a combination thereof.

The delivery devices (e.g., implant delivery devices, fluid delivery devices) described herein may generally be configured for single-handed manipulation and for control by a single user and include one or more features useful for easily accessing Schlemm's canal with minimal trauma. Once access to the canal has been obtained, the delivery devices may deliver a fluid composition and/or tear the trabecular meshwork (e.g., the juxtacanalicular trabecular meshwork), and/or the delivery devices may be used to position an implant at least partially within Schlemm's canal. For example, the kits herein may generally include a first delivery device and a second, separate delivery device. The first delivery device may be an implant delivery device for delivering the implant to Schlemm's canal and the second delivery device may be a fluid delivery device for disrupting trabeculocanicular tissues, such as, for example, by delivering a fluid composition to Schlemm's canal and/or stretching the trabecular meshwork, forming microtears or perforations in juxtacanalicular tissue, removing septae from Schlemm's canal, cutting, tearing, or removal of trabeculocanalicular tissues. In some variations, the second delivery device may deliver fluid to aid delivery of the implant to the canal. Additional exemplary delivery devices (e.g., fluid delivery devices) that may be used with the systems, kits, and/or methods described herein are disclosed in U.S. patent application Ser. No. 18/454,585 filed Aug. 23, 2024, and U.S. application filed on Feb. 27, 2025 titled “OCULAR DELIVERY SYSTEM AND METHODS OF USE” to Needleman et al., having an attorney docket number of SGHT-015/01US 328518-2190, the contents of each of which are hereby incorporated by reference herein in their entirety.

The methods described herein may generally include advancing an implant delivery device through the anterior chamber (e.g., through at least a portion of the anterior chamber) and advancing an implant through the implant delivery device. The implant may be advanced through a cannula of the delivery device via a positioning tool that is movable within the canula. Additionally, the methods may include exposing the positioning tool (e.g., exposing at least a portion of a distal portion of the positioning tool from a distal portion of the cannula) to place the implant at least partially within Schlemm's canal (e.g., entirely within the canal, majority within the canal, etc.) and releasing the implant from the implant delivery device. In some variations, the implant may be released from the implant delivery device by retracting the implant delivery device to release the implant from the positioning tool of the implant delivery device. In some variations, the methods described herein may include implanting an implant completely or partially into Schlemm's canal in conjunction with delivering a fluid composition into the canal and/or otherwise disrupting tissue (e.g., tearing the trabecular meshwork).

Further, the systems, devices, kits, and methods herein may be used for a procedure with an ab-interno approach that minimizes an amount of unwanted ocular tissue disrupted and decreases procedure time for delivering tools (e.g., implants) and/or compositions into Schlemm's canal compared to conventional ab-externo approaches. Thus, applying the systems, devices, kits, and methods herein may reduce a patient's risk of infection and/or injury during delivery of an implant and/or a fluid composition, and/or desired tissue disruption (altering the tissue in a manner that improves flow through the trabeculocanalicular outflow pathway) without compromising safety and precision of the delivery procedure.

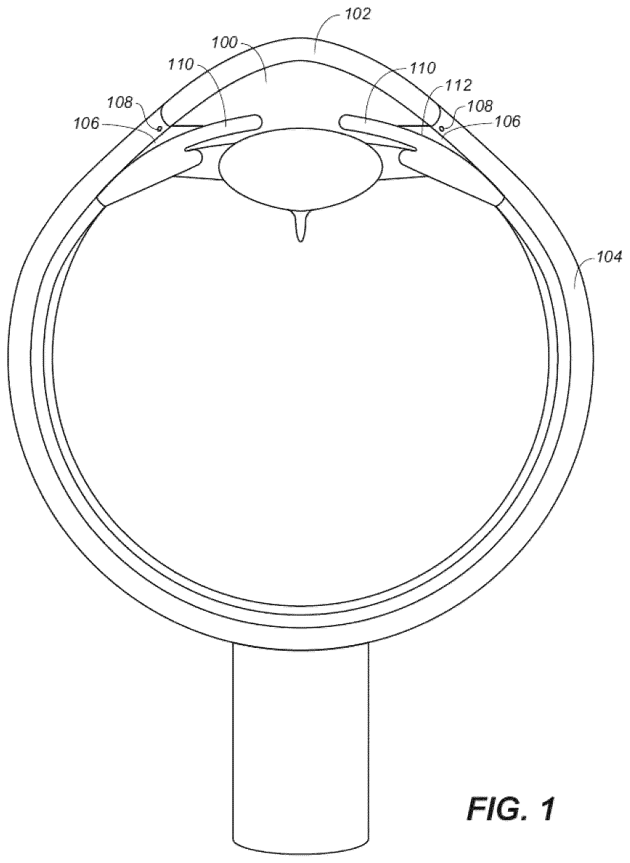

Several fundamental aspects of eye anatomy are described here with reference to FIG. 1 to provide a deeper understanding of the inventions described herein. FIG. 1 is a stylized depiction of a normal human eye. The anterior chamber 100 is shown as bounded on its anterior surface by the cornea 102. The cornea 102 is connected on its periphery to the sclera 104, which is a tough fibrous tissue forming the protective white shell of the eye. Trabecular meshwork 106 is located on the outer periphery of the anterior chamber 100. The trabecular meshwork 106 extends 360 degrees circumferentially around the anterior chamber 100. Located on the outer peripheral surface of the trabecular meshwork 106 is Schlemm's canal 108. Schlemm's canal 108 extends 360 degrees circumferentially around the meshwork 106. At the apex formed between the iris 110, meshwork 106, and sclera 104, is the anterior chamber angle 112.

### I. Systems and Devices

Generally, systems for treating an ocular condition or disorder (e.g., ocular treatment systems) described herein may include an implant delivery device having an implant (e.g., an ocular implant) positioned therein. The implant delivery device may be used to position the implant at least partially within Schlemm's canal. For example, a user may actuate a positioning tool of the implant delivery device via a drive assembly of the device to advance the implant at least partially into Schlemm's canal. As will be described in further detail herein, a curvature of a cannula of the delivery device may be in a first direction and a curvature of a positioning tool of the device may be in a second, different direction. The differing curvatures of the cannula and the positioning tool may configure the positioning tool to maintain contact with the implant as it advances the implant through the cannula, as well as passively or automatically release the implant from the positioning tool, and thus the implant delivery device, once a portion of positioning tool is advanced out of the cannula. Passive release of the implant from the positioning tool may constitute, for example, releasing the implant without changing a configuration of the delivery device, as will be described herein. Automatic release of the implant from the positioning tool may constitute, for example, releasing the implant without changing a position or configuration of the delivery device with respect to the implant.

### Implants

The implants described herein may generally include a twisted portion (e.g., coiled, winding/wound, spiraled, looped, etc.), a distal portion, and a proximal portion. The twisted portion of the implant may be configured to at least partially maintain patency of Schlemm's canal, and in some variations, may have a double helix configuration. The proximal portion of the implant may comprise the proximal end of the implant, and may be configured to releasably couple to an implant delivery device. For example, an implant may be maintained within the implant delivery device via the proximal portion of the implant. In some variations, when the proximal portion of the implant is advanced out of the implant delivery device, the implant may be released from the implant delivery device (e.g., automatically released) and/or the implant may be released from the implant delivery device upon retraction of the implant delivery device away from implant.

The implant may be configured to be circumferentially placed within at least a portion of Schlemm's canal to at least partially maintain patency of the portion of Schlemm's canal. The implant may be shaped to traverse a central core of Schlemm's canal, which refers to the region around the cross-sectional center of the canal in the interior space of the canal lumen (i.e., not including the periphery of the canal). Therefore, an implant that occupies at least a portion of a central core of Schlemm's canal may traverse at least a portion of the canal's lumen. In some cases, the implant may completely traverse the central core of Schlemm's canal. In some variations, the implants may be positioned entirely within Schlemm's canal (e.g., via the implant delivery devices described herein). In other variations, the implants may be implanted within the canal, but may extend partially beyond Schlemm's canal (e.g., into the trabecular meshwork and/or into the anterior chamber). The implants may contact at least one wall portion (e.g., two wall portions) of Schlemm's canal. As an example, implants described herein may contact first and second interior wall portions of Schlemm's canal, where the first interior wall portion is coincident with an outer peripheral boundary of the trabecular meshwork, and the second interior wall portion has collector channels extending therefrom. In some variations, the implant may have minimal surface area contact with the interior surface of Schlemm's canal by only tangentially touching the interior surface, and may be configured and positioned such that the majority of its mass is in the central core of Schlemm's canal.

The implant may not substantially interfere with transmural flow, and in many cases may allow about 0.1 microliter per minute to about 5 microliters per minute of aqueous outflux from the eye through the trabecular meshwork and collector channels. In some variations, the implants may reduce intraocular pressure by 1-40 mm Hg, such as, for example, by at least 2 mmHg, at least 4 mmHg, at least 6 mmHg, at least 10 mmHg, at least 20 mmHg, or within a range of about 8 mmHg and about 22 mmHg.

The implants herein, such as at least a portion thereof, may include one or more biocompatible materials, such as, for example, biocompatible polymers. Non-limiting examples of polymers that may be appropriate include acrylics, silicones, polymethylmethacrylate, polypropylene, and hydrogels. In some variations, the implant may include one or more biocompatible, biodegradable polymers. Such biodegradable polymers may include, for example, collagen, a collagen derivative, a poly(lactide); a poly(glycolide); a poly(lactide-co-glycolide); a poly(lactic acid); a poly(glycolic acid); a poly(lactide)/poly (ethylene glycol) copolymer; a poly(glycolide)/poly(ethylene glycol) copolymer; a poly(lactide-co-glycolide)/poly(ethylene glycol) copolymer; a poly(lactic acid)/poly(ethylene glycol) copolymer; a poly(glycolic acid)/poly(ethylene glycol) copolymer; a poly(lactic acid-co-glycolic acid)/poly(ethylene glycol) copolymer; a poly(caprolactone); a poly(caprolactone)/poly(ethylene glycol) copolymer; a polyorthoester; a poly(phosphazene); a poly(hydroXYbutyrate) or a copolymer including a poly(hydroXYbutyrate); a poly(lactide-co-caprolactone); a polycarbonate; a poly(esteramide); a polyanhydride; a poly(dioxanone); a poly(alkylene alkylate); a copolymer of polyethylene glycol and a polyorthoester; a biodegradable polyurethane; a poly(amino acid); a polyetherester; a polyacetal; a polycyanoacrylate; a poly(oXYethylene)/poly (oXYpropylene) copolymer; or a blend or copolymer thereof. In some variations, an implant may comprise modified scleral tissue. In some variations, the implant may comprise a suture (e.g., a modified suture).

Additionally, or alternatively, at least a portion of the implant (e.g., all of the implant, a proximal portion, a distal portion) may include one or more biocompatible metals, such as gold or titanium, and/or one or more biocompatible metal alloys, such as stainless steel or nickel-titanium alloys (e.g., Nitinol). In some variations, at least a portion of the implant may comprise one or more shape-memory materials. Suitable shape-memory materials include, but are not limited to, shape-memory polymers or shape-memory alloys, such as nickel-titanium alloys (e.g., Nitinol). In certain variations (e.g., certain variations in which a shape-memory material is used), the implant may have a compressed state prior to and during implantation into Schlemm's canal, and an expanded state following implantation (e.g., to open the canal). This may, for example, allow for relatively easy and/or efficient delivery and placement of the implant into Schlemm's canal.

The implants herein may be fabricated using any suitable technique, such as one or more of computer numerical control (CNC) machining, laser cutting, electrical discharge machining (EDM), powder metallurgy, investment casting, injection molding, compression molding, extrusion, additive manufacturing (3D printing), microfabrication techniques, tissue engineering, and the like. In some variations, at least a portion of the implant (e.g., the entire implant, the proximal portion, the distal portion) may be shape set during fabrication. The shape setting may be accomplished using any suitable means, such as, for example, a heat treatment (e.g., via salt bath, fluidized bed furnace, or the like). The heat treatment may be in the range of about 450 degrees Celsius to about 600 degrees Celsius, such as about 455 degrees to about 590 degrees, about 460 degrees to about 580 degrees, about 465 degrees to about 570 degrees, about 470 degrees to about 560 degrees, about 475 degrees to about 550 degrees, about 480 degrees to about 540 degrees, about 485 degrees to about 530 degrees, about 490 degrees to about 520 degrees, about 495 degrees to about 510 degrees, or about 500 degrees to about 505 degrees. In some variations, the implants may be fabricated as solid or semi-solid forms. For example, an entirety of the implant may be solid, or at least a portion of the implant may be solid. As another example, all or at least a portion of the implant may be hollow or porous. In some variations, the implant may comprise one or more fenestrations. Further, a surface of the implant may be smooth, rough, spiked, and/or fluted, and/or may have one or more other modifications. In certain variations, at least part of the implant may comprise a mesh.

In certain variations, the implant may include one or more active agents. For example, an implant may be coated or impregnated with an active agent. Alternatively, or additionally, an active agent may be dispersed within the implant (e.g., by filling a cavity within the implant and/or otherwise using an active agent-eluting material (e.g., a bioerodible active agent-eluting material). The release of an active agent may be controlled using a time-release (e.g., sustained-release) system (e.g., by embedding and/or encapsulating the active agent within a time-release (e.g., sustained-release) composition).

In the methods and systems described herein, the agent (e.g., therapeutic agent) may be any suitable agent. In some variations the therapeutic agent(s) may be glaucoma drugs such as prostaglandins, beta blockers, miotics, alpha adrenergic agonists, or carbonic anhydrase inhibitors. In some variations the therapeutic agent(s) may be anti-inflammatory drugs such as NSAIDs, corticosteroids, or other steroids. For example, steroids such as prednisolone, prednisone, cortisone, cortisol, triamcinolone, or shorter acting steroids may be employed. In some variations the therapeutic agent(s) may be antimetabolites such as 5-fluoruracil or mitomycin C. In some variations the therapeutic agent(s) may be drugs or antibodies that prevent neovascularization, such as bevacizumab, ranibizumab, and others. In some variations, exemplary active agents may include one or more of a prostaglandin, a prostaglandin analog (e.g., latanoprost), a beta blocker, an alpha-2 agonist, a calcium channel blocker, a carbonic anhydrase inhibitor, a growth factor, an antimetabolite, a chemotherapeutic agent, a steroid, a non-steroidal anti-inflammatory agent, an antagonist of a growth factor, or a combination thereof.

The implants herein may have a geometry, including one or more dimensions, configured for maintaining patency of at least a portion of Schlemm's canal. For example, the implants may have a width that is about equal to or less than a width or diameter of Schlemm's canal. Alternatively, the implants may have a width that is about equal to or greater than the width or diameter of Schlemm's canal. In some variations, an implant may have a width of about 100 μm to about 400 μm, such as about 125 μm to about 375 μm, about 150 μm to about 350 μm, about 175 μm to about 325 μm, about 200 μm to about 300 μm, about 210 μm to about 290 μm, about 220 μm to about 280 μm, about 230 μm to about 270 μm, or about 240 μm to about 260 μm. For example, the diameter of the implant may be about 100 μm, about 150 μm, about 200 μm, about 210 μm, about 220 μm, about 230 μm, about 240 μm, about 245 μm, about 250 μm, about 255 μm, about 260 μm, about 270 μm, about 280 μm, about 290 μm, or about 300 μm. Moreover, an implant may have a length of about 0.1 mm to about 40 mm, such as about 0.25 mm to about 35 mm, about 0.5 mm to about 30 mm, about 0.75 mm to about 25 mm, about 1 mm to about 20 mm, about 1.25 mm to about 15 mm, about 1.5 mm to about 10 mm, about 2 mm to about 9.75 mm, about 2.25 mm to about 9.5 mm, about 2.5 mm to about 9.25 mm, about 2.75 mm to about 9 mm, about 3 mm to about 8.75 mm, about 3.25 mm to about 8.5 mm, about 3.5 mm to about 8.25 mm, about 3.75 mm to about 8 mm, about 4 mm to about 7.75 mm, about 4.25 mm to about 7.5 mm, about 4.5 mm to about 7.25 mm, about 4.75 mm to about 7 mm, about 5 mm to about 6.75 mm, about 5.25 mm to about 6.5 mm, about 5.5 mm to about 6.25 mm, or about 5.75 mm to about 6 mm (including all values and sub-ranges therein). In some variations, the length of the implant may be about 5 mm to about 10 mm, such as about 6 mm to about 9 mm, about 7 mm to about 8 mm, or about 7.5 mm (including all values and sub-ranges therein).

Moreover, the implants herein may have a body that forms an arc (e.g., a bend or curve, which may be shape set) that configures it to occupy at least a portion of Schlemm's canal. The arc of an implant body may have an angular span of about 10 degrees to about 360 degrees. In some variations, the angular span may be about 20 degrees to about 300 degrees, about 45 degrees to about 225 degrees, about 90 degrees to about 180 degrees, or about 100 degrees to about 135 degrees, about 45 degrees (including all values and sub-ranges therein). In certain variations, the angular span may be about 90 degrees, about 135 degrees, or about 180 degrees.

The arc of the implant body may have an arc length corresponding to an arc length of a portion of Schlemm's canal. For example, the arc length may be about 2 mm to about 15 mm, such as about 3 mm to about 14 mm, about 4 mm to about 13 mm, about 5 mm to about 12 mm, about 6 mm to about 11 mm, about 6.5 mm to about 10.5 mm, about 7 mm to about 10 mm, about 7.25 mm to about 9.25 mm, about 7.5 mm to about 9.5 mm, about 8 mm to about 9 mm, or about 8.25 mm to about 8.75 mm. As another example, the arc length may be about 2 mm, about 2.5 mm, about 3 mm, about 3.5 mm, about 4 mm, about 4.5 mm, about 5 mm, about 5.5 mm, about 6 mm, about 6.25 mm, about 6.5 mm, about 6.75 mm, about 7 mm, about 7.25 mm, about 7.5 mm, about 7.75 mm, about 8 mm, about 8.25 mm, about 8.5 mm, about 8.75 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm. Put another way, the implant may be configured to occupy a fraction or percentage of the circumference of Schlemm's canal (360 degrees when intact) when positioned therein. For example, the implant may be configured to occupy about 5% to about 100%, about 10% to about 90%, about 15% to about 80%, about 20% to about 70%, about 25% to about 60%, about 30% to about 50%, or about 35% to about 40% of the circumference of the canal (including all values and sub-ranges therein).

Additionally, or alternatively, the arc of the implant body may define a radius of curvature that allows an implant to be easily positioned within the canal. For example, the radius of curvature (ROC) of the implant may be about 1 mm to about 11 mm, such as about 1.5 mm to about 10.5 mm, about 2 mm to about 10 mm, about 2.5 mm to about 9.5 mm, about 3 mm to about 9 mm, about 3.5 mm to about 8.5 mm, about 4 mm to about 8 mm, about 4.5 mm to about 7.5 mm, about 5 mm to about 7.25 mm, about 5.25 mm to about 7.125 mm, about 5.5 mm to about 7 mm, or about 6 mm to about 6.5 mm (including all values and sub-ranges therein). For example, the ROC of the implant may be about 1 mm, about 1.5 mm, 2 mm, about 2.5 mm, about 3 mm, about 3.5 mm, about 4 mm, about 4.5 mm, about 5 mm, about 5.25 mm, about 5.5 mm, about 5.75 mm, about 6 mm, about 6.25 mm, about 6.5 mm, about 6.75 mm, about 7 mm, about 7.5 mm, about 8 mm, about 8.5 mm, about 9 mm, about 9.5 mm, about 10 mm, about 10.5 mm, or about 11 mm. In general, the ROC of the implant 200 may be about equal to or less than an ROC of Schlemm's canal to enable the implant 200 to maintain at least partial patency of Schlemm's canal.

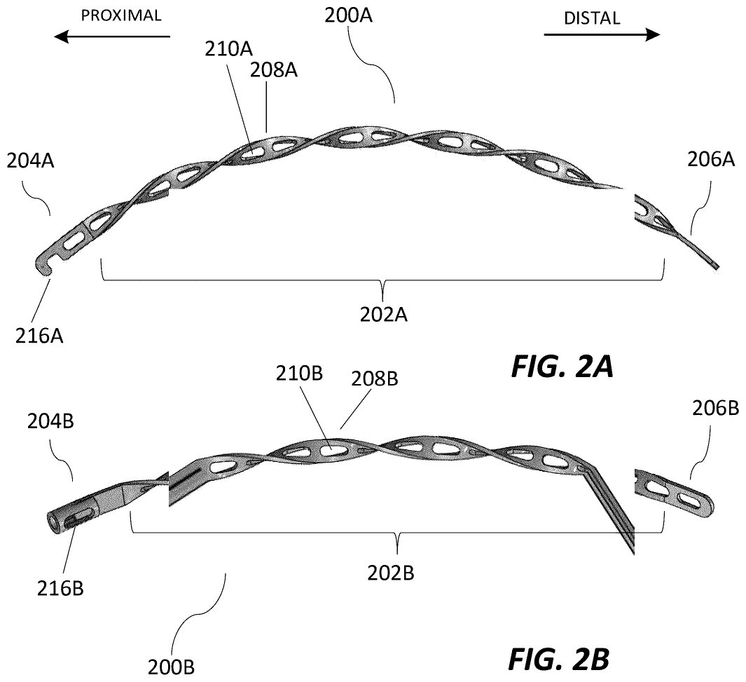

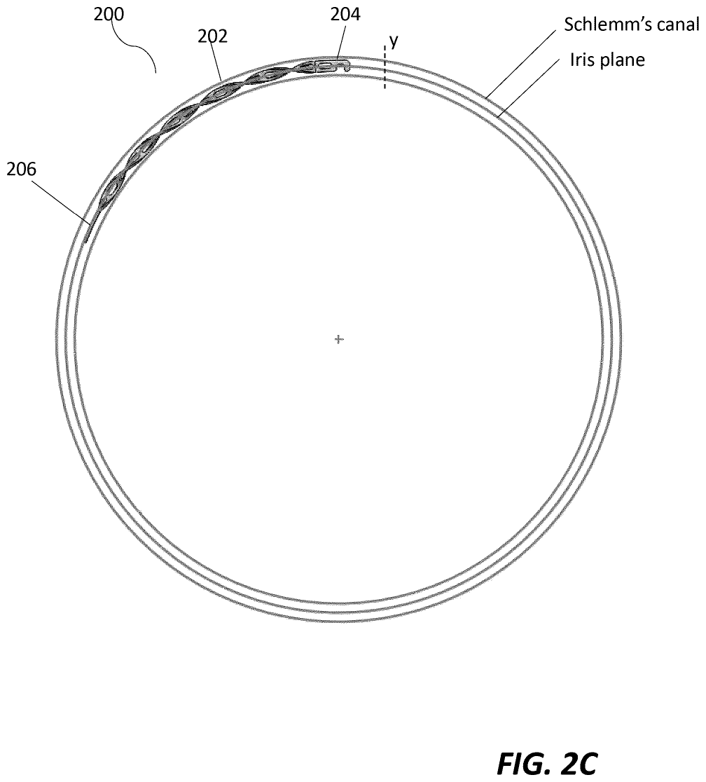

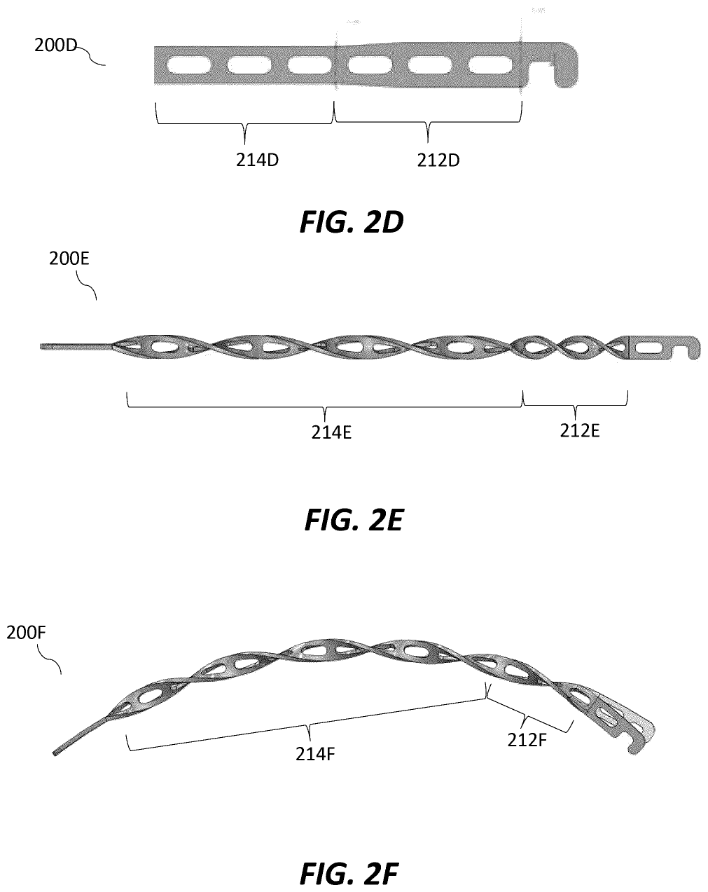

FIGS. 2A and 2B depict exemplary variations of implants 200A, 200B. The implants 200A, 200B may comprise an implant body that may include a central portion 202A, 202B, a proximal portion 204A, 204B, and a distal portion 206A, 206B. As shown, the implant bodies may have an arc extending from a proximal end of the proximal portions 204A, 204B to a distal end of the distal portions 206A, 206B. Accordingly, an angular span, arc length, and ROC of the implants 200A, 200B may be defined between the proximal portions 204A, 204B and distal portions 206A, 206B.

The central portion of the implant may be configured to keep Schlemm's canal at least partially open to transmural fluid flow. As shown in FIGS. 2A and 2B, the central portion 202A, 202B may include one or more twists 208A, 208B and/or one or more fenestrations 210A, 210B for allowing transluminal flow across the canal. Accordingly, the central portion 202A, 202B may be considered a twisted portion of the implant 200. A number of the twists 208A, 208B of the central portion 202A, 202B may be about 1 to about 20 twists, such as about 2 and about 19, about 3 to about 18, 4 to about 17, about 5 to about 16, about 6 to about 15, about 7 to about 14, about 8 to about 13, about 9 to about 12, or about 10 to about 11 twists (including all values and sub-ranges therein). In some variations, the number of twists 208A, 208B may be about 3 to about 12 twists, such as about 4 to about 11, about 5 and about 10, about 6 and about 9, or about 7 and about 8 twists (including all values and sub-ranges therein). As another example, in some variations, the number of twists 208A, 208B may be about 1, about 1.5, about 2, about 1.5, about 3, about 3.5, about 4, about 4.5, about 5, about 5.25, about 5.5, about 5.75, about 6, about 6.25, about 6.5, about 6.75, about 7, about 7.25, about 7.5, about 7.75, about 8, about 8.25, about 8.5, about 8.75, about 9, about 9.25, about 9.5, about 9.75, or about 10 (including all values and sub-ranges therein). A pitch (i.e., number of twists per unit length) of the central portion 202A, 202B may be about 0.2 twists/mm to about 1.5 twists/mm, such as about 0.225 twists/mm to about 1.475 twists/mm, about 0.25 twists/mm and about 1.45 twists/mm, about 0.275 twists/mm to about 1.425 twists/mm, about 0.3 twists/mm to about 1.4 twists/mm, about 0.325 twists/mm to about 1.375 twists/mm, or about 0.36 twists/mm to about 1.33 twists/mm (including all values and sub-ranges therein). For example, the pitch of the central portion 202A, 202B may be about 0.67 twists/mm.

The implant body may comprise one or more fenestrations to facilitate fluid flow through the implant body. As shown in FIGS. 2A and 2B, fenestrations 210A, 210B may generally be positioned in the central portions 202A, 202B of an implant body. However, in some variations, fenestrations may additionally or alternatively be positioned in proximal and/or distal portions of an implant body. For example, in some variations, a central portion of the implant body may comprise a plurality of fenestrations (e.g., 2, 3, 4, 5, 6, 7, 8, 9, 10, 11, 12, 13, 14, 15, 16, 17, 18, 19, 20, or more) and the proximal portion of the implant body may comprise a single fenestration or a plurality of fenestrations. In some variations, the central portion of the implant body may comprise a plurality of fenestrations (e.g., 2, 3, 4, 5, 6, 7, 8, 9, 10, 11, 12, 13, 14, 15, 16, 17, 18, 19, 20, or more) and a distal portion of the implant body may comprise a single fenestration or a plurality of fenestrations. Further, in some variations, the central portion of the implant body may comprise a plurality of fenestrations (e.g., 2, 3, 4, 5, 6, 7, 8, 9, 10, 11, 12, 13, 14, 15, 16, 17, 18, 19, 20, or more) and the proximal and distal portions of the implant body may each comprise one or more fenestrations (e.g., one fenestration or a plurality of fenestrations).

Each of the one or more fenestrations may be shaped and dimensioned (e.g., have a length, width, diameter, etc.) to allow for fluid flow therethrough. For example, each of the one or more fenestrations may have a length of about 100 μm to about 500 μm, such as about 150 μm and about 450 μm, about 200 μm to about 400 μm, about 250 μm to about 350 μm, about 275 μm to about 325 μm, about 280 μm to about 320 μm, about 290 μm to about 310 μm, or about 295 μm to about 305 μm (including all values and sub-ranges therein). Each of the one or more fenestrations may have a width of about 20 μm to about 500 μm, such as about 25 μm to about 450 μm, about 30 μm to about 400 μm, about 35 μm and about 350 μm, about 40 μm to about 300 μm, about 45 μm to about 250 μm, or about 50 μm to about 200 μm (including all values and sub-ranges therein). For example, as depicted in FIGS. 2A and 2B, a fenestration 210A, 210B may have a length of about 200 μm to about 400 μm (e.g., about 300 μm long) and a width of about 50 μm to about 200 μm (e.g., about 120 μm wide). The fenestrations may have any shape suitable for facilitating fluid flow through the implant body, such as, for example, square, rectangular, circular, ovular, triangular, diamond, hexagonal, pentagonal, or the like. In variations in which circular fenestrations are utilized, a diameter of the fenestrations may be any of the lengths or widths described above. In some variations, some or all of the fenestrations 210A, 210B may have the same dimensions and/or shapes. Additionally, or alternatively, some or all of the fenestrations 210A, 210B may have one or more unique dimensions (e.g., a unique length and/or a unique width) or shapes. In some variations, the fenestrations may include a first set of one or more (e.g., 2, 3, 4, 5, or more) fenestrations having first dimensions and/or shapes, and a second set of one or more (e.g., 2, 3, 4, 5, or more) fenestrations having second, different dimensions and/or shapes. Any combination of fenestrations having similar or unique dimensions and/or shapes may be utilized.

The implant body may have features that enable the implant to interface with a positioning tool and/or a cannula of an implant delivery device during an implantation procedure. For example, a varying diameter, pitch, and/or ROC of the central portion of the implant body may increase or strengthen a coupling or interference between the implant and the positioning tool and/or the implant and the cannula (e.g., an interior surface of the cannula) during implantation. In some variations, less than an entirety of the implant body, such as a segment of the central portion of the implant body, may include one or more such features. For example, the central portion may have a plurality of segments (e.g., two segments), and at least one of the plurality of segments may have one or more unique features compared to the remaining segments of the plurality, such as one or more of a unique diameter, a unique twist pitch, and a unique ROC. Generally, each of the plurality of segments may be integrally formed. In some variations, a first (e.g., proximal) segment of the central portion may have a first diameter, number of twists, and/or ROC, and a second (e.g., distal) segment of the central portion may have a second, different diameter, number of twists, and/or ROC. Each of first diameter, number of twists, and/or ROC may be larger or smaller than the second diameter, number of twists, and/or ROC.

In some variations, a first segment of the central portion may extend distally from the proximal portion of the implant body and a second segment may be positioned between the first segment and a distal portion of the implant body (e.g., extending distally from the first segment). In some variations, the first segment may additionally include the proximal portion, and/or the second segment may additionally include the distal portion. In some variations, the first segment may only include the proximal portion, and/or the second segment may only include the distal portion. Moreover, a length of the first segment may be less than a length of the second segment. For example, the length of the first segment (with or without the proximal portion) may be about 0.5 mm to about 5 mm of a total length of the implant, such as about 1 mm to about 4 mm, about 1.5 mm to about 3 mm, about 2 mm to about 2.5 mm (including all values and sub-ranges therein). In some variations, the length of the first segment (with or without the proximal portion) may be about 1 mm, about 1.5 mm, about 2 mm, about 2.5 mm, about 3 mm, about 3.5 mm, about 4 mm, about 4.5 mm, or about 5 mm. In some variations, the first and second segments may have substantially equal lengths.

FIGS. 2D-2F show three variations of an implant 200D, 200E, 200F where a first segment 212D, 212E, 212F of an implant 200D, 200D, 200F differs from a second segment 214D, 214E, 214F of the implant 200D, 200D, 200. The first segment 212D, 212E, 212F may have one or more of a larger width (FIG. 2D), a greater twist pitch (FIG. 2E), and a smaller ROC than the second segment 214D, 214E, 214F, or vice versa. For example, in variations in which the second segment has a greater width than the first segment, a width of the first segment (e.g., first segment 212D) may be about 10-100 μm greater than a width of the second segment (e.g., second segment 214D). For example, a first width of the first segment 212D may be about 215 μm to about 400 μm, and a second with of the second segment 214D may be about 200 μm to about 275 μm (including all values and sub-ranges therein). In some variations, the first width may be about 260 μm, about 270 μm, about 280 μm, about 290 μm, about 300 μm, about 310 μm, about 320 μm, about 330 μm, about 340 μm, or about 350 μm, and the second width may be about 220 μm, about 230 μm, about 240 μm, about 245 μm, about 250 μm, about 255 μm, or about 260 μm.

Additionally, or alternatively, the first segment (e.g., first segment 212E) may have a first pitch that is about 0.03 twists/mm to about 0.2 twists/mm larger than a second pitch of the second segment (e.g., second segment 214E), or vice versa. For example, the first pitch may be about 0.65 twists/mm to about 1.5 twists/mm, and the second pitch may be about 0.5 twists/mm to about 1 twist/mm (including all values and sub-ranges therein). In some variations, the first pitch may be about 0.60 twists/mm, about 0.65 twists/mm, about 0.70 twists/mm, about 0.75 twists/mm, about 0.77 twists/mm, about 0.80 twists/mm, about 0.85 twists/mm, or about 0.87 twists/mm, about 0.9 twists/mm, about 0.93 twists/mm, about 1 twist/mm, about 1.1 twists/mm, about 1.17 twists/mm, or about 1.25 twists/mm, and the second pitch may be about 0.4 twists/mm, about 0.45 twists/mm, about 0.50 twists/mm, about 0.55 twists/mm, about 0.57 twists/mm, about 0.60 twists/mm, about 0.65 twists/mm, about 0.67 twists/mm, about 0.7 twists/mm, about 0.75 twists/mm, about 0.8 twists/mm, about 0.84 twists/mm, about 0.9 twists/mm, about 0.95 twists/mm, about 1 twist/mm, or about 1.01 twists/mm. In some variations, the first pitch may be at least 1 times the second pitch, such as about 1.05 times the second pitch, about 1.1 times the second pitch, about 1.15 times the second pitch, about 1.2 times the second pitch, about 1.21 times the second pitch, about 1.22 times the second pitch, about 1.23 times the second pitch, about 1.24 times the second pitch, about 1.25 times the second pitch, about 1.26 times the second pitch, about 1.27 times the second pitch, about 1.28 times the second pitch, about 1.29 times the second pitch, about 1.3 times the second pitch, about 1.35 times the second pitch, about 1.4 times the second pitch, about 1.45 times the second pitch, about 1.5 times the second pitch, about 1.6 times the second pitch, about 1.7 times the second pitch, about 1.75 times the second pitch, about 1.8 times the second pitch, about 1.9 times the second pitch, about 2 times the second pitch, or greater than about 2 times the second pitch. For example, in some variations, the first pitch may be about 1 twist/mm (e.g., about 1.01 twists/mm), and the second pitch may be about 0.8 twists/mm. As another example, in some variations, the first pitch may be about 0.85 twists/mm (e.g., 0.84 twists/mm), and the second pitch may be about 0.65 twists/mm (e.g., 0.67 twists/mm). As yet another example, in some variations, the first pitch may be about 1.15 twists/mm (e.g., 1.17 twists/mm), and the second pitch may be about 0.95 twists/mm (e.g., 0.93 twists/mm).

Additionally, or alternatively, the first segment (e.g., first segment 212F) may have a smaller ROC than the second segment (e.g., second segment 214F), or vice versa. In some variations, a first ROC of the first segment 212F may be about 0.5 mm to about 3 mm less than a second ROC of the second segment 214F. For example, the first ROC may be about 1 mm to about 7 mm, and the second ROC may be about 3 mm to about 10 mm (including all values and sub-ranges therein). In some variations, the first ROC may be about 5.5 mm, about 5 mm, about 4.5 mm, about 4 mm, about 3.5 mm, or about 3 mm, and the second ROC may be about 4 mm, about 4.5 mm, about 5 mm, about 5.5 mm, about 6 mm, about 6.5 mm, or about 7 mm.

In certain instances, it may be beneficial to utilize an implant with a proximal segment of the central portion (and/or a proximal portion of the implant body) that has a larger diameter, a greater twist pitch, and/or a smaller ROC than a distal segment of the central portion (and/or a distal portion of the implant body). For example, one or more of these features may allow the implant to maintain patency of Schlemm's canal by holding open (e.g., tenting) an opening (e.g., at a goniotomy site) through which the implant is inserted into the canal. Thus, aqueous fluid may flow through the opening and into the anterior chamber.

Generally, a distal portion of the implant body extends distally from the central portion of the implant body and constitutes the first portion of the implant to be advanced out of an implant delivery device and into Schlemm's canal during an implantation procedure. That is, once the implant is placed within the canal, the distal portion may be the furthest portion of the implant from the opening created to place the implant (described in detail herein). In some variations, the distal portion (e.g., a portion thereof) may be configured to extend out of (e.g., be exposed from) Schlemm's canal when the implant is positioned within the canal. Generally, the distal portion may be configured to be atraumatic such that it does not pierce, puncture or otherwise damage the trabecular meshwork or other portions of Schlemm's canal during implantation of an implant. As depicted in FIGS. 2A-2B, the distal portion 206A, 206B of the implant may be non-twisted or flat. Compared to the twisted central portion 202A, 202B, the distal portion 206A, 206B may be substantially straight. For example, while the distal end of the distal portion 206 (and thus the implant body) may have a curved configuration so as to be atraumatic, the distal portion 206A, 206B may have substantially planar top and bottom surfaces that are not bent, wound, coiled or otherwise themselves curved into a non-planar configuration. In some variations, the distal portion 206A, 206B may be folded to create a yielding a rounded atraumatic end. For example, a distal end of the portion of the distal portion 206A, 206B (e.g., a fraction thereof, such as ½, ⅓, or ¼ of the distal portion 206A, 206B) may be folded proximally (e.g., toward the proximal portion 204A, 204B) on top of or below a remainder of the distal portion 206A, 206B. In some variations, the distal portion 206A, 206B may be integrally formed with the central portion 202A, 202B of the implant 200A, 200B. Put another way, a distal end of the central portion 202A, 202B and a proximal end of the distal portion 206A, 206B may be of a single construction. In other variations, the distal portion 206A, 206B may be formed separately and coupled to the central portion 202A, 202B using any suitable technique, such as, for example, mechanical fasteners, adhesives, welding or the like. Moreover, as described above, in some variations, the distal portion 206A, 206B may include one or more fenestrations. In one example, the distal portion 206A, 206B may include at least one (e.g., one) fenestration that allows for fluid flow through the distal portion 206A, 206B.

Oppositely, a proximal portion of the implant body generally extends proximally from the central portion of the implant body and constitutes the final portion of the implant to be advanced out of an implant delivery device and into Schlemm's canal during an implantation procedure. That is, once the implant is placed within the canal, the proximal portion may be the closest portion of the implant to the opening created to place the implant. In some variations, the proximal portion (e.g., a portion thereof) may be configured to extend out of (e.g., be exposed from) Schlemm's canal (e.g., through the opening created to place the implant) when the implant is positioned within the canal. The proximal portion may generally be configured to releasably couple to (e.g., engage or interface with) an implant delivery device during implantation. For example, the proximal portion and the implant delivery device (e.g., a distal portion of a positioning tool of the implant delivery device) may form a temporary coupling (e.g., a clearance fit, a transition fit, an interference fit) that is easily decouplable during an implantation procedure (explained in detail herein). As depicted in FIGS. 2A-2B, the proximal portion 204A, 204B may include an engagement mechanism 216A, 216B (e.g., a mating element) for coupling to the implant delivery device, In some variations, the proximal portion 204A, 204B may be integrally formed with the central portion 202A, 202B. Put another way, a distal end of the proximal portion 204A, 204B and a proximal end of the central portion 202A, 202B may be of a single construction. In other variations, the proximal portion 204A, 204B may be formed separately and coupled to the central portion 202A, 202B using any suitable technique, such as, for example, mechanical fasteners, adhesives, welding or the like. Moreover, as described above, in some variations, the proximal portion 204A, 204B may include one or more fenestrations. In one example, the proximal portion 204A, 204B may include at least one (e.g., one) fenestration that allows for fluid flow through the proximal portion 204A, 204B.

The engagement mechanism of the proximal portion (e.g., engagement mechanism 216A, 216B) may be configured to mate with, or otherwise releasably couple to, a corresponding engagement mechanism of the implant delivery device. For example, in some variations, the implant engagement mechanism may be a mating element that is part of a male to female or female to male coupling with a complementary engagement mechanism of the implant delivery device (e.g., on the positioning tool). The implant engagement mechanism may include, for example, one or more of a projection, such as a hook or a tab configured to catch an opening (e.g., a fenestration or an eyelet) of the implant delivery device, and an opening, such as a fenestration configured to receive a projection (e.g., a tab or a hook) of the implant delivery device. In some variations, the engagement mechanism may be integrally formed with a rounded component that is attached to the proximal end of the implant body. For example, the rounded component may be rounded or spherical, and may include one or more engagement mechanisms (e.g., one or more fenestrations therein and/or one or more hooks thereon). Further, the rounded component may be integrally formed with or fixedly attached to (e.g., via fasteners, adhesives, welding, or the like) the proximal end of the implant body so as to engage the delivery device during implantation. In some variations, the body of the rounded component may itself be an engagement mechanism. For example, the delivery device (e.g., a distal portion of a positioning tool thereof) may have a recess configured to receive at least a portion of the rounded component therein. In some variations, fenestrations of the rounded component may additionally or alternatively facilitate coupling of the rounded component to the proximal portion (e.g., when the rounded component is not integrally formed with the implant body). Additionally, the fenestrations may optionally facilitate fluid flow therethrough when the implant is within Schlemm's canal. In some variations, one or more first fenestrations may be configured to aid in coupling the rounded component to the implant body, one or more same or different second fenestrations may be configured to allow fluid flow therethrough, and/or one or more same or different third fenestrations (with respect to one or both of the first and second fenestrations) may be configured to engage with the implant delivery device. In some variations, the rounded component may include a central lumen configured to surround at least part of the proximal portion of the implant body. In some variations, the rounded component may be solid, having neither a central lumen nor fenestrations. In some variations, the rounded component may extend beyond a proximal end of the proximal portion. For example, the rounded component may extend beyond the proximal end by about 0.1 mm to about 5 mm, such as about 0.2 mm to about 4 mm, about 0.3 mm and about 3 mm, about 0.4 mm to about 2 mm, or about 0.5 mm to about 1 mm. In some variations, the rounded component may have a length that is about equal to a thickness of the tissue through which an opening was created to deliver the implant to Schlemm's canal. For example, in certain instances, it may be beneficial to incorporate a rounded component onto the proximal end to form a rounded end that is configured to maintain the patency of the opening created to place the implant within Schlemm's canal and/or facilitate fluid through thorough and/or around the implant when placed within Schlemm's canal.

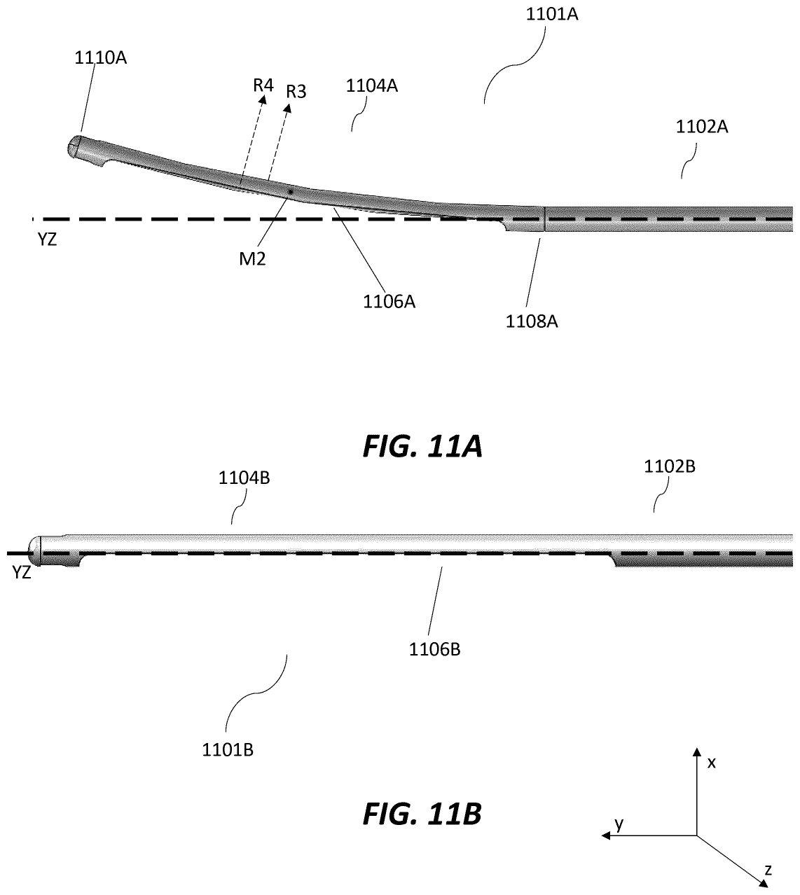

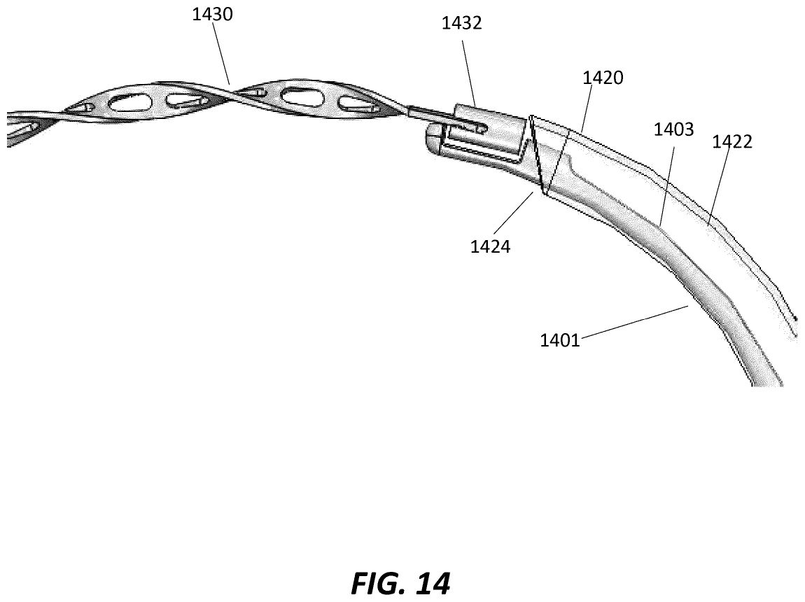

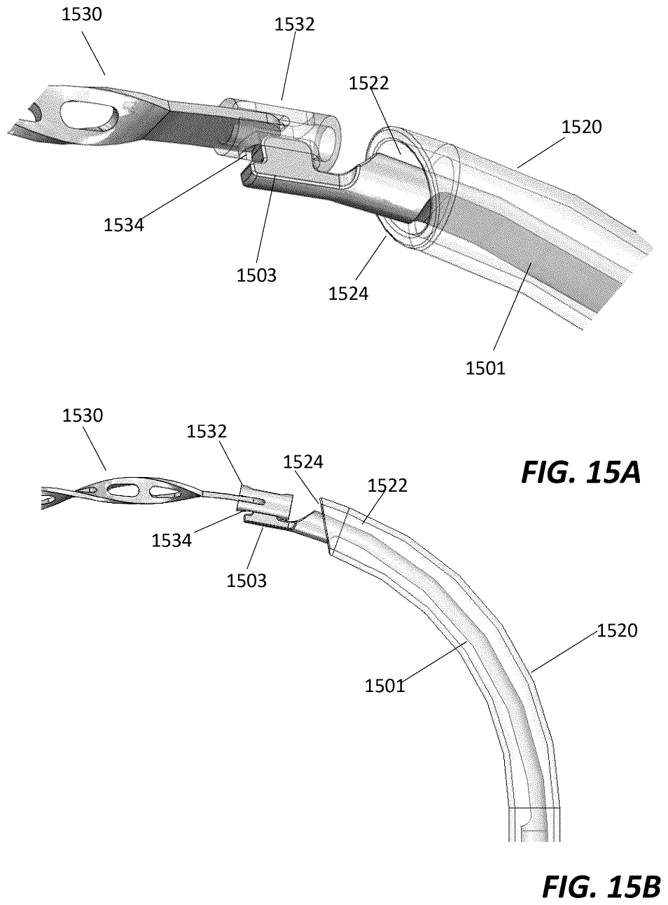

For example, as illustrated in any one of FIG. 2A (and FIGS. 2D-2F), the engagement portion 216A of the proximal portion 204A may include a hook (e.g., a proximal hook) configured to interface with the implant delivery device, such as with an opening and/or a ledge on the implant delivery device (e.g., on a distal portion of a positioning tool of the implant delivery device). The interaction between the proximal hook of the implant and the implant delivery device is described in more detail with respect to FIGS. 11A-11B. Alternatively, as shown in FIG. 2B, the proximal portion 204B may include a cylindrical component, such as a tube coupled thereto and configured to interface with the implant delivery device. As described in more detail with respect to FIGS. 14 and 15A-15B, at least a portion of the tube may be configured to interface with the implant delivery device by, for example, being received within an opening or recess on a distal end of a positioning tool of the delivery device, or by itself receiving a projection (e.g., a tab) on a distal end of the positioning tool.

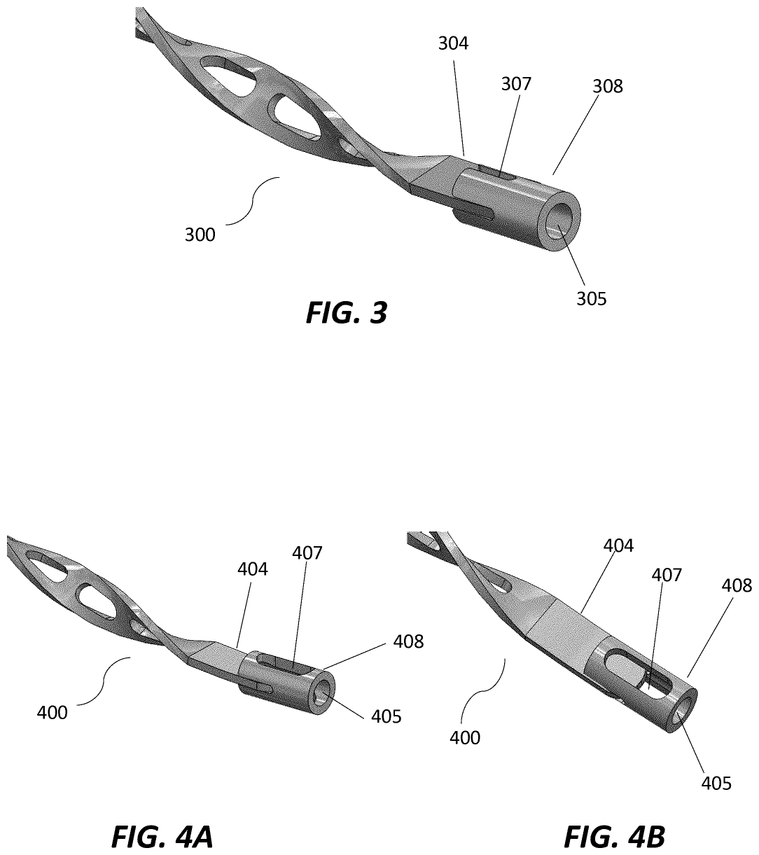

FIGS. 3 and 4A-4B depict variations of a rounded component described above, tube 308, 408, coupled to a proximal portion 304, 404 of an implant 300, 400. The tube 308, 408 may generally include a lumen 305, 405 and/or one or more fenestrations 307, 407 configured to facilitate coupling of the tube 308, 408 to the remainder of the implant body and to optionally facilitate fluid flow therethrough. For example, the tube 308, 408 may include about 1 to about 10 fenestrations 307, 407, such as 2 fenestrations 307, 407 or 4 fenestrations 307, 407. The lumen 305, 405 may be in fluid communication with the fenestrations 307, both of which may facilitate aqueous outflow from Schlemm's canal. As illustrated in FIG. 3, in some variations, the fenestrations 307, 407 may be notches within the tube 308, 408. In particular, the fenestrations 307, 407 may be 3-sided openings originating at a first location of the tube 308, 408 (e.g., proximal to a distal end of the proximal portion 304, 404 or at an intermediate portion of the proximal portion 304, 404) and having a length that is less than a length of the tube 308, 408. In contrast to FIG. 3, which depicts an implant engagement mechanism (in the form of tube 308, 408) comprising fenestrations 307, FIGS. 4A-4B, illustrate a variation of an implant engagement mechanism (in the form of tube 408) comprising fenestrations (e.g., windows) therethrough. In particular, the fenestrations 407 may be 4-sided openings that do not originate at/merge with a perimeter of the tube. For example, the fenestrations 407 may have any of the features (e.g., length, width, shape, etc.) of the fenestrations of the central portion 202A, 202B of the implant described above with respect to FIGS. 2A and 2B.

With respect to fenestrations 307, 407 of all of FIGS. 3 and 4A-4B, a length of the fenestrations may be less than 100% of the length of the tube 308, 408, such as less than about 90% of the length of the tube, less than about 80% of the length of the tube, less than about 70% of the length of the tube, less than about 60% of the length of the tube, less than about 50% of the length of the tube, about 10% to about 90% of the length of the tube, about 20% to about 80% of the length of the tube, about 30% to about 70% of the length of the tube, about 40% to about 60% of the length of the tube, or may be about 50% of the length of the tube. Similarly, a width of the fenestrations 307, 407 may be less than 100% of a width or diameter of the tube 308, 408, such as less than about 90% of the width or diameter of the tube, less than about 80% of the width or diameter of the tube, less than about 70% of the width or diameter of the tube, less than about 60% of the width or diameter of the tube, less than about 50% of the width or diameter of the tube, about 10% to about 90% of the width or diameter of the tube, about 20% to about 80% of the width or diameter of the tube, about 30% to about 70% of the width or diameter of the tube, about 40% to about 60% of the width or diameter of the tube, or may be about 50% of the width or diameter of the tube. Further, one or more fenestrations 307, 407 of a plurality of fenestrations 307, 407 may have one or more unique dimensions.

As is described in detail herein, the tube 308 of FIGS. 4A-4B may be configured to interface with an implant delivery device by receiving one or more portions of the delivery device within one or more fenestrations 407 of the tube 308. For example, a distal end of a positioning tool of the delivery device may include a projection (e.g., a tab or a hook) configured to be received within a fenestration 407 of the tube 308.

In some variations, the proximal portion of the implant may be configured to passively release and/or auto-release from an implant delivery device. For example, the proximal portion may release from the implant delivery device when a user retracts the implant delivery device away from the implantation site (e.g., in a direction away from the small otomy created in the trabecular meshwork for access). In particular, in some variations, just after advancing the proximal portion of the implant out of the implant delivery device and into Schlemm's canal during implantation of the implant—at which time a majority of the implant body is within the canal—the proximal portion may be configured to release from the implant delivery device when a user simply retracts the delivery device away from the implant (e.g., in a direction opposite to the direction of advancement of the implant). In other words, the frictional force between the proximal portion and the implant delivery device (e.g., the distal portion of a positioning tool of the delivery device) may be less than a normal force acting on the majority of the implant (via the trabecular meshwork) such that, when the implant delivery device is moved in a direction different than the direction of the normal force, the trabecular meshwork tissue maintains the entire implant in the direction of the normal force, minimizing or inhibiting its ability to maintain contact with the delivery device. For example, the complementary engagement mechanisms of the implant and the implant delivery device (e.g., a hook and eyelet and/or a tube and recess, etc., as described above) may loosely couple (e.g., via a clearance or transition fit) within the cannula of the delivery device, and the friction of this coupling may be less than the forces of the trabecular meshwork on the remainder of the implant. Accordingly, when a user loosens the coupling between the implant and the implant delivery device by retracting the delivery device, the implant may release from the delivery device and remain within the ocular tissue. This concept is further described herein with respect to the positioning tool of the implant delivery device.

Moreover, in some variations, the implants herein may be configured to be particularly oriented within Schlemm's canal to maintain patency of a portion of the canal. For example, as described above, the implant may have a curvature, this curvature may that correspond to a curvature of Schlemm's canal. That is, the direction of curvature of the implant may be the same as the direction of curvature of the canal. Additionally, the operational alignment of the implant may be defined with respect to a y-axis (e.g., height axis) of the canal, and/or to an iris plane of the eye (a circular plane that bisects a width or diameter of the canal). In particular, one or both of a proximal portion and a distal portion of the implant may be configured to be particularly oriented with respect to the y-axis and/or the iris plane. In other words, the proximal portion may be configured to be oriented in a first alignment (e.g., substantially parallel or substantially perpendicular) relative to the y-axis and/or iris plane, and the distal portion may be configured to be oriented in a second (e.g., substantially perpendicular or substantially parallel) alignment relative to the y-axis and/or the iris plane.

An exemplary operational alignment of a variation of an implant 200 within a cross-sectional, stylized depiction of Schlemm's canal is shown in FIG. 2C. As shown, the implant 200 has a curvature that corresponds to a curvature of Schlemm's canal. The proximal portion 204 of the implant body includes an engagement mechanism (a hook) that is flat or non-twisted with respect to the central portion 202 of the implant body. Similarly, the distal portion 206 of the implant body includes a distal end that is flat or non-twisted with respect to the central portion 202. However, the proximal portion 204 is oriented parallelly to the y-axis and perpendicularly to the iris plane, while the distal portion 206 is oriented perpendicularly to the y-axis and parallelly to the iris plane. For example, the proximal portion 204 may be configured to be placed within about-10 degrees and about 10 degrees of the y-axis of the canal, such as within about-5 degrees and about 5 degrees of the y-axis of the canal, or with about 0 degrees of the y-axis of the canal. The distal portion 206 may be configured to be placed within about-about 80 degrees and about 100 degrees of the y-axis of Schlemm's canal, such as within about 85 degrees and about 95 degrees of the y-axis of the canal, or within about 90 degrees of the y-axis of the canal. As another example, the proximal portion 204 may be configured to be placed within about 80 degrees and about 100 degrees of the iris plane, such as within about 85 degrees and about 95 degrees of the plane, or within about 90 degrees of the plane. The distal portion 206 may be configured to be placed within about-10 degrees and about 10 degrees of the plane, such as within about-5 degrees and about 5 degrees of the plane, or with about 0 degrees of the plane.

Oppositely, in some variations, an implant may be configured to be oriented within Schlemm's canal such that a proximal portion of the implant body is perpendicular to the y-axis of Schlemm's canal and parallel to the iris plane, and a distal portion of the implant body is parallel to the y-axis and perpendicular to the iris plane. For example, the proximal portion may be configured to be placed within about 80 degrees and about 100 degrees of the y-axis of Schlemm's canal, such as within about 85 degrees and about 95 degrees of the y-axis of the canal, or within about 90 degrees of the y-axis of the canal. The distal portion 206 may be configured to be placed within about-10 degrees and about 10 degrees of the y-axis of the canal, such as within about-5 degrees and about 5 degrees of the y-axis of the canal, or with about 0 degrees of the y-axis of the canal. As another example, the proximal portion may be configured to be placed within about-10 degrees and about 10 degrees of the iris plane, such as within about-5 degrees and about 5 degrees of the plane, or with about 0 degrees of the plane. The distal portion 206 may be configured to be placed within about-about 80 degrees and about 100 degrees of the plane, such as within about 85 degrees and about 95 degrees of the plane, or within about 90 degrees of the plane.

Furthermore, exemplary implants that may additionally or alternatively be used with the systems, kits, and/or methods described herein may also be disclosed in U.S. Pat. No. 7,909,789 issued Mar. 22, 2011, and U.S. Pat. No. 8,529,622 issued Sep. 10, 2013 filed Feb. 3, 2011, the contents of each of which were previously incorporated by reference herein.

### Implant Delivery Device

The delivery devices described herein may include implant delivery devices configured to deliver one or more ocular implants to target location within an eye. The delivery devices may be single-handed, single-user controlled devices that may generally include a universal handle having a housing that has a grip portion an interior and a distal end. A cannula may typically be coupled to and extend from the distal end of the housing. The cannula may include a proximal end and a curved distal portion, where the curved distal portion has a proximal end and a distal end, and at least one radius of curvature (ROC) defined therebetween. The cannula may also include a body (e.g., a cylindrical body defining a lumen therein), a distal tip (e.g., a blunt distal tip) defining a distal exit. The distal tip may be integrally formed with the distal end of the curved portion of the cannula (i.e., the tip may directly engage the radius of curvature of the curved distal portion). The delivery devices may also generally include a drive assembly at least partially contained within the housing and having gears that facilitate delivery of an implant. For example, in some variations, the drive assembly may comprise one or more gears that translate rotational movement to linear movement. An implant delivery device may further include a positioning tool having a proximal end and a curved distal portion that is disposed (e.g., movably or slidably disposed) within the cannula lumen. The positioning tool may be configured to advance the implant through the cannula (e.g., via the drive assembly) and decouple from the implant upon retraction of the delivery device from the implantation site and/or automatically upon exposure of at least a part of the distal portion (e.g., a distal end of the curved distal portion) of the positioning tool from the distal tip of the cannula. In some variations, the curved distal portion of the positioning tool may be biased or preset to curve in a direction opposite to a curve of the curved distal portion of the cannula.

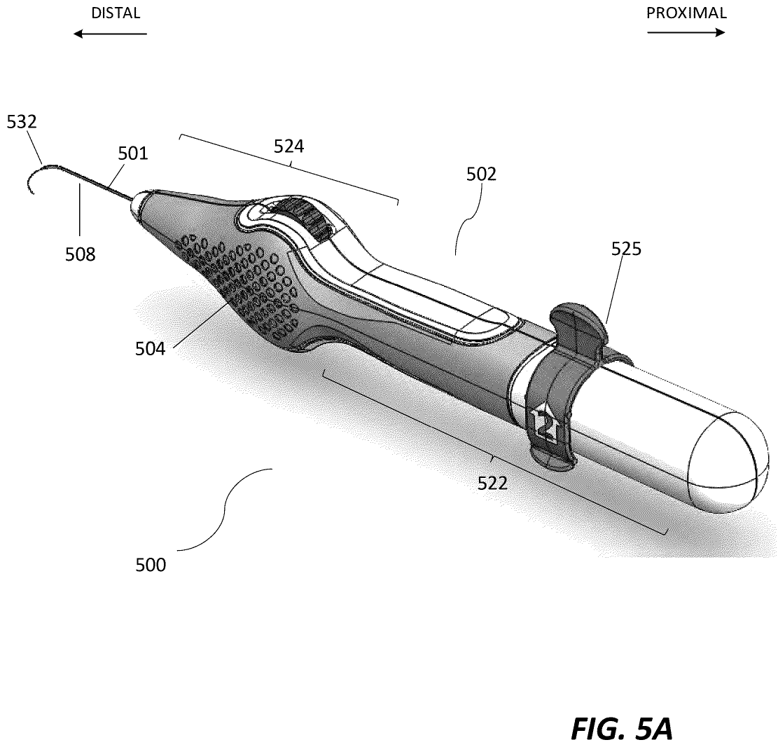

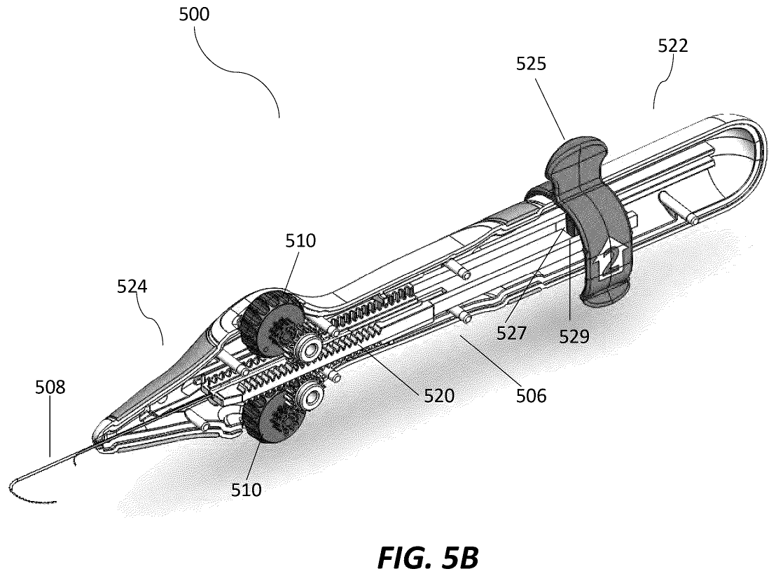

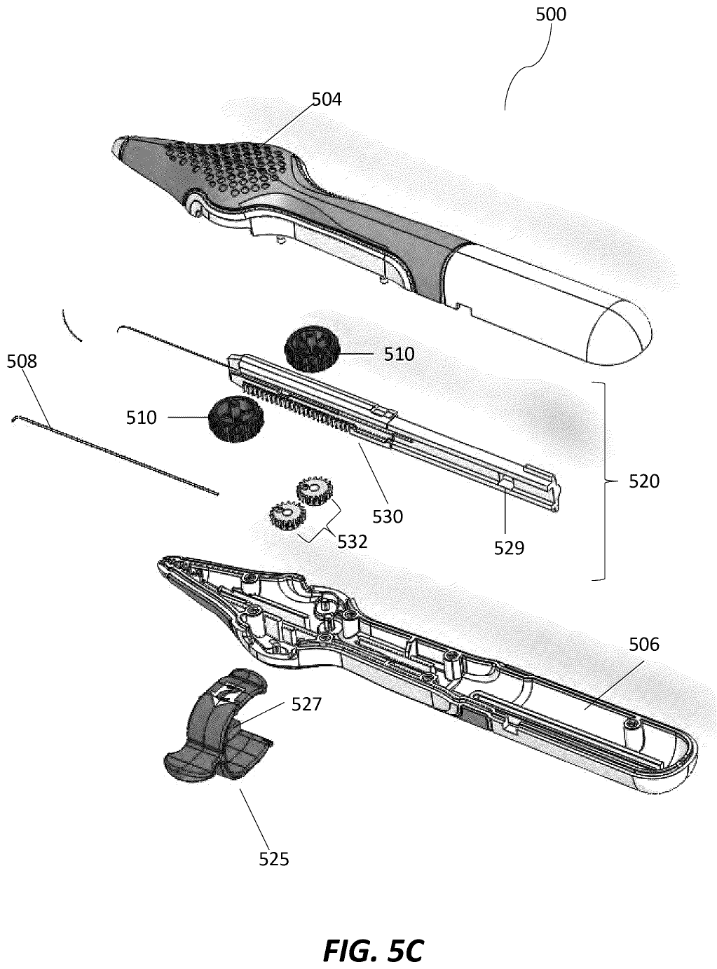

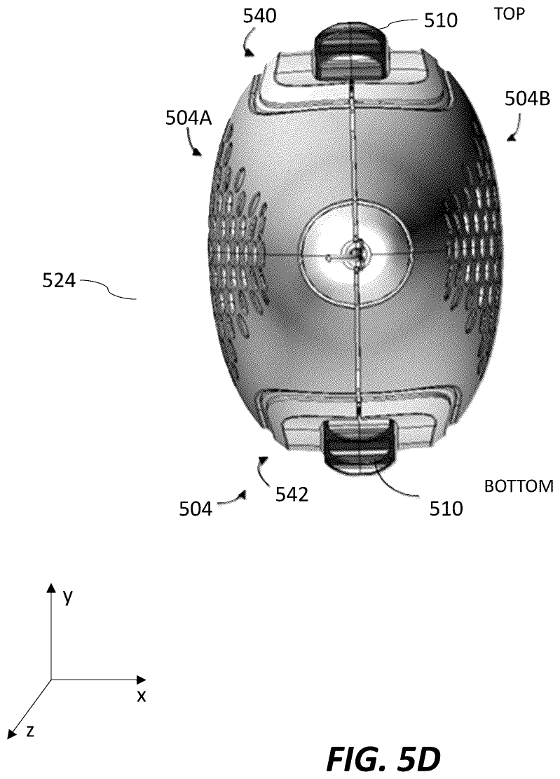

Perspective, cross-sectional, and exploded views of an exemplary implant delivery device 500 are depicted in FIGS. 5A-5C, respectively. As shown, the delivery device 500 may include a handle 502 comprising a housing 506 including a grip portion 504. The housing 506 has a proximal portion and a distal portion. A cannula 508 comprising a distal tip 532 may be coupled to and may extend from the distal portion of the housing 506. The distal tip 532 of the cannula 508 may be configured to provide the user enhanced functionality in seating the cannula 508 in Schlemm's canal, as will be described in more detail herein. The delivery device 500 may further comprise a drive assembly 520 at least partially (e.g., substantially) contained within the housing 506. The drive assembly 520 may be configured to actuate movement of a positioning tool (not shown) within and/or out of the cannula 508. For example, as shown in FIGS. 5B and 5C, the drive assembly 520 may include one or more actuators 510 (e.g., one, two, three, four, or more) such as a rotatable element (e.g., wheel), a slide, a button, or the like, actuation of which (e.g., rotation, translation, depression) may advance and/or retract the positioning tool (not shown) within a lumen of the cannula 508. In some variations, the one or more actuators 510 may extend out of the housing 506 to facilitate user access. For example, the actuators 510 may extend out of the housing 506 of the handle 502, such as, on opposing sides of the handle 502, as depicted in the variation shown in FIGS. 5A and 5B. The actuators 510 may be configured to move (e.g., translate) a linear gear 530 in order to advance and/or retract the positioning tool. As will be described in more detail herein, the handle 502 may be configured, (e.g., the size, shape (including curvatures), placement of portions/components (e.g., actuators, grip portion) of the handle, etc.) to ergonomically fit within the hand of a user to provide easy and comfortable access to the actuators and/or rotation of the handle itself along a longitudinal axis of the handle thus allowing the user to easily control the movement of the positioning tool (not shown).

### Handle

The implant delivery devices described herein may include delivery devices comprising a handle capable of single-handed use by a single user. The handle may be configured such that the ability to use the implant delivery device is independent of which hand a user chooses to use or on which eye a procedure is performed. For example, the handle may be configured for use in the left and right hands and for use on the left and right eyes. The handle may be further configured such that the ability to use the implant delivery device is independent of which direction around Schlemm's canal a tool (e.g., positioning tool, implant) is delivered. For example, the implant delivery device may be used to deliver an implant a clockwise and/or a counterclockwise direction in an eye. However, it should be appreciated that in other variations, the implant delivery devices described herein may be configured to be used in a particular configuration (e.g., with a single side up, only in a clockwise direction, only in a counterclockwise direction, etc.).

Referring back to implant delivery device depicted in FIGS. 5B and 5C, the handle 502 may generally include a housing 506 having a proximal 522 portion and a distal portion 524. The distal portion 524 may generally comprise a grip portion 504 that is configured to be held by a user while actuating the positioning tool, and may contain (e.g., support or carry), or at least partially contain, components of the drive assembly 520 therein. The distal portion 524 may additionally house an internal portion of the cannula 508. The grip portion 504 may be configured to allow the user to grip the handle 502 within about 3 inches, within about 2.5 inches, within about 2 inches, within about 1.5 inches, within about 1 inch, within about 0.75 inches, within about 0.5 inches, or within about 0.25 inches from a proximal end of the cannula 508. In some variations, the grip portion 504 may be configured to allow the user to grip the handle 502 within about 0.25-2 inches from the proximal end of the cannula 508. In some variations, the grip portion 206 may be configured to allow the user to grip the handle 502 within about 0.25-1.5 inches from the proximal end of the cannula 508. It should be appreciated that one or more components of the drive assembly 520 may be configured to translate, and accordingly, may move between the internal cavities of the proximal and distal portions 522, 524 of the housing 502. The proximal and distal portions 522, 524 of the housing may each generally include an interior cavity 523 that may contain (or at least partially contain) internal components of the device, such as components of the drive assembly 520. The distal portion 524 may have the cannula 508 coupled thereto. The grip portion 504 within the distal portion 524 may be raised, depressed, or grooved in certain areas, and/or textured to improve grasp of the handle by the user and/or to improve user comfort, and to ergonomically fit into the hand of a user and control orientation of the handle without requiring wrist rotation.

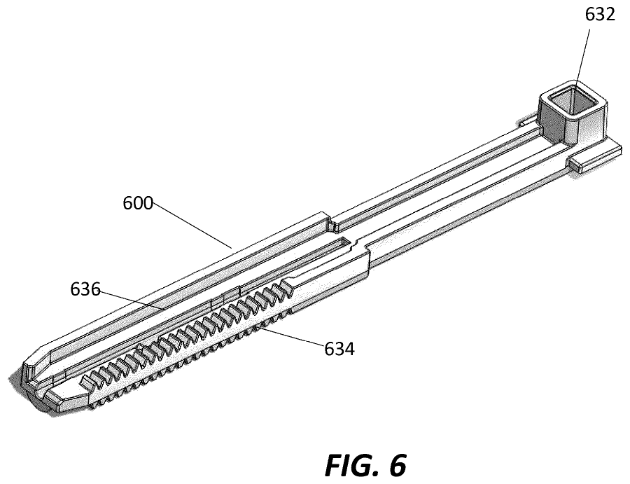

The implant delivery device may be configured to receive a lock therein and/or thereon in order to limit or prohibit actuation of the positioning tool. In some variations, the proximal housing of the handle of the delivery device may receive the lock. For example, a proximal portion of the linear gear (which directly actuates the positioning tool) may have a recess therein configured to receive an extension portion of the lock. The proximal housing may have an opening through which the extension portion is configured to be moved in and out of in order to place with or remove the extension portion (e.g., at least a portion thereof) from the recess of the linear gear. For example, referring again to FIGS. 5A-5C, the proximal portion 522 of the handle housing may be configured to receive a lock 525 at least partially thereon and/or at least partially therein. The lock 525 may comprise a extension portion or projection 527 configured to fit into an opening 529 in the proximal portion 522. In a first configuration, shown in FIGS. 5A and 5B, the extension portion 527 of the lock 525 may be inserted into the opening 529 such it covers the opening 529. Additionally, an exterior portion of the lock 525 may at least partially surround an exterior surface of the proximal portion 522 of the handle housing 506. Further, the extension portion 527 may extend transversely through the proximal portion 522 and may be configured to interface a portion of the drive assembly to maintain a position (e.g., an initial position) of the drive assembly and prevent inadvertent actuation of the drive assembly. For example, the extension portion 527 may be configured to be received within a recess or cavity of a linear gear 530 (e.g., recess 632 of linear gear 630 in FIG. 6) within the interior cavity 523 and stabilize the lock 525 in the first (e.g., locked) configuration. Accordingly, the lock 525 may limit or inhibit a user's ability to actuate the linear gear 530 (and thus the positioning tool (not shown) operably coupled thereto). In a second (e.g., unlocked) configuration, shown in FIGS. 5C, the lock 525 may be removed from the delivery device (e.g., the extension portion 527 may be withdrawn from or otherwise removed from the recess or opening 529), at which point the linear gear 530 may no longer be restricted from moving relative to the housing 506 by the lock 525. A user may remove the lock 525 from the opening 529 using any suitable technique, such as, for example, by rotating, translating, or pulling radially outward the extension portion 527 relative to the handle (and thus, the opening or recess of the linear gear 530).

The handle housing may be fabricated from any suitable material, such as a plastic, a polymer, and/or a metal. In some variations, the handle housing or portions thereof may be made from transparent materials. Materials with suitable transparency are typically polymers such as acrylic copolymers, acrylonitrile butadiene styrene (ABS), polycarbonate (PC), polystyrene, polyvinyl chloride (PVC), polyethylene terephthalate glycol (PETG), and styrene acrylonitrile (SAN). Acrylic copolymers that may be particular useful include, but are not limited to, polymethyl methacrylate (PMMA) copolymer and styrene methyl methacrylate (SMMA) copolymer (e.g., Zylar 631® acrylic copolymer). In variations in which the universal handle is reusable, the handle may be made from a material that can be sterilized (e.g., via autoclaving), such as a heat-resistant metal (e.g., stainless steel, aluminum, titanium) or a high-performance engineering polymer such as PEI, PEEK or PEKK.

The length of the universal handle may generally be about 1 inch to about 20 inches. In some variations, the length of the universal handle may be about 4 inches to 10 inches, such as about 4 inches, about 4.5 inches, about 5 inches, about 5.5 inches, about 6 inches, about 6.5 inches or about 7 inches. In some variations, the length of the universal handle may be about 6 inches to about 7 inches, such as about 6.5 inches, or about 7 inches.

The handle may be configured for ambidextrous use with an ergonomic fit in the hand. To that end, one or more of the proximal and distal portions of the handle may be configured to be symmetrical across one or more planes (e.g., two planes), such as a YZ-plane and a XZ-plane. Such a configuration may make it easier for a user to rotate the handle (e.g., about 10 degrees to about 180 degrees or more) around a longitudinal axis of the handle during use. In some variations, such a configuration may make it easier for the user to rotate the handle about 15 degrees to about 30 degrees including about 20 degrees around the longitudinal axis of the handle during delivery of fluid to each hemisphere of Schlemm's canal. In some variations, all portions of the handle may be symmetrical across one or more planes (e.g., YZ-plane, XZ-plane, and a XY plane). In some variations, one or more of the distal portion and the proximal portion may be configured to be symmetrical across one or more planes and may have a non-circular cross-sectional shape, while in other variations, the one or more portions that may be configured to be symmetrical across one or more planes may have a circular cross-sectional shape. For example, in some variations, one or more portions may have a circular cross-sectional shape (e.g., the proximal portion, the distalmost of the distal portion), and one or more portions may have a non-circular cross-sectional shape (e.g., the grip portion of the distal portion). It should be appreciated that the symmetrical nature of the portions of the housing may refer only to the shape of the profile of that portion (e.g., outer surface) and may or may not include internal surfaces of the housing (i.e., the profile of the portion of the housing may be symmetrical across a plane while the internal surface may include different pins, extensions, or other structures configured to interact with or engage internal components of the device).

In some variations, the handle may be as described in U.S. patent application Ser. No. 18/454,585 filed Aug. 23, 2024 and/or in U.S. application filed on Feb. 27, 2025 titled “OCULAR DELIVERY SYSTEM AND METHODS OF USE” to Needleman et al., having an attorney docket number of SGHT-015/01US 328518-2190, the contents of which was previously incorporated by reference herein.

Each of the proximal portions and the distal portions may have a length along a longitudinal axis, and the total length of the handle may be the sum of the lengths of the proximal and distal portions. In some variations, the proximal portion may have a length equal to the length of the distal portion, while in other variations the length of the proximal portion may be greater than, or less than, the length of the distal portion. In some variations, the proximal portion may have a length within the range of about 2 inches to about 7 inches and the distal portion may have a length within the range of about 2 inches to about 7 inches (including all values and sub-ranges therein). For example, the proximal portion may have a length within the range of about 3 inches to about 6 inches including about 4 inches to about 5 inches. The distal portion may have a length within the range of about 3 inches to about 6 inches including about 4 inches to about 5 inches.

Along the length of the handle, the proximal portion may have a first diameter and the distal portion may have a second diameter, different from the first diameter. In some embodiment, the first diameter and the second diameter may be equal. In some variations, the first diameter and the second diameter may each be constant, while in other variations at least one of the first and second diameters (including both diameters) may vary along the length of the handle. In some instances, a first diameter of the proximal portion may be greater than a second diameter of the distal portion or a second diameter of the distal portion may be greater than a first diameter of the proximal portion.

FIG. 5D depicts a variation of a distal portion 524 of an implant delivery device. In some variations, the grip portion 504 of the distal portion 524 may comprise different cross-sectional shapes along the longitudinal axis of the handle. For example, proximally, the grip portion 504 may comprise a circular cross-sectional shape with a first diameter, while a central portion of the grip portion 504 comprising the top surface 540 and the bottom surface 542 and the actuators 510 may comprise an oblong or oval cross-sectional shape with a major axis and a minor axis, and a distal portion (including the distal end) of the grip portion 504 may comprise a circular cross-sectional shape with a second diameter. The major and minor axes may increase distally from the proximal portion of the grip portion 504 comprising the circular cross-sectional shape to a maximum major axis and a maximum minor axis, which may be aligned with the location of the actuators 232, and then may decrease distally until the distal portion of the grip portion 504 comprising a circular cross-sectional shape with the second diameter. In some variations, the first diameter may be larger than the second diameter and the maximum major axis may be larger than both the first diameter and the second diameter. In some variations, the major axis may be at least about 1.5 times to at least about 3 times the minor axis. In some variations, the major axis may be at least about 1.5 times to at least about 2.5 times the diameter of the proximal portion of the handle. The first diameter of the grip portion 504 may be substantially equal to the diameter of the proximal portion of the handle. In some variations, the diameter of the proximal portion of the handle may be greater than the first diameter of the grip portion. In some variations, the difference in the diameters of the different cross-sectional shapes along the longitudinal axis of the handle may provide added benefits. For example, in some variations, the smaller the diameter of the handle, the more degrees of rotation may occur per movement, especially at the distal end.

As noted above, in some variations, the grip portion may comprise different cross-sectional shapes along the longitudinal axis of the handle. In some variations, one or more of the cross-sectional shapes may include a polygonal shape. For example, proximally, the grip portion 504 may comprise a circular cross-sectional shape with a first diameter while the central portion of the grip portion 504 may comprise a polygonal shape having faceted faces (e.g., two or more faces), and the distal end of the grip portion 504 may comprise a circular cross-sectional shape. The polygonal shape may include a triangular prism, a rectangular prism, a pentagonal prism, a hexagonal prism, a heptagonal prism, an octagonal prism, or the like. In some variations, the faceted faces may collectively comprise a radius of curvature. In some variations, the cross-sectional shape of the housing of the handle at the one or more flat regions may be different from the cross-sectional shape at any of: the distal end of the distal portion, the neck, or the proximal portion.

The shape of the handle, the differences in the diameters of the portions of the handle along the longitudinal axis, and the taper of the grip portion may assist in functionally balancing the intentional movement needed to rotate the distal end of the device with the precise control over subtle movement needed during use.

Moreover, the handles described herein may be configured to promote or otherwise facilitate a forward grip (i.e., more distal on the handle), which may provide the user with added control over the cannula. For example, the grip portion of the handle may include a taper (e.g., an elongated nose), as depicted in FIGS. 5A-5D. With respect to FIG. 5d, the grip portion 504 may comprise a taper from the maximum height to the minimum height of the grip portion 504. The taper in the distal portion may promote a forward grip of the handle by the user (i.e., a grip of the distal portion on the handle). Advantageously, a grip of the user located more distally on the handle places the hand of the user in closer proximity to the eye of the patient, allowing the user more control over the cannula. Furthermore, generally the taper from each of the top surface 540 and the bottom surface 542 to a distal end provides a location (e.g., the grip portion 504) for a user to place an index finger distal the actuators 510. The user may seamlessly move their index finger or thumb between the grip portion 504 and the actuators 510 to access Schlemm's canal (e.g., advance the device to Schlemm's canal, puncture the trabecular meshwork, advance the distal tip of the cannula into Schlemm's canal), actuate the elongate member and deliver the fluid composition. Additionally, the taper from a maximum height to the minimum height of the grip portion 504 may generally provide multiple resting points for fingers of the user, which may additionally assist in reducing user fatigue.

The grip portion may be shaped in a way to ergonomically fit into a hand of the user. To this end, in some variations, the grip portion 504 may be symmetric across a YZ plane, as depicted in FIG. 5D. The YZ plane may divide the grip portion 504 into a first grip portion or half 504A and a second grip portion or half 504B that are symmetric across the YZ plane. The first and second grip portions 504A, 504B may each comprise a rounded profile from a first, top surface 540 to a second, bottom surface 542 opposite the top surface 540. In this manner, the grip portion 504 may comprise a first curved side and a second curved side opposite the first curved side. More specifically, the first grip portion 504A may comprise the first curved side and the second grip portion 504B may comprise the second curved side. In some variations, the first grip portion 504A and the second grip portion 504B may each comprise a continuous curved side (e.g., lacking a planar surface) from the top surface 540 to the bottom surface 542.

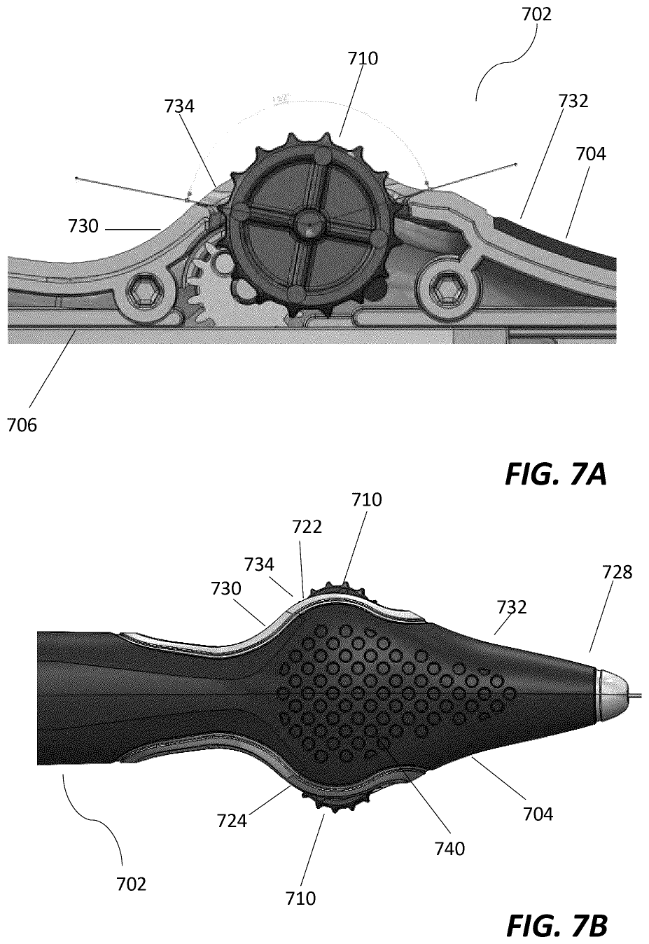

Generally, a grip portion may be symmetric across an XY plane and may comprise a top and a bottom, where each of the top and bottom include an actuator configured to be accessed by a user. Each of the top and bottom may comprise a first curved surface and a straight surface, with a second curved surface therebetween. As seen in FIGS. 7A and 7B, a top of the grip portion 704 may comprise a first curved surface 730 and a straight surface 732, with a second curved surface 734 therebetween. The first curved surface 730 may be proximal of the second curved surface 238 and first curved surface 730 may directly abut the second curved surface 734 (e.g., there may not be any other surface therebetween). Put differently, the distal edge of the first curved surface 234 may be the proximal edge of the second curved surface 734. The first curved surface 730 may be concave while the second curved surface 734 may be convex. An actuator 710 configured to be contacted by a user may be positioned in, through, or otherwise along the second curved surface 734. The straight surface 732 may be distal to both first curved surface 730 and the second curved surface 734. The straight surface 732 may directly abut the second curved surface 734 (e.g., there may not be any other surface therebetween). Put differently, the distal edge of the second curved surface 734 may be the proximal edge of the straight surface 732. The straight surface 732 may be tapered along a longitudinal axis of the handle from a proximal end of the straight surface 732 to a distal end of the straight surface 732. In some variations, the straight surface 732 may terminate at or adjacent to (e.g., just proximal of) the distal end of the handle.

In some variations, the first and second grip portions (e.g., the first and second curved sides) may be symmetric across the cannula. In some variations, the first grip portion and the second grip portion may be symmetric across the XZ plane. In some variations, a portion of each of the first curved side and the second curved side may align with the first and second the actuators, respectively. For example, in some variations, the first and/or second actuator may be longitudinally centered along the first and/or second curved side, respectively.

Turning back to FIG. 5D, in some variations the first grip portion 504A (e.g., first curved side) and the second grip portion 504B (e.g., second curved side) may each be convex or comprise a convex curve. In some variations, the convex curve of the first grip portion 504A and the second grip portion 504B may have a radius of curvature of about 0.3 inches to about 1.5 inches. In some variations, the radius of curvature of the convex curve of the first grip portion 504A and the second grip portion 504B may include ranges of about ⅜ inch to about 1.5 inches, about ½ inch to about 1.5 inches, about ¾ inch to about 1.5 inches, about 1 inch to about 1.5 inches, and about 1.25 inches to about 1.5 inches. In some variations, the radius of curvature of the convex curve of the first grip portion 504A and the second grip portion 504B may include ranges of about 0.3 inch to about 1 inch, about ⅜ inch to about ¾ inch include about ½ inch. The radius of curvature of the convex curve of the first grip portion 504A and the second grip portion 504B may include ranges of about 0.3 inch to about 1.5 inches, about 0.3 to about 1.25 inches, about 0.3 inch to about 1.0 inch, about 0.3 inch to about ¾ inch, about 0.3 inch to about ½ inch, and about 0.3 inch to about ⅜ inch. In some variations, the first grip portion 504A may have a first arc (e.g., a convex arc) with a first center, the second grip portion 504B may have a second arc (e.g., a convex arc) with a second center, and the first and second centers may be on opposing sides of a central longitudinal axis of the cannula and/or a central longitudinal axis of the handle, as seen in FIG. 5D. In some variations, utilizing convex first and second grip portions 504A, 504B may allow the user to re-orient or slightly rotate the grip portion between a thumb and one or more opposing fingers, rather than using the wrist to re-orient or rotate the entire handle. This provides an ergonomic benefit to the user and may help reduce arm fatigue. Furthermore, slight rotational movement of the first grip portion 504A and the second grip portion 504B may translate into movement of the distal tip. For example, in some variations, the first grip portion 504A and the second grip portion 504B may allow a limit of orientation by a user's grip alone to be about +/−30 degrees about a central longitudinal axis, without the user having to rotate at the wrist.

The first and second grip portions 504A, 504B (e.g., first and second curved sides) may each have a one or more radii of curvature between the top and bottom surfaces 540, 542. For example, the first and second grip portions 504A, 504B may each comprise a first, smaller radius of curvature near or adjacent the top surfaces 540, 542 and a second, larger radius of curvature between (e.g., midway) the top and bottom surfaces 540, 542, allowing the grip portion 504 to ergonomically fit into the hand of the user, contacting the hand of the user at or along multiple points to increase control of the device. In some variations, one or more of the first and second grip portions 504A, 504B may each comprise faceted faces (e.g., polygonal comprising two or more faces) from the first, top surface 540 to the second, bottom surface 542 opposite the top surface 540. In some variations, the faceted faces may collectively comprise a curve having a radius of curvature within the ranges of radius of curvature described herein. The faceted faces of one or more of the first and second grip portion 504A, 504B may be configured to control an angle of rotation of the grip portion within the hand of the user. It should be appreciated that while described in the preceding two paragraphs with respect to the first and second grip portions 504A, 504B, such features are also applicable to the first and second curved sides that may form, in some variations, the first and second grip portions respectively.

Moreover, each of the actuators 510 may be positioned on or may otherwise extend from the top and/or bottom surfaces 540, 542 of the grip portion 504. The actuators 510 may extend a defined distance from the top and bottom surfaces 540, 542 and/or may have a distinct shape so that the actuators 510 are easily distinguishable from each of the surfaces 540, 542 themselves when the user is handling the grip portion 504. The top and bottom surfaces 540, 542 may be substantially flat or may otherwise have a large radius of curvature relative to other portions of the handle (e.g., grip portion, neck, proximal portion, thus allowing a user to easily rest a finger on the top and bottom surfaces 540, 542, placing the finger close to but not on the actuators 510.

In some variations, the grip portion be configured to be non-slip and/or at least partially compressible relative to the remainder of the handle. For example, the grip portion may have characteristics that make it easier to hold relative to the remainder of the handle, such as for example, a higher coefficient of friction and a lower durometer. The grip portion may comprise a plurality of materials, including for example, a material with the above-mentioned characteristics overlaid or otherwise covering the material from which the remainder of the housing is formed. For example, in some variations, the housing may be formed of a first material, such as ABS or PC, and the grip portion may further comprise a second material, such as an elastomer (e.g., rubber) coupled to the external surface of the first material. In some variations, a proximal portion of the housing may be formed of a first material, a distal portion of the housing may be formed a second material different from the first material, and the grip portion may be formed of a third material different from the first material and the second material.

An example of a grip portion 704 having such characteristics is depicted in FIG. 7A. There, the grip portion 704 of the handle 702, or a portion thereof, may include a textured surface 740 configured to help the user contact and grip the handle 702. The textured surface 740 may be overlaid on the grip portion 704 and be comprised of a different material than the grip portion 704. The textured surface 740 may include raised elements (e.g., protrusions) and/or indented elements (e.g., impressions, imprints, engraving, etc.), or a combination thereof. For example, raised elements may include bumps and/or indented elements may include circular indentations. Each of the raised or indented elements may be the same size or different sizes (e.g., cross sectional area, diameter, etc.). Each of the raised or indented elements may be the same shape (e.g., triangular, circular, ovular, square, rectangular, hexagonal, octagonal, or the like) or may be different shapes. The textured surface 740 may have a constant pattern across the grip portion 704 or may have a varied pattern configured to enhance tactile feel of the textured surface 740 in hand. For example, in an embodiment, the textured surface 740 may have protrusions of a first height closer to the one or more actuators 710 to help the user rotate the grip portion 704, while the textured surface 740 may have protrusions of a second, different height further away from the one or more actuators 710. In some variations, the first height may be greater than the second height, while in other variations the second height may be greater than the first height. In another embodiment, the textured surface 740 may have indentations of the same shape with a greater cross-sectional area closer to the one or more actuators 710 to increase tactile feel closer to the one or more actuators 710 while the indentations further away from the one or more actuators 710 have a smaller cross-sectional area.

As mentioned above, generally, the handle may be configured such that a user may grasp the handle at the grip portion and still easily access the one or more actuators that may be used to actuate one or more linear gears (e.g., linear gears 530 in FIGS. 5B-5C) to move the positioning tool (now shown) and the implant to Schlemm's canal. As depicted in FIGS. 7A and 7B, a delivery device handle 702 may include one or more actuators, such as, for example, two actuators or at least two actuators 710. The two actuators may include a first actuator on a top side and a second actuator on a bottom (e.g., opposite) side of the handle housing. In some variations, the one or more actuators may include a first actuator on a first side opposite a second side and a second actuator on a third side opposite a fourth side. Each of the actuators 710 may be positioned on or may otherwise extend from top and/or bottom surfaces 722, 724 of the grip portion 704. The actuators 710 may extend a defined distance from the top and bottom surfaces 722, 724 and may have a distinct shape so that the actuators 710 are easily distinguishable from each of the surfaces 722, 724 themselves when the user is handling the grip portion 704. The top and bottom surfaces 722, 724 may be substantially flat or may otherwise have a large radius of curvature relative to other portions of the handle, thus allowing a user to easily rest a finger on the top and bottom surfaces 722, 724, placing the finger close to but not on the actuators 710.

The grip portion 704 may have an hourglass shape, as illustrated in FIG. 7B. The grip portion 704 may taper from the top surface 722 to a distal portion 728 of the grip portion 704. The grip portion 704 may have maximum height at the location along the longitudinal axis having the first and second actuators 710. The grip portion 704 may have a minimum height at the distal portion 728. The tapering of the grip portion 704 from the maximum height to the minimum height may provide the grip portion 704 with a compact feel within the hand of the user, allowing the user to easily rotate the handle 702 around a longitudinal axis to rotate the cannula as needed. Aligning the actuators 710 with the portion of the grip portion that has the maximum height allows the greatest separation between the two actuators 710 allowing the user to clearly distinguish between the two actuators 710 while still being able to control each actuator individually or simultaneously.

When a user's thumb or finger is contacting either actuator 710 or a surface proximal to either actuator 710 (e.g., the top or bottom surface 722, 724) the textured surface 740 of the grip portion 704 may contact multiple locations on the user's hand for stability of the delivery device 500 during delivery of fluid to the eye.

### Drive Assembly

The implant delivery devices described herein may generally include a drive assembly configured to move the positioning tool and deliver an ocular implant into Schlemm's canal. The drive assembly may generally comprise at least a first actuator configured to be contacted by a user and one or more additional actuators configured to move internal components (e.g., the positioning tool) as a result of user actuation of the first actuator. As depicted in FIGS. 5B-5C, the drive assembly 520 or drive mechanism may be at least partially contained within the housing 506 and may include any suitable component or combination of components capable of providing a user with control over the positioning tool (not shown). Exemplary mechanisms by which external input may be converted into motion of one or more components of the implant delivery device 500 are described in more detail herein.

The drive assembly may convert an external input (e.g., motion of a user's thumb or finger) into motion of one or more components of the implant delivery device. More specifically, the drive assembly may cause the positioning tool (not shown) to be advanced through and at least partially distally out of the cannula 508, and/or it may cause the positioning tool (not shown) to be retracted proximally into the cannula 508.

Each of these effects (i.e., extension of the positioning tool (not shown) and retraction of the positioning tool (not shown)) may be actuated using one or more of the same actuators or one or more different actuators. Utilizing the same actuator(s) may allow for easier and more precise single-handed use of the delivery device. For example, if the actuator configured to be contacted by a user comprises a rotatable element (such as the one or more wheels, as in some variations described herein), rotating the rotatable element in a first direction may cause advancement of the positioning tool (not shown), and rotating the rotatable element in a second, opposite direction may cause retraction of the positioning tool (not shown). In variations in which a slide or other first actuator may be used, movement of the first actuator in a first direction (e.g., translational movement of the slide toward a distal end of the implant delivery device) may cause advancement of the positioning tool and movement of the first actuator in a second, different direction (e.g., translation movement of the slide toward a proximal end of the implant delivery device) may cause retraction of the positioning tool. In some variations, the actuator configured to be contacted by a user may have markings or colorings to indicate degree of advancement and/or retraction, and/or direction of advancement and/or retraction.

In some variations, the drive assembly 520 may be configured to allow the implant delivery device to be used only once—that is, the drive assembly 520 may prevent, for example, re-extension of the positioning tool (not shown) after a predetermined amount of extension and/or retraction. In other variations the drive assembly 520 may be configured to allow the positioning tool (not shown) to be advanced, retracted, re-advanced, and re-retracted an unlimited amount.

As mentioned above, the drive assembly 520 may comprise a plurality of actuators, and in some variations, one or more of the plurality of actuators may translate rotational motion into linear motion. For example, as shown in FIG. 5C, the drive assembly 520 may include a linear gear 530 and a pair of pinion gears 532. Each of the pinion gears 532 may also be coupled to an actuator 510 configured to be contacted by a user (e.g., a rotatable wheel). In some variations, such coupling may be accomplished with a pin that can be threaded through a central opening in the actuator 510 and the pinion gear 532, and a nut that secures the rotatable component and pinion gear in a manner so that rotation of the actuator 510 also rotates the pinion gear 532 and vice versa. In some variations, the drive assembly 520 may include a linear gear 530, a pair of pinion gears 532, and at least one actuator 510 coupled to each pinion gear 532. The pinion gears 532 and associated actuator(s) 510 may be disposed on either side of the linear gear 530. In some variations, the pinion gear(s) 532 and the linear gear 530 may contact each other. For example, the teeth of the pinion gear(s) 532 may directly engage corresponding teeth on the linear gear 530, and the actuator(s) 510 on one side of the linear gear 530 may contact the actuator(s) 510 on the opposite side of the linear gear 530. In other variations, the pinion gear(s) 532 and linear gear 530 may be indirectly coupled, for example, via one or more idler gears. The drive assembly 520 may also comprise one or more features to stabilize the pinion gears 532 or otherwise keep them in place. For example, in some variations the drive assembly 520 may comprise actuator (e.g., wheel) spacers configured to sit between axles of the pinion gears 532.

One or more of the actuators configured to be contacted by a user (e.g., actuators 710 of FIGS. 7A and 7B) may have a maximum movement capability (e.g., a maximum rotation for a wheel actuator, a maximum translation for a slide, etc.). For example, in variations in which a rotatable actuator (e.g., rotatable wheel) is utilized, the rotatable actuator may have a maximum rotation of at least 360 degrees, such as about 360 degrees, about 370 degrees, about 380 degrees, about 390 degrees, about 400 degrees, about 420 degrees, about 440 degrees, about 460 degrees, about 480 degrees, about 500 degrees, about 550 degrees, about 600 degrees, about 650 degrees, about 680 degrees, about 690 degrees, about 700 degrees, about 710 degrees, about 720 degrees, about 200 degrees to about 1,000 degrees, about 400 degrees to about 700 degrees, about 300 degrees to about 600 degrees, about 350 degrees to about 550 degrees, about 400 degrees to about 500 degrees, or about 425 degrees to about 575 degrees. The maximum rotation of a rotatable actuator configured to be contacted by a user may correspond to a translation of the positioning tool, relative to a proximal end of cannula (or distal end of the housing), of about 5 mm to about 15 mm, such as about 6 mm to about 14 mm, about 7 mm to about 13 mm, and about 8 mm to about 12 mm (including all values and sub-ranges therein). For example, the maximum rotation of a rotatable actuator may correspond to a translation of the positioning tool of about 9 mm, about 10 mm, about 11 mm, about 11.25 mm, about 11.5 mm, about 11.75 mm, about 12 mm, about 12.25 mm, about 12.5 mm, about 12.7 mm, or about 13 mm. In some variations, actuating a rotatable actuator fewer degrees or millimeters than allowed by its maximum rotation may expose a proximal end of an ocular implant (or at least a portion of the proximal end coupled to a distal end of the positioning tool) from the cannula (e.g., advanced out of a distal tip of the cannula). That is, it may require less rotation than allowed by the maximum rotation to deliver the implant to Schlemm's canal. In some variations, exposing the proximal end of the implant may correspond to advancing the entire length (or substantially the entire length) of the implant out of the cannula. The proximal end of the implant may be exposed due to a rotation of about 20%, about 30%, about 40%, about 50%, about 60%, about 70%, about 80%, about 85%, about 90%, about 95%, less than about 20%, less than about 30%, less than about 40%, less than about 50%, less than about 60%, less than about 70%, less than about 80%, less than about 85%, less than about 90%, or less than about 95%, of the maximum rotation. As an example, given a maximum rotation of about 360 degrees, the proximal end of the implant may be exposed due to a rotation of the rotatable actuator of about 100 degrees, about 125 degrees, about 150 degrees, about 175 degrees, about 180 degrees, about 190 degrees, about 200 degrees, about 210 degrees, about 220 degrees, about 230 degrees, about 240 degrees, about 250 degrees, about 260 degrees, about 270 degrees, about 280 degrees, about 290 degrees, about 300 degrees, about 310 degrees, about 315 degrees, about 320 degrees, about 325 degrees, about 340 degrees, about 350 degrees, about 100 degrees to about 360 degrees, about 150 degrees to about 350 degrees, about 200 degrees to about 340 degrees, about 280 degrees to about 340 degrees, about 300 degrees to about 335 degrees, about 310 degrees to about 330 degrees, or about 315 degrees to about 325 degrees (including all values and sub-ranges therein). Similarly, in some variations, movement of the rotational actuator less than the maximum rotation may cause a distal end of a positioning tool (or at least a portion thereof, coupled to a proximal end of an implant) to be exposed from the cannula (e.g., advanced out of a distal tip of the cannula). In some variations, exposing the distal end of the positioning tool may correspond to advancing about equal to or less than the entire length of an implant out of the cannula. The distal end of the positioning tool may be exposed due to a rotation of about 20%, about 30%, about 40%, about 50%, about 60%, about 70%, about 80%, about 85%, about 90%, about 95%, less than about 20%, less than about 30%, less than about 40%, less than about 50%, less than about 60%, less than about 70%, less than about 80%, less than about 85%, less than about 90%, or less than about 95%, of the maximum rotation. As another example, given a maximum rotation of about 360 degrees, the distal end of the positioning tool may be exposed due to a rotation of the rotatable actuator about 250 degrees, about 260 degrees, about 270 degrees, about 280 degrees, about 290 degrees, about 300 degrees, about 310 degrees, about 315 degrees, about 320 degrees, about 325 degrees, about 340 degrees, about 350 degrees, within about 280 degrees to about 340 degrees, within about 300 degrees to about 335 degrees, within about 310 degrees to about 330 degrees, or within about 315 degrees to about 325 degrees (including all values and sub-ranges therein). Alternatively, in some variations, the one or more actuators configured to be contacted by a user may be configured to be moved (e.g., rotated) indefinitely. That is, the actuator may be rotated and/or otherwise moved (e.g., slid) freely (e.g., may not have a maximum rotation or final position).

Turning to FIG. 6, depicted there is an exemplary linear gear 630 of a drive assembly that is configured to be operably coupled to and configured to be moved as a result of movement of an actuator of the drive assembly. As shown, the linear gear 630 may include at least one side with teeth 634 configured to engage with an actuator of the drive assembly (e.g., with one or more pinion gears), as described above. Additionally, the linear gear may include a central track 636 configured to support an internal portion of the cannula and/or positioning tool of the implant delivery device. For example, an internal portion of the positioning tool (e.g., a portion within the housing of the handle) may be movable (e.g., translatable and/or rotatable) along or within the track 636. Additionally, or alternatively, in some variations, an internal portion of the cannula may be moveable (e.g., translatable and/or rotatable) along or within the track 636.

The linear gear 630 may have a length of about 1 in to about 7 in, such as about 1.5 in to about 6.5 in, about 2 in to about 6 in, about 2.25 in to about 5.5 in, about 2.5 in to about 5 in, about 2.75 in to about 4.5 in, about 3 in to about 4 in, or about 3.25 in to about 3.75 in. in some variations, the length of the linear gear 630 is about 2.7 in, about 2.8 in, about 2.9 in, about 3 in, about 3.1 in, about 3.2 in, about 3.3 in, or about 3.2 in to about 3.3 in. The length of the linear gear may be a fraction of a length of a handle of the delivery device (e.g., handle 506 of FIGS. 5A-5C). For example, the length of the linear gear may be about ⅛^{th}, about 1/7^{th}, about ⅙^{th}, about ⅕^{th}, about ¼^{th}, about ⅓^{rd}, or about ½ the length of the handle. Put another way, the length of the linear gear may be a percentage of the length of handle that is less than 100%, such as about 90%, about 80%, about 70%, about 60%, about 55%, about 50%, about 45%, about 40%, about 30%, about 20%, or about 10% the length of the handle.

Further variations of the drive assembly may not employ translation of rotational motion to linear motion. For example, a slide (e.g., a finger slide) on the handle may be fixed or detachably coupled to a gear within the housing of the handle (e.g., a linear gear as previously described). Here, the drive assembly may be configured so that advancement or retraction of the slide causes advancement or retraction of a positioning tool relative to a cannula of the delivery device.

### Cannula

The cannula of the implant delivery device is typically coupled to and extends from the distal portion of the housing of the handle and is generally configured to provide easy and minimally traumatic, or atraumatic, access to Schlemm's canal, such as during a minimally invasive ab-interno approach. The cannula may generally include a straight proximal portion and a curved distal portion, where the curved distal portion has a proximal end and a distal end (e.g., the distal tip of the cannula), and a radius of curvature (ROC) defined therebetween. The cannula may be configured to guide placement of an ocular implant into at least a portion of Schlemm's canal via the positioning tool (moveable within the cannula). As will be explained in more detail herein, placement of the implant within the canal may be facilitated by variations in which the curved distal portion of the cannula is bent in a first direction and a curved distal portion of the positioning tool therein is preset to bend in a second, different (e.g., opposite) direction. Additionally, the cannula may be used to adjust a position of an ocular implant within the trabecular meshwork after the delivery device has released the implant at least partially within Schlemm's canal. For example, a distal tip of the cannula, or an outer surface of the cannula, may be used to urge a proximal end of the implant (which may still be at least partially within the anterior chamber) toward and into Schlemm's canal, and/or to reorient (e.g., an angle of) or otherwise reposition an implant) relative to (e.g., within) Schlemm's canal.

The cannula may be made from any suitable material with sufficient stiffness to allow it to be advanced through ocular structures (e.g., the cornea, anterior chamber, trabecular meshwork). For example, the cannula may be formed of a metal such as stainless steel, nickel, titanium, aluminum, or alloys thereof (e.g., Nitinol metal alloy) or a polymer. Exemplary polymers include without limitation, polycarbonate, polyetheretherketone (PEEK), polyethylene, polypropylene, polyimide, polyamide, polysulfone, polyether block amide (PEBAX), and fluoropolymers. In some instances, it may be advantageous to coat the cannula with a lubricious coating (e.g., a lubricious polymer) to reduce friction between ocular tissue and the cannula during a procedure. Exemplary lubricious polymers may include, without limitation, polyvinyl alcohol, polyethylene glycol, polyvinyl pyrrolidone, fluorinated polymers (including polytetrafluoroethylene (PTFE or Teflon®)), and polyethylene oxide. In some variations, an interior and/or exterior surface of the cannula may be polished. For example, the interior and/or exterior surface of the cannula may be electropolished with a surface finish of about 0.05 Ra to about 0.9 Ra, such as about 0.075 Ra to about 0.0.85 Ra, about 0.08 Ra to about 0.8 Ra, about 0.09 Ra to about 0.75 Ra, or about 0.1 Ra to about 0.7 Ra (including all values and sub-ranges therein). In some variations, the surface finish may be about 0.1 Ra, about 0.125 Ra, about 0.15 Ra, about 0.175 Ra, about 0.2 Ra, about 0.225 Ra, about 0.25 Ra, about 0.275 Ra, or about 0.3 Ra. An electropolished surface may cause less friction to occur between the surface and an element contacting the surface, such as the positioning tool and/or the implant being delivered by the implant delivery device. Accordingly, electropolishing the interior surface of the cannula may provide for smoother movement of the positioning tool and/or implant within the cannula. In some variations, the surface finish of the interior and/or exterior surface of the cannula may be a mirror polish achieved by any suitable method, such as rough grinding, fine grinding, chemical solution polishing, and/or the like.

The cannula generally has an outer diameter sized to gain access to the lumen of Schlemm's canal while minimally obstructing the surgeon's view. Accordingly, the outer diameter may range from about 50 microns to about 1000 microns. In some variations, the outer diameter may range from about 150 microns to about 800 microns, from about 200 microns to about 700 microns, from about 300 microns to about 600 microns, or from about 400 microns to 500 microns (including all values and sub-ranges therein). The cannula also has an inner diameter, which may range from about 50 microns to about 400 microns, from about 100 microns to about 350 microns, or from about 150 microns to about 300 microns (including all values and sub-ranges therein). The cannula may also be formed to have any suitable cross-sectional profile, e.g., circular, elliptical, triangular, square, rectangular, or the like. In some variations, the cannula may comprise a tapered profile along its length (e.g., the outer diameter of the cannula may decrease from a proximal to a distal end of the cannula).

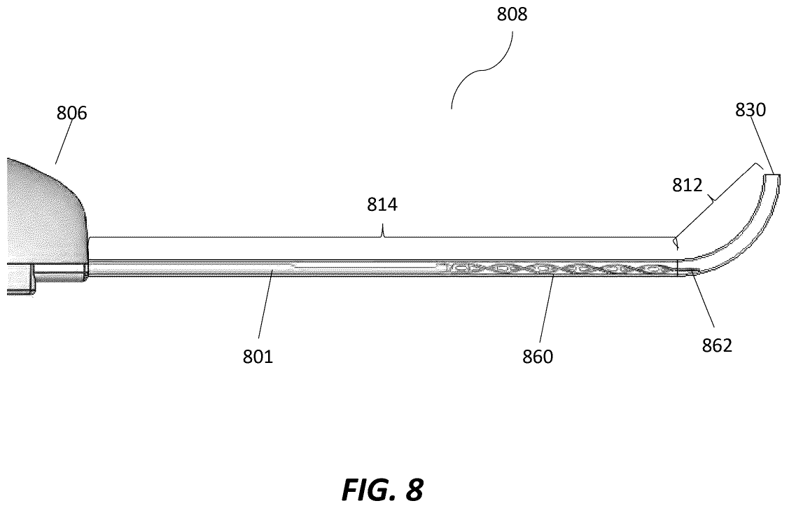

Turning to FIG. 8, an exemplary variation of an implant delivery device 800 shows the geometry of a cannula 808 and how a positioning tool 801 and implant 860 are stored therein. There, a proximal end of cannula 808 is fixedly attached to a distal portion of the handle housing 806. In other variations, the cannula 808 may be rotatably attached to the distal portion of the housing 806 (e.g., via a rotatable hub or the like). In variations of the delivery device where the handle is reusable and the cannula is disposable, the cannula may be removably attached to the distal portion of the housing 806. Within a lumen of the cannula rests a positioning tool 801, which is coupled to an implant 860. In some variations, after an ocular treatment system (e.g., a system including a delivery device and an ocular implant) is assembled (e.g., during storage, prior to use) the implant 860 and the positioning tool 801 may be positioned such that a distal end 862 of the implant resides within a curved distal portion 812 of the cannula. An angular span of the curved distal portion 812 may be defined by an inner radius of the cannula 808, an outer radius of the cannula 808, or central curve having a radius originating from a midpoint between the inner and outer radii of the cannula 808, where the inner radius is about equal to or less than the outer radius. Meanwhile, the positioning tool 801 may rest within a straight proximal portion 814 of the cannula. Together, the straight proximal portion 814 and the curved distal portion 812 may have a length ranging from about 5 mm to about 50 mm, about 10 mm to about 30 mm, or from about 14 mm to about 20 mm (including all values and sub-ranges therein). In some variations, the cannula 808 may have a length of about 18 mm.

The curved distal portion of the cannula may include features to provide minimally traumatic/atraumatic entry into Schlemm's canal. For example, as shown in FIG. 8, the cannula 808 may have a distal tip 830 at a distalmost end of the curved distal portion 812. The distal tip 830 may be integrally formed with the curved distal portion 812. The size, shape, geometry, and the like, of the distal tip 830 may provide minimally traumatic or atraumatic access to Schlemm's canal. In some variations, the distal tip 830 may be blunt. In other words, a face or cross-section of the distal tip 830 may be perpendicular (or substantially perpendicular) to a curve of the curved distal portion 812. Put another way, the inner and outer surfaces of the cannula 808 may merge at a flat edge of the distal tip 830. In this manner, the distal tip may not be pointed, sharpened, or otherwise angled. This bluntness may reduce risk of the cannula unnecessarily disrupting tissue during an implantation procedure.

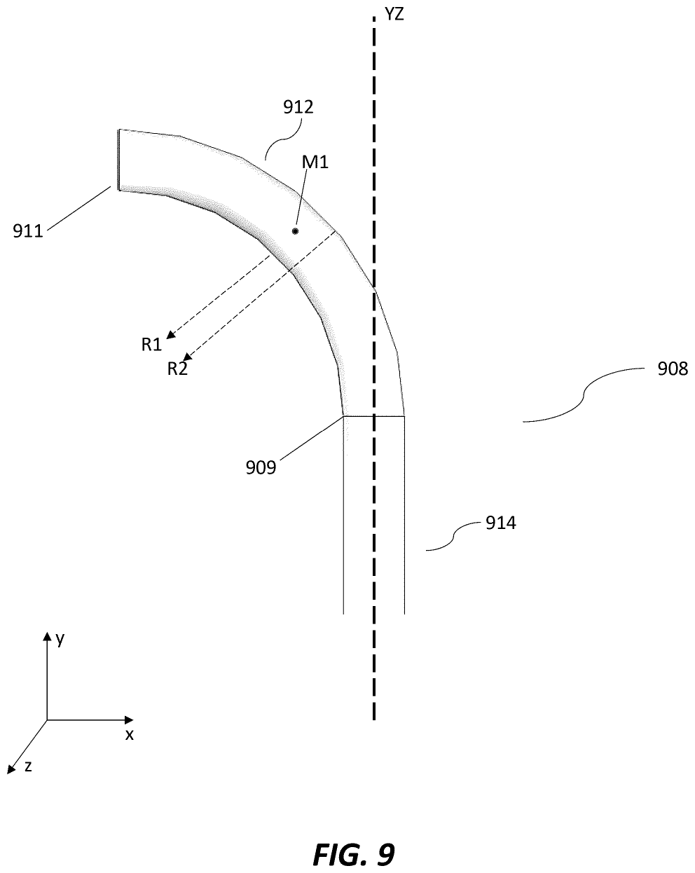

The curved distal portion of the cannula may additionally be shaped to facilitate tangential entry of an implant into Schlemm's canal. Referring to FIG. 9, the curved distal portion 912 of the cannula 908 may have a first radius of curvature (ROC) between proximal and distal ends 909, 911 of the curved distal portion 912 and defined by the inner radius R1 of the curve (e.g., arc, bend). Additionally, the curved distal portion 912 may have a second ROC between proximal and distal ends 909, 911 of the curved distal portion 912 defined by the outer radius R2 of the curve. In some variations, the inner radius R1 may be less than the outer radius R2. In some variations, the first and second ROCs of the curved distal portion 912 may be within about 0.1 mm and 1 mm of each other or may be substantially the same. For example, each of the first and second ROCs may be about 1 mm to about 5 mm, such as about 1.5 mm to about 4 mm, about 2 mm to about 3.5 mm, or about 2.5 mm to about 3 mm (including all ranges and subranges therein). In some variations, each of the first and second ROCs may be within about 1 mm to about 10 mm, or from about 2 mm to about 5 mm (including all values and sub-ranges therein). In one variation, one or both of the first and second ROCs may be about 2.5 mm. In some variations, an ROC of the curved distal portion of the cannula (e.g., or central curve having a radius originating from a midpoint M1 between the first and second ROCs defined by R1 and R2, respectively) may be different than an ROC of the curved distal portion of the positioning tool. For example, the ROC of the curved distal portion of the cannula may be less than an ROC of the curved distal portion of the positioning tool.

The curved distal portion of the cannula may have a curvature (e.g., a preset curvature) that may be defined relative to an axis or plane, such as relative to the y-axis and/or YZ-plane shown in FIG. 9. The y-axis may be defined by a central longitudinal axis of a proximal straight portion 914 of the cannula 908. In some variations, the curved distal portion 912 of the cannula 908 may have a curve that is tangential to this longitudinal axis such that it bends in or toward a first direction relative to the longitudinal axis. For example, if the longitudinal axis is part of a plane, such as an YZ-plane that bisects the straight proximal portion 914 of the cannula 908, the curved distal portion 912 of the cannula may have a curve that is tangential to the y-axis or YZ plane and bends toward the −x direction, as shown. In other words, a curve of the curved distal portion 912 (e.g., defined by the inner radius R1, the outer radius R2, or a central curve therebetween) bends toward a first side of the plane. In some variations, the curve defined by the inner radius R1 of the cannula may be more displaced toward/on the first side of the plane than the curve defined by outer radius R2 of the cannula. It should be understood that the curved distal portion 912 may alternatively bend toward the X direction, the Z direction, the −Z direction, the Y direction, or the −Y direction. That is, it also be understood that the same concept described above applies relative to the XY-plane and the ZX-plane, and depends on a position of the cannula in space.

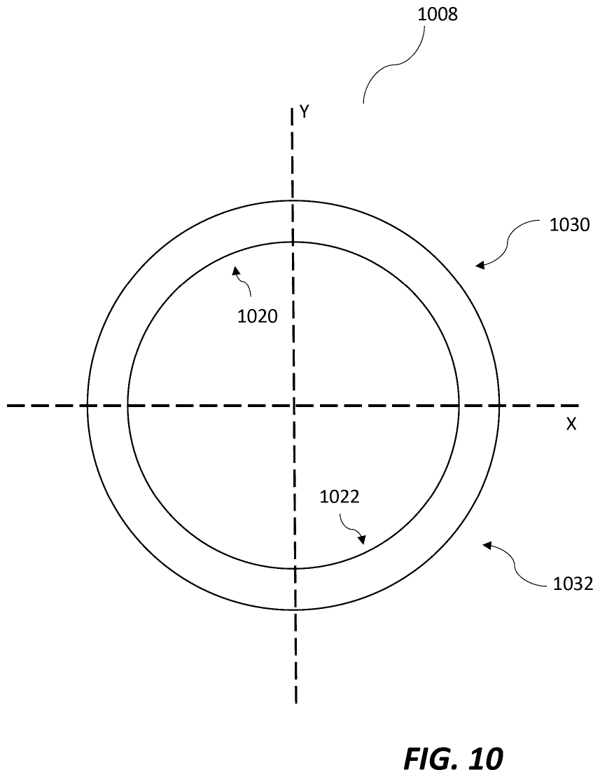

The curved distal portion of the cannula may generally be uniform in cross-sectional shape. For example, FIG. 10 shows a stylized, cross-sectional front view of cannula 1008 with a circular cross-sectional shape. The XY-plane shown bisects the cannula 1008, defining first (top) cross-sectional arcs 1020, 1030 of inner and outer surfaces of the cannula 1008, respectively, and second (bottom) cross-sectional arcs 1022, 1032 of inner and outer surfaces of the cannula 1008, respectively. As will be described in detail herein, the positioning tool (not shown) may be configured to contact and/or cause the implant (not shown) to contact the inner surface (e.g., inner wall) top cross-sectional arc 1020 of the cannula 1008 during at least a portion of a procedure for delivering the implant to Schlemm's canal. Moreover, any portion of the outer surface (e.g., outer wall) top and bottom cross-sectional arcs 1030, 1032 may be used to manipulate a position and/or orientation of the implant after the positioning tool has released the implant at least partially within Schlemm's canal.

### Positioning Tool

The delivery devices may further include a positioning tool disposed within the lumen of the cannula for controlled implantation of an implant within Schlemm's canal. The positioning tool may generally comprise a proximal end, a distal end, and an engagement mechanism at the distal end. The ocular implant may generally be releasably coupled to the positioning tool engagement mechanism. For example, the implant and the positioning tool engagement mechanism may form a clearance fit, a transition fit, an interference fit or the like. The positioning tool may be advanced (e.g., via the delivery assembly) to advance the implant within the cannula and position the implant in Schlemm's canal. Additionally, or alternatively, the positioning tool may used to retract the implant to help with positioning and/or repositioning of the implant, or to recapture the implant (e.g., via the engagement mechanism) to help with positioning and/or repositioning the implant.

Further, the distal portion of the positioning tool may be biased or preset to have a curve, such as in a curved distal portion, that assists the delivery device in retaining the implant within the delivery device during advancement, retaining the orientation of the implant within and relative to the cannula of the delivery device, and generally enables the positioning tool to controllably guide the implant through the cannula lumen. More specifically, the bias or preset curve of at least a portion of the positioning tool may serve to push the implant (e.g., the proximal portion or a segment thereof) against an opposing curved distal portion (e.g., an inner surface or inner wall thereof) of the cannula during at least a portion of an implant delivery procedure, thus assisting in both retaining the coupling between the implant and the positioning tool and maintaining a rotational orientation of the implant. For example, the positioning tool may push the implant against an inner back wall or inner top wall of the cannula (e.g., the inner top cross-sectional arc 1020 of FIG. 10). The engagement mechanism of the positioning tool need not tightly grip (e.g., via a press fit or the like) the proximal portion of the implant to maintain control of the implant within the cannula during a delivery procedure.

For example, the curved distal portion of the positioning tool may be biased toward or against an inner surface of the cannula (e.g., an inner top surface or inner back surface of the cannula) such that the implant thereon (e.g., a proximal portion and/or proximal end of the implant) is urged in a first direction that is toward an inner wall of the cannula, while the opposing curved distal portion of the inner wall of the cannula urges the implant in a second (e.g., opposite) direction, thereby trapping the implant between the engagement mechanism of the curved distal portion of the positioning tool and an inner surface or wall of the cannula. That is, the curved distal portion of the positioning tool may have a direction of curvature that is counter to a direction of curvature of a curved distal portion of the cannula, and the opposing curves may enforce the coupling between the implant and the engagement mechanism of the positioning tool while the implant is advanced through the cannula via the positioning tool. More specifically, the biased curved distal end of the positioning tool (having the engagement portion) may apply a force (e.g., via the implant coupled thereto) against an inner surface or wall of the cannula. A proximal portion of the implant that is in between the engagement mechanism of the positioning tool and the inner surface of the cannula (e.g., the interior top cross-sectional arc 1020 of FIG. 10), may therefore be secured in or on the engagement mechanism by virtue of the opposing forces of the opposing curves of the curved distal portion of the positioning tool and the curved distal portion of the cannula.

In some variations, at least a portion of the implant, such as at least one surface of the proximal portion of the implant, may be in contact with an inner surface of the cannula during at least a portion of the delivery procedure. Additionally, or alternatively, in some variations, at least a portion of the positioning tool, such as at least one surface of the positioning tool engagement mechanism, may be in contact with an inner surface of the cannula during at least a portion of the delivery procedure. These concepts will be explained in more detail below.

In some variations, the positioning tool may initially be positioned (e.g., stored) within the lumen of the straight proximal portion of the cannula. In some variations, the positioning tool may be stored within the cannula such that at least a portion of the positioning tool (e.g., part of a distal portion of the position tool) is within at least a portion of a curved distal portion of the cannula. A length of the positioning tool may be about the same as or greater than a length of a straight proximal portion of the cannula, or vice versa. The length of the positioning tool may be about 1 mm to about 100 mm, such as about 10 mm to about 95 mm, about 20 mm to about 90 mm, about 30 mm to about 85 mm, about 40 mm to about 80 mm, about 50 mm to about 75 mm, or about 60 mm to about 70 mm (including all values and sub-ranges therein). For example, the positioning tool may have a length of about 50 mm, about 52.5 mm, about 55 mm, about 57.5 mm, about 60 mm, about 62.5 mm, about 65 mm, about 67.5 mm, about 70 mm, about 71 mm, about 72 mm, about 73 mm, about 74 mm, about 75 mm, about 76 mm, about 77 mm, about 78 mm, about 79 mm, about 80 mm, about 82.5 mm, about 85 mm, about 87.5 mm, about 90 mm, about 92.5 mm, about 95 mm, about 97.5 mm, or about 100 mm. As another example, the length of the positioning tool may be about 1 mm to about 12 mm, such as about 2 mm to about 11 mm, about 3 mm to about 10 mm, about 4 mm to about 9 mm, about 5 mm to about 8 mm, or about 6 mm to about 7 mm (including all values and sub-ranges therein). In some variations, the length of the positioning tool may be about 6 mm to about 8 mm, such as about 6.25 mm to about 7.75 mm, about 6.5 mm to about 7.5 mm, or about 6.75 mm to about 7.25 mm (including all values and sub-ranges therein). In some variations, the length may be at least 4 mm, at least 5 mm, at least 6 mm, at least 7 mm, at least 8 mm, or at least 9 mm. In some variations, the length of the positioning tool may be about 7 mm.

The positioning tool may have any suitable cross-sectional shape, such as circular, ovular, square, rectangular, triangular, or the like. In some variations, the positioning tool may have an irregular cross-sectional shape. The positioning tool may have a cross-sectional dimension (e.g., a width or diameter) that is about equal to or less than a cross-sectional dimension of the cannula (e.g., inner diameter), such as about 50 microns to about 400 microns. In some variations, the positioning tool may be configured (sized and shaped) to enter Schlemm's canal. For example, the positioning tool may have a cross-sectional dimension (e.g., a cross-sectional width or diameter) of about 50 microns to about 500 microns, such as about 75 μm to about 450 μm, about 100 μm to about 400 μm, about 125 μm to about 375 μm, about 150 μm to about 350 μm, about 150 μm to about 325 μm, about 175 μm to about 300 μm, about 200 μm to about 275 μm, or about 225 μm to about 250 μm. In some variations, the positioning tool may have a cross-sectional dimension (e.g., a cross-sectional width or diameter) of about 25 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm (e.g., 254 μm).

Further, the positioning tool may be formed of any suitable material with the pushability to advance an implant and the flexibility to be advanced through a curved cannula if need be, such as metals and polymers. In some variations, the positioning tool may be formed of a material configured to be shape-set. Exemplary metals for the positioning tool include but are not limited to: stainless steel, nickel, titanium, aluminum, or alloys thereof (e.g., Nitinol metal alloy). Exemplary polymers include but are not limited to: polycarbonate, polyetheretherketone (PEEK), polyethylene, polypropylene, polyimide, polyamide, polysulfone, polyether block amide (PEBAX), and fluoropolymers. In some instances, it may be advantageous to coat the cannula with a lubricious coating (e.g., lubricious polymer) to reduce friction between the positioning tool and the cannula and/or the implant during the procedure. Lubricious polymers include, without limitation, polyvinyl alcohol, polyethylene glycol, polyvinyl pyrrolidone, fluorinated polymers (including polytetrafluoroethylene (PTFE or Teflon®)), and polyethylene oxide. The positioning tool may be cut (e.g., laser cut) from its raw materials. In some variations, at least a portion of the positioning tool (e.g., the entire positioning tool, a distal portion of the positioning tool, a proximal portion of the positioning tool) may be shape set during fabrication, such as by using a heat treatment (e.g., via salt bath, fluidized bed furnace, or the like). The heat treatment may be in the range of about 450 degrees Celsius to about 600 degrees Celsius, such as about 455 degrees to about 590 degrees, about 460 degrees to about 580 degrees, about 465 degrees to about 570 degrees, about 470 degrees to about 560 degrees, about 475 degrees to about 550 degrees, about 480 degrees to about 540 degrees, about 485 degrees to about 530 degrees, about 490 degrees to about 520 degrees, about 495 degrees to about 510 degrees, or about 500 degrees to about 505 degrees. In some variations, all or a portion of the positioning tool may be electropolished (e.g., using one or more cycles) during fabrication. In some variations, the surface finish of the positioning tool may be a mirror polish achieved by any suitable method, such as rough grinding, fine grinding, chemical solution polishing, and/or the like.

As discussed herein throughout, the positioning tool may be shaped to couple with and passively or automatically release an implant during an implant delivery procedure. As shown in FIG. 11A, the curved distal portion 1104A of the positioning tool 1101A may have a first radius of curvature (ROC) between proximal and distal ends 1108A, 1110A of the curved distal portion 1104A and defined by the inner radius R3 of the curve (e.g., arc, bend). Additionally, the curved distal portion 1104A may have a second ROC between proximal and distal ends 1108A, 1110A of the curved distal portion 1104A defined by the outer radius R4 of the curve. In some variations, R3 may be less than R4. In some variations, the first and second ROCs of the curved distal portion 912 may be within about 0.1 mm and 1 mm of each other or may be substantially the same. For example, each of the first and second ROCs may be about 1 mm to about 25 mm, such as about 5 mm to about 20 mm, about 10 mm to about 19 mm, about 11 mm to about 18 mm, about 12 mm to about 17 mm, about 13 mm to about 16 mm, or about 14 mm to about 15 mm (including all values and sub-ranges therein). For example, one or both of the first and second ROCs may be about 5 mm, about 10 mm, about 11 mm, about 12 mm, about 13 mm, about 14 mm, about 15 mm, about 16 mm, about 17 mm, about 18 mm, about 19 mm, or about 20 mm. In some variations, an ROC of the curved distal portion 1104A (e.g., or central curve having a radius originating from a midpoint M2 between the first and second ROCs defined by R3 and R4, respectively) may be different than an ROC of the curved distal portion of the cannula. For example, the ROC of the curved distal portion of the positioning tool may be greater than an ROC of the curved distal portion of the positioning tool.

The curved distal portion of the positioning tool may have a curvature (e.g., a preset curvature) that may be defined relative to an axis or plane, such as relative to the y-axis and/or YZ-plane shown in FIGS. 11A and 11B. The y-axis may be defined by a central longitudinal axis of a proximal straight portion (e.g., proximal straight portion 1102A, 1102B) of the positioning tool. In some variations, the curved distal portion (e.g., curved distal portion 1104A) of the positioning tool may have a curve that is tangential to this longitudinal axis such that it bends in or toward a first direction relative to the longitudinal axis. For example, if the longitudinal axis is part of a plane, such as an YZ-plane that bisects the straight proximal portion 1102A of the positioning tool 1101A, the curved distal portion 1104A may have a curve that is tangential to the y-axis or YZ-plane and bends toward the x direction, as shown. In other words, a curve of the curved distal portion 1104A (e.g., defined by R3, R3, or a central curve therebetween) bends toward a first side of the plane. In some variations, the curve defined by the inner radius R3 of the cannula may be more displaced toward/on the first side of the plane than the curve defined by outer radius R3 of the cannula. It should be understood that the curved distal portion 1104A may alternatively bend toward the −x direction, the z direction, the −z direction, the y direction, or the −y direction. That is, it also be understood that the same concept described above applies relative to the XY-plane and the ZX-plane, and depends on a position of the positioning tool in space.

When the positioning tool includes a curved distal portion, such as curved distal portion 1104A of FIG. 11A, the curved distal portion may have an angular span. For example, an angular span of the curved distal portion 1104A may be defined by R3, R4, or central curve having a radius originating from the midpoint M between R3 and R4. The angular span of the curved distal portion 1104A may be about 1 degree to about 90 degrees, such as about 5 degrees to about 50 degrees, about 10 degrees to about 25 degrees, or about 15 degrees to about 20 degrees.

Alternatively, as shown in FIG. 11B, in some variations, the positioning tool 1101B may be substantially straight along its entire length and/or and may not have a curved distal portion. Put differently, the positioning tool 1101B may have a straight proximal portion 1102B and a straight distal portion 1102B.

Moreover, the positioning tool may have one or more features to enhance the material properties, such as strength and/or flexibility, of the positioning tool. For example, FIGS. 11A and 11B also show a skived portion 1106A, 1106B of the positioning tool 1101A, 1101B. The skived portion may be a recessed or cutaway portion of the positioning tool 1101 that is configured to increase a flexibility of the positioning tool 1101A, 1101B. It may be beneficial to increase the flexibility of the curved distal portion of the positioning tool in order to adjust (e.g., increase) an ROC of the curved distal portion. That is, a more flexible curved distal portion may more easily be biased toward or against an inner surface of the cannula during implantation to secure the implant therebetween. In one example, as is shown in FIGS. 11A and 11B, the skived portion 1106A, 1106B may extend longitudinally along a first side of the positioning tool 1101A, 1101B, where the first side faces in a direction that is opposite the direction of the curve of the curved distal portion 1104A of the positioning tool 1101A. In other variations, the skived portion 1106A, 1106B may extend longitudinally along a second side of the positioning tool 1101A, where the second side faces in the same direction as the direction of the curve of the curved distal portion 1104A.

The skived portion may be fabricated using one or more of laser cutting, plasma cutting, waterjet cutting, flame or oxygen-fuel gas cutting, mechanical cutting, or the like. A length of the skived portion may be about 0.5 mm to about 10 mm, such as about 1 mm to about 8 mm, about 1.5 mm to about 6 mm, or about 2 mm to about 4 mm (including all values and sub-ranges therein). In some variations, the length of the skived portion may be about 3 mm to about 5 mm, such as about 3.25 mm to about 4.75 m, about 3.5 mm to about 4.5 mm, about 3.75 mm to about 4.25 mm (including all values and sub-ranges therein). In some variations, the length may be at least 2 mm, at least 3 mm, at least 4 mm, at least 5 mm, or at least 6 mm. For example, in certain variations, the length of the skived portion may be about 4 mm. In some variations, the length of the skived portion may be about the same as or greater than a length of the curved distal portion. Further, the skived portion may have a depth (e.g., a maximum depth or a minimum depth) of about 10 μm to about 500 μm, such as about 20 μm to about 400 μm, about 30 μm to about 300 μm, about 40 μm to about 200 μm, about 50 μm to about 150 μm, about 75 μm to about 125 μm, about 80 μm to about 120 μm, about 90 μm to about 110 μm, or about 95 μm to about 105 μm (including all values and sub-ranges therein). For example, the depth of the skived portion may be about 75 μm, about 80 μm, about 85 μm, about 90 μm, about 95 μm, about 100 μm, about 105 μm, about 110 μm, about 115 μm, about 120 μm, or about 125 μm. In some variations, the depth of the skived portion may be variable (e.g., angled, such as sloped distally upwards or downwards, or otherwise irregular), while in others, the depth may be constant.

As described in detail herein, the positioning tool may be configured to releasably couple with the implant to facilitate delivery of the implant to a target location within the eye (e.g., Schlemm's canal) and may accordingly include an engagement mechanism configured to interact with and releasably couple to a corresponding engagement mechanism of the implant. For example, in some variations, the implant engagement mechanism may be a mating element that is part of a male to female or female to male coupling with a complementary engagement mechanism of the implant. The positioning tool engagement mechanism may include, for example, one or more of a projection, such as a ledge or a tab configured to catch the implant (e.g., via a hook or a fenestration) of the implant, and an opening, such as a fenestration configured to receive a projection (e.g., hook) of the implant. Generally, the engagement mechanism of the positioning tool may be integrally formed with the tool (e.g., with a body of the positioning tool. For example, the engagement mechanism may be formed using one or more of laser cutting, plasma cutting, waterjet cutting, flame or oxygen-fuel gas cutting, mechanical cutting, or the like. That is, the positioning tool may be cut using a suitable technique in order to create a portion of the positioning tool that is projected from a surface of the positioning tool (e.g., a ledge) or that is an opening extending through the positioning tool (e.g., a fenestration). Further, as is described herein, the curvature of the positioning tool may cause its engagement mechanism to press the corresponding engagement mechanisms of the implant against an inner surface or wall of the cannula during implantation. This action may result in the implant being held between the inner surface of the cannula and the engagement mechanism of the positioning tool, thereby coupling the implant to the positioning tool when the two devices are within the lumen of the cannula.

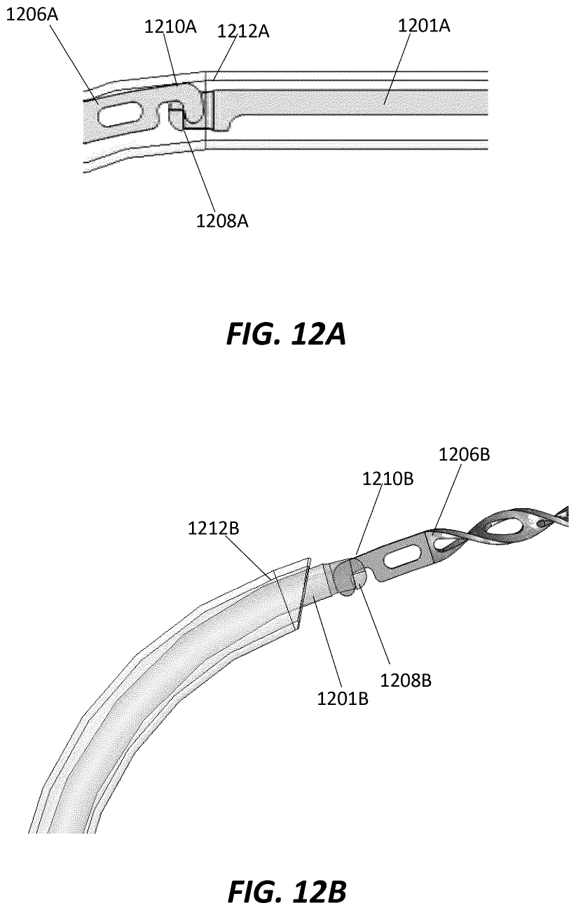

FIGS. 12A and 12B depict exemplary interfaces between a positioning tool engagement mechanism and an implant engagement mechanism. As depicted in FIG. 12A, the positioning tool 1201A and the implant 1204A are coupled within the cannula 1206A. This configuration may exemplify a mid-deployment configuration of the ocular system. The coupling is achieved by the distal ledge 1208A contacting (e.g., catching) the proximal hook 1210A, and by the curvature of the positioning tool 1201A causing the distal ledge 1208A to press the proximal hook 1210A against an inner surface or wall 1212A of the cannula 1206A. This action further causes the proximal hook 1210A to be stuck between the inner surface 1212A and the distal ledge 1208A, thereby effectively coupling the implant 1204A to the positioning tool 1201A when the two devices are within the lumen of the cannula 1206A. Similarly, FIG. 12B shows the distal ledge 1208B engaged with the proximal hook 1210B of the implant 1206B when a distal portion of the positioning tool 1201B is advanced outside of the cannula. In some variations, the positioning tool 1201B may still maintain contact with the inner surface 1212B of the cannula 1206B in this deployment configuration of the system. In some variations, the positioning tool 1201B may not contact the inner surface 1212B of the cannula 1206B in the deployment configuration. That is, in some variations, the positioning tool 1201B may not contact (e.g., push against) the inner surface 1212B during the deployment configuration, when the distal ledge 1208B is advanced out of the cannula 1206B.

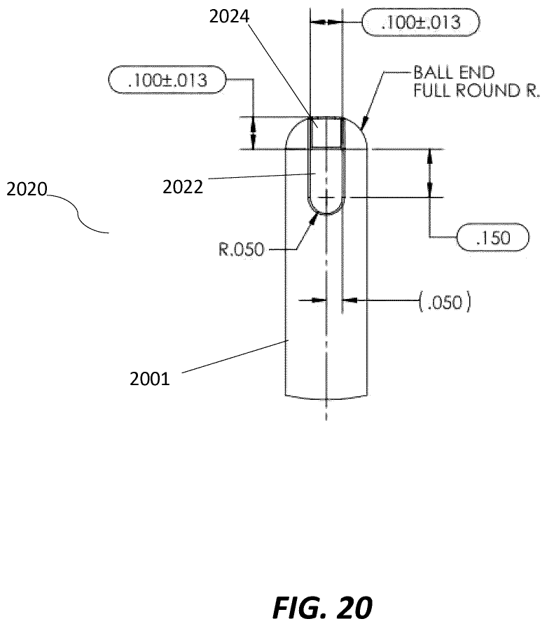

In general, the distal ledge may be a sidewall of an opening or fenestration through the distal portion of the positioning tool. In some variations, the distal ledge may be shorter than the remaining sidewalls of the fenestration. For example, the distal ledge may be skived such that its height is within about 5% and about 95% of the height of the remainder of the fenestration, such as within about 10% and about 90%, within about 20% and about 80%, within about 30% and about 70%, within about 40% and about 60%, within about 45% and about 55%, or about 50% of the height of the remainder of the fenestration. In some variations, the distal ledge may have one or more of the same (or substantially similar) dimensions as a skived portion of the positioning tool (e.g., skived portions 1106A, 1106B of FIGS. 11A to 11B). For example, the skived portion may have a same depth (e.g., a same maximum and/or minimum depth) as the skived portion of the positioning tool. FIG. 20 shows a top view of an exemplary distal portion 2020 of a positioning tool 2001 with fenestration 2022 comprising a distal ledge 2024 (e.g., ledge 1208A, 1208B of FIGS. 12A-12B). A proximal hook of an implant (or other implant engagement mechanism, such as a tab) may catch the fenestration 2022 by extending at least partially through the fenestration 2022. The fenestration may be an opening or window (e.g., may have 4 sides) within the distal portion 2020, and may have cross-sectional dimensions (e.g., a length and width) that are about equal to or less than corresponding cross-sectional dimensions of the positioning tool 2001. The distal ledge 2024 may be a recess or pocket within the distalmost end of the distal portion 2020. The distal ledge 2024 may allow part of the proximal portion of the implant (e.g., a portion of the proximal hook and/or a portion that is distally adjacent to the proximal hook) to rest thereon when a proximal hook (or other implant engagement mechanism, such as a tab) is within the fenestration 2022 (e.g., when the positioning tool and the implant are releasably coupled).

To better understand the coupling between the positioning tool and the implant within the cannula, it may be beneficial to consider the curved distal portion of the positioning tool relative to the curved distal portion of the cannula. For example, as discussed with respect to FIG. 11, a plane bisecting a straight proximal portion of the positioning tool may delineate a first side of the plane and a second, opposite side of the plane. The curved distal portion of the positioning tool may bend toward (e.g., onto) the first side of the plane, while the curved distal portion of the positioning tool may bend away from the first side of the plane (e.g., onto the second side of the plane). That is, if plane is an XY-plane and the straight proximal portion of the positioning tool extends longitudinally in the x direction, the curved distal portion of the positioning tool may curve in the −z direction while the curved distal portion of the cannula curves in the z direction. As another example, if the plane is an XY-plane and the straight proximal portion of the positioning tool extends longitudinally in the −x direction, the curved distal portion of the positioning tool may curve in the z direction while the curved distal portion of the cannula may curve in the −z direction. As yet another example, if plane is an YX-plane and the straight proximal portion of the positioning tool extends longitudinally in the y direction, the curved distal portion of the positioning tool may curve in the z direction while the curved distal portion of the cannula may curve in the −z direction. If the straight proximal portion of the positioning tool extends longitudinally in the −y direction, the curved distal portion of the positioning tool may curve in the −z direction while the curved distal portion of the cannula may curve in the z direction. It should be understood that the same applies relative to the YZ-plane and the XZ-plane. In the same vein, the curved distal portion of the cannula may have a first radius of curvature (ROC) in a first direction, and the curved distal portion of the positioning tool may have a second radius of curvature in a second direction that is different than the first direction. In some variations, the second direction may be opposite to the first direction.

In some variations, when the positioning tool is at least partially within a straight proximal portion of the cannula, a longitudinal axis of the positioning tool defined by its straight proximal portion may be parallel to and/or substantially the same as a longitudinal axis of the cannula defined by the straight proximal portion of the cannula. In some variations, the entire positioning tool, including the curved distal portion, which may be biased to bend counter to a curve of the cannula, may be substantially straight if the entire positioning tool is within the straight proximal portion of the cannula. That is, the straight inner surface or walls of the straight proximal portion of the cannula may maintain a substantially straight configuration of the positioning tool therein. As discussed with respect to FIG. 9, a plane bisecting the straight proximal portion of the cannula may delineate a first side of the plane and a second, opposite side of the plane. The curved distal portion of the cannula may bend toward (e.g., onto) the first side of the plane, while the curved distal portion of the positioning tool may bend away from the first side of the plane (e.g., onto the second side of the plane). That is, if plane is an XY-plane and the straight proximal portion of the cannula extends longitudinally in the x direction, the curved distal portion of the cannula may curve in the z direction while the curved distal portion of the positioning tool curves in the −z direction. Oppositely, if plane is an XY-plane and the straight proximal portion of the cannula extends longitudinally in the −z direction, the curved distal portion of the cannula may curve in the −y direction while the curved distal portion of the positioning tool curves in the z direction. Similarly, if plane is an XY-plane and the straight proximal portion of the cannula extends longitudinally in the y direction, the curved distal portion of the cannula may curve in the −z direction while the curved distal portion of the positioning tool curves in the z direction, while, if the straight proximal portion of the cannula extends longitudinally in the −y direction, the curved distal portion of the cannula may curve in the z direction while the curved distal portion of the positioning tool curves in the −z direction. It should be understood that the same applies relative to the YZ-plane and the XZ-plane.

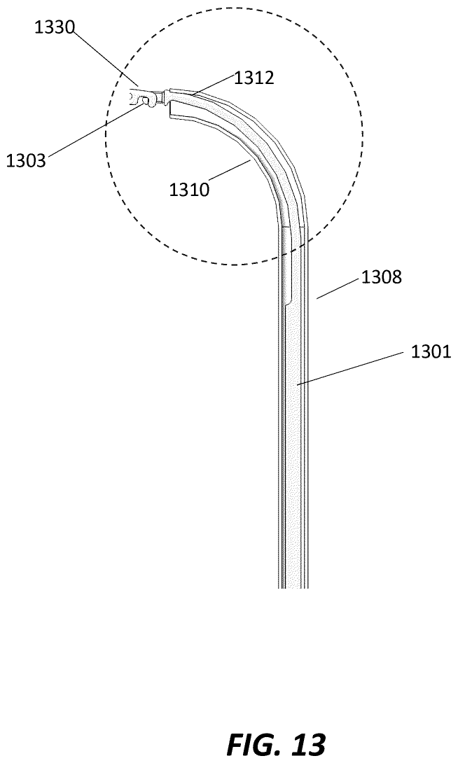

Moreover, as discussed with respect to FIG. 10, a plane bisecting a cross-section of the cannula may form a first side and a second side, where a top cross-sectional arc of the cannula is on the first side and a bottom cross-sectional arc of the cannula is on the second side. Relating this concept to FIGS. 12A and 12B, it should be understood that the curved distal portion of the positioning tool may cause the distal ledge 1208 to urge the proximal hook 1210 against the inner surface 1212 of the top cross-sectional arc of the cannula during advancement of the implant through the cannula (or at least a portion thereof). This feature is depicted in FIG. 13, where a portion of the positioning tool 1301 is within the curved distal portion 1310 of the cannula 1308. As shown, at least a portion of a first side (e.g., backside) of the positioning tool 1301 contacts an inner surface 1312 of the cannula 1308. This contact may occur during at least a portion of a procedure for placing an implant 1330 within Schlemm's canal, such as during a portion of the procedure in which some or all of the implant 1330 has been advanced out of the cannula 1308. for example, as shown, the positioning tool 1301 may contact the inner surface 1312 when the entire implant 1330 has been exposed from the cannula 1308, and/or when at least a portion of the distal end of the positioning tool 1301 (e.g., the distal ledge 1303) has been exposed from the cannula 1308. In some variations, the implant and/or the positioning tool may be in contact with an inner surface of the cannula the entire time that the implant is being advanced through the cannula.

The engagement mechanism (e.g., the distal ledge or distal opening of FIGS. 12A-13) may be configured to passively (or automatically) release the implant therefrom. As described herein throughout, the engagement mechanisms may form a loose coupling (e.g., a clearance fit, or transition fit) between the implant and the positioning tool. The loose coupling may therefore have a low coefficient of friction. Accordingly, when the implant experiences a force that is greater than the frictional force between the engagement mechanisms of the positioning tool and the implant, the implant may react to the larger force and the engagement mechanisms may separate or otherwise become decoupled. As an example, when at least a portion of the implant is within Schlemm's canal, such as when the implant is being advanced out of the delivery device and into Schlemm's canal, the tissue of the trabecular meshwork may exert a force (e.g., an upward normal force) on the implant. When the engagement mechanisms described herein are subsequently exposed from the cannula, the force that was coupling the positioning tool and the cannula (e.g., the downward normal force from the interior of the cannula against the proximal portion of the implant and the upward normal force from the engagement mechanism of the positioning tool against the proximal portion of the implant) is no longer applied. Thus, the force applied by the trabecular meshwork may overcome the smaller friction force between the positioning tool and the implant (due to a loose coupling). In some variations, the implant may automatically release the engagement mechanism when the engagement mechanism is exposed from the cannula (and the force that was coupling the positioning tool and the cannula is no longer applied). In some variations, due to the geometry of the engagement mechanisms and/or the proximal portion of the implant, the implant may passively release from the engagement mechanism of the positioning tool after the engagement mechanism is exposed from the cannula, when the delivery device (including the positioning tool) is move (e.g., retracted) away from the implantation site (e.g., away from the implant, away from the trabecular meshwork and Schlemm's canal, and/or out of the anterior chamber).

Another variation of the engagement mechanism of the positioning tool may comprise a recess or pocket configured to interface with a complimentary engagement mechanism (e.g., a projection) on the ocular implant. Referring to FIG. 14, the positioning tool 1401 may comprise a recess 1403 in a distal portion there of (e.g., distal to a skived portion) configured to receive a corresponding engagement mechanism of the implant. For example, the implant may comprise an engagement mechanism, such as a rounded component, comprising a shape and size that allows the rounded component to be received within the recess 1403 of the positioning tool 1401. In some variations, the rounded component may be a proximal tube 1432 of the implant 1430. The recess 1403 may be shaped to support the proximal tube 1432. Throughout a procedure for delivering the implant 1430 within the eye, the positioning tool 1401 may be configured (e.g., via a preset distal curve of the implant) to push the proximal tube 1432 against the inner surface or wall 1422 of the cannula 1420, thereby holding the proximal tube 1432 within the recess 1403 to maintain the coupling between the positioning tool 1401 and the implant 1430. The positioning tool 1401 may also be configured (e.g., via a preset distal curve of the implant) to passively decouple from the implant 1430 during the delivery. For example, the positioning tool 1401 may decouple from the implant 1430 when the positioning tool pushes the entire proximal tube 1432 out of the distal exit 1424 of the cannula 1420 and/or when the positioning tool 1401 is moved away from the proximal tube 1432.

Yet another variation of the engagement mechanism of the positioning tool may comprise a projection configured to interface with a corresponding opening of the implant. Referring to FIGS. 15A and 15B, the projection of the positioning tool may be in the form of a distal tab 1503, and the opening may be in the form of a fenestration 1534 within a proximal portion (e.g., a proximal tube 1532) of the implant 1530. The distal tab 1503 may be sized and shaped to fit within the fenestration 1534. Throughout a procedure for delivering the implant 1530 within the eye, the positioning tool 1501 may be configured (e.g., via a preset distal curve of the implant) to push, through the fenestration 1534, the proximal tube 1532 against inner surface or wall 1522 of the cannula 1520, thereby holding the distal tab 1503 within the fenestration 1534 to maintain the coupling between the positioning tool 1501 and the implant 1530. The positioning tool 1501 may also be configured (e.g., via a preset distal curve of the implant) to passively detach from the implant 1530 during the delivery. For example, the positioning tool 1501 may detach from the implant 1530 when the positioning tool pushes the entire proximal tube 1532 out of the distal exit 1524 of the cannula 1520 and/or when the positioning tool is moved away from the implant.

### Fluid Delivery Device

Additionally, or alternatively, the delivery devices described herein may include fluid delivery devices configured to a fluid composition to the eye. Like the implant delivery devices described above, the fluid delivery devices may be single-handed, single-user controlled devices that generally include a universal handle having a grip portion and a housing that has an interior and a distal end. A cannula is typically coupled to and extends from the housing distal end. The cannula may include a proximal end and a curved distal portion, where the curved distal portion has a proximal end and a distal end, and a radius of curvature defined therebetween. The cannula may also include a body, a distal tip defining a distal exit, and a lumen extending from the proximal end to the distal tip. The distal tip may be integrally formed with the distal end of the curved portion of the cannula (i.e., the tip may directly engage the radius of curvature). The delivery devices may also generally include a drive assembly at least partially contained within the housing and having gears that translate rotational movement to linear movement. Unlike the implant delivery devices, a fluid delivery device may further include a slidable elongate member disposed within the cannula lumen. In some variations, the slidable elongate member may comprise a lumen therethrough. The delivery devices may also include a fluid assembly housed at least partially in the handle. Fluid compositions such as saline, viscoelastic fluids, including viscoelastic solutions, air, and gas may be delivered using the fluid delivery device. Suitable markings, colorings, or indicators may be included on any portion of the system to help identify the location or position of the distal end of the cannula and/or the slidable elongate member. Exemplary fluid delivery devices that may be used with the devices, systems, kits, and methods described herein are disclosed in U.S. patent application Ser. No. 18/454,585 filed Aug. 23, 2024, and U.S. application filed on Feb. 27, 2025 titled “OCULAR DELIVERY SYSTEM AND METHODS OF USE” to Needleman et al., having an attorney docket number of SGHT-015/01US 328518-2190, the contents of each of which were previously incorporated by reference herein.

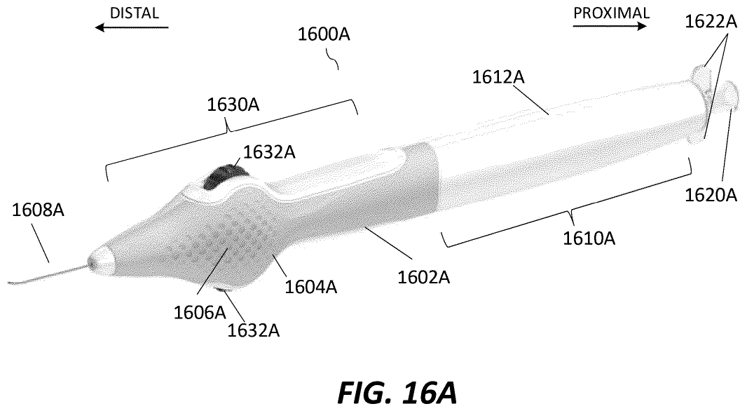

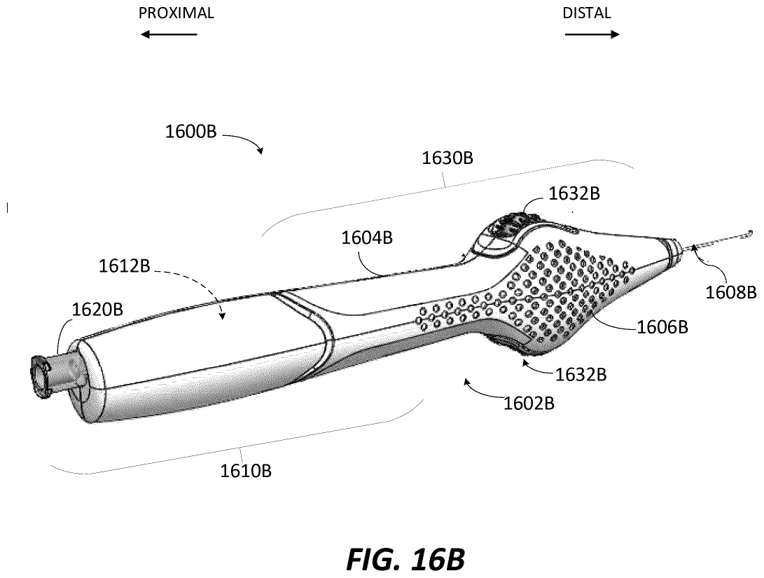

A fluid delivery device may include one or more features of the implant delivery devices described above. For example, FIG. 16A and FIG. 16B depict a front perspective view and a back perspective view, respectively, of variations of a fluid delivery device 1600A/1600B, which may include one or more features that are the same as or substantially similar to those of the implant delivery device 500 of FIGS. 5A-5C. As shown, the fluid delivery devices 1600A/1600B may comprise a handle 1602A/1602B having a housing 1604A/1604B defining an interior volume. The housing 1604A/1604B may comprise a fluid assembly 1610A/1610B at least partially contained in the housing (e.g., in the interior volume thereof), and a cannula 1608A/1608B at its distal end. In some variations, the fluid assembly 1610A/1610B may include a fluid reservoir 1612A/1612B. The fluid reservoir 1612A/1612B may comprise a fluid reservoir connector 1620A/1620B configured to releasably couple with an external fluid device configured to deliver a fluid composition to the fluid assembly 1610A/1610B. In other variations, the fluid reservoir 1612A/1612B may not include a fluid connector and/or may not be configured to receive a fluid in-situ within the handle 1602A/1602B (e.g., the fluid reservoir 1612A/1612B may be configured to be preloaded with a fluid composition or otherwise configured to receive a fluid composition before integration into the handle 1602A/1602B). In some variations, the fluid reservoir connector 1620A/1620B may couple to a fluid reservoir 1612A/1612B of the distal portion of the housing 1604A/1604B (e.g., a distal end) and may be configured to at least partially (e.g., entirely) fill the fluid reservoir 1612A/1612B of the fluid assembly 1610A/1610B with a fluid composition. In some variations, the fluid reservoir connector 1620A/1620B may additionally be configured to provide a fluid composition for irrigation of the operative field and/or purge air from the system. The fluid reservoir connector 1620A/1620B may be configured to receive an external fluid device (e.g., a syringe or fluid coupling in fluid communication with an external fluid reservoir) to transfer fluid into a fluid reservoir of the fluid assembly. In some variations, such as that depicted in FIG. 16A, the fluid reservoir connector 1620A may be configured to be rotated to be released from the handle 1602A (e.g., with the external fluid device coupled thereto). In some variations, the fluid reservoir connector may comprise one or more projections that enable removable attachment of the connector to the handle, such as connector 1620A of FIG. 16A, which comprises tabs 1622A. For example, one or both of the tabs 1622A may be configured to be gripped by an operator to removably couple/decouple the connector 1620A to/from the handle 1602A. In other variations, a fluid reservoir connector may not comprise such projections and/or may not be releasably coupled to the handle, such as connector 1620B depicted in FIG. 16B.

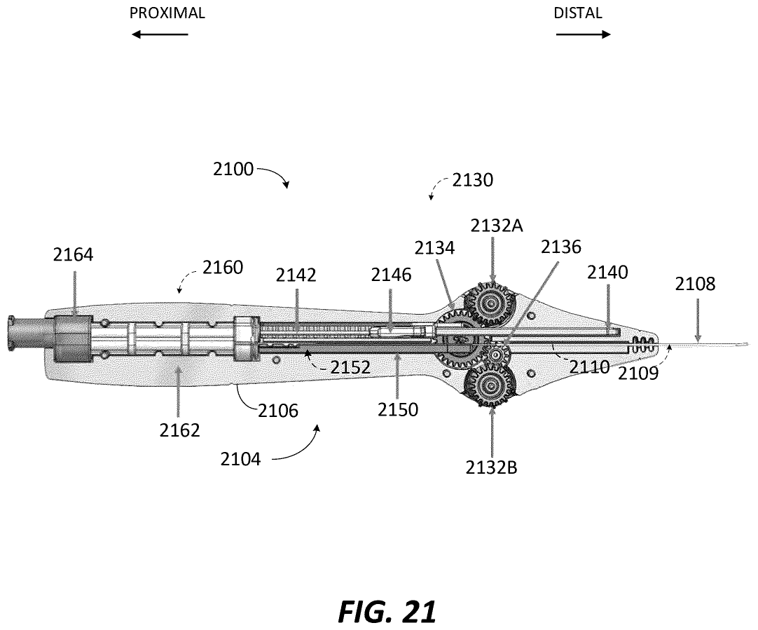

The cannula 1608A/1608B of the fluid delivery device 1600A/1600B may differ from the cannula 508 described with respect to FIGS. 5A-5C. For example, the cannula 1608A/1608B of the fluid delivery device 1600A/1600B may have proximal portion, a straight portion, and a central portion having a distal tip and two or more different radii of curvature (e.g., defined by at least an inner radius and an outer radius of the cannula). Additionally, or alternatively, the distal tip of the cannula 1608A/1608B may be placed within Schlemm's canal and may be used to help form an access point within the trabecular meshwork for entry of the elongate member (which may be configured to disrupt the trabecular meshwork or other similar tissues) into Schlemm's canal. When deployed to Schlemm's canal, the distal tip may help form the access point within the trabecular meshwork. That is, the distal tip may have a cutting edge, such as a bevel, for creating an opening within the trabecular meshwork. The configuration of the distal tip may allow a user to form and use the same access point when delivering fluid to both hemispheres of Schlemm's canal, and in variations of the procedures described herein in which both the fluid delivery devices and implant delivery devices are utilized, the distal tip of the cannula 1608 may of the fluid delivery device 1600A/1600B may allow a user to use the same access point when delivering an implant. Further, the cannula 1608A/1608B of the fluid delivery device may house an elongate member (described below with reference to FIG. 21) instead of a positioning tool. The elongate member may be disposed in the lumen of the cannula 1608A/1608B. In some variations, the elongate member may be solid without a lumen. In other variations, the elongate member may comprise a conduit defining a lumen and be configured to deliver one or more fluid compositions. To deliver the fluid, the elongate member may be fluidly coupled to the fluid reservoir 1612A/1612B for delivering fluid to Schlemm's canal.

Moreover, the fluid assembly 1610A/1610B may include one or more moveable components that together with a drive assembly 1630A/1630B may deliver a fluid composition from the device to the eye. The drive assembly 1630A/1630B may be at least partially contained within the housing 1604A/1604B of the handle 1602A/1602B. In some variations, the drive assembly 1630A/1630B may comprise one or more moveable components including one or more actuators 1632A/1632B configured to be actuated (e.g., rotated) by the hand of a user to deliver a fluid composition (e.g., while simultaneously advancing or retracting the elongate member within Schlemm's canal or the cannula 1608A/1608B or separately from advancing and/or retracting the elongate member). In some variations, the one or more actuators 1632A/1632B may include a single actuator. Alternatively, the one or more actuators 1632A/1632B may comprise a plurality of actuators (e.g., at least one, two, three, four, five, or more than five actuators). In some variations, one or more actuators may be operatively coupled to one or more motors, such that actuation of the one or more actuators of the delivery device may actuate one or more motors to advance and/or retract the elongate member and/or deliver a fluid composition in a motorized fashion. Moreover, the drive assembly 1630A/1630B may optionally include a cannula actuator (not shown) configured for a function different from the one or more actuators 1632A/1632B. For example, while the actuator(s) 1632A/1632B may be configured to translate an elongate member (described in detail below) longitudinally within the cannula 1608A/1608B and/or deliver a fluid composition, the drive assembly 1630A/1630B may include the cannula actuator to rotate the cannula 1608A/1608B about the longitudinal axis of the delivery device 1600A/1600B. The drive assembly is described in further detail with respect to FIG. 21.

FIG. 21 depicts a cross-sectional view of an exemplary fluid delivery device 2100, which may have one or more of the same components the fluid delivery devices 1600A/1600B shown in FIGS. 16A-16B. The delivery device 2100 may comprise a handle 2102 having a housing 2104. A cannula 2108 may be coupled to and may extend from the distal end of the housing 2104 of the handle 2102. The fluid delivery device 2100 may further include an elongate member 2110 slidably positioned within the cannula 2108. The fluid delivery device 2100 may further comprise a drive assembly 2130 at least partially contained within the housing 2104 of the handle 2102. The drive assembly 2130 may comprise one or more actuators 2132 configured to be contacted by a user and one or more additional actuators 2134, 2136, such as linear gears, configured to translate motion from the one or more actuators 2132 configured to be contacted by a user into motion of internal components of the delivery device to ultimately move the elongate member 2110 and/or deliver a fluid composition from the fluid reservoir 2162.

In some variations, the one or more linear gears 2140, 2150 may be configured to translate rotational movement of the one or more actuators 2132 into linear movement, which may be used to move one or more internal components of the delivery device. For example, as shown in FIG. 21, the drive assembly 2130 may comprise a first linear gear 2140 and a second linear gear 2150. In some variations, the first linear gear 2140 and the second linear gear 2150 may be configured to move in opposing directions to facilitate movement of the elongate member 2110 and delivery of a fluid composition during the movement of the elongate member 2110. In some variations, the first linear gear 2140 and the second linear gear 2150 may be configured to move the same distance in opposing directions that may result in advancement of the elongate member 2110 and delivery of a fluid composition from the fluid reservoir 2162 or retraction of the elongate member 2110 and delivery of a fluid composition from the fluid reservoir 2162. Conversely, in some variations, the first and second linear gears 2140, 2150 may be configured to move in the same direction to facilitate movement of the elongate member 2110 and delivery of a fluid composition during the movement of the elongate member 2110.

In some variations, the first and second linear gears 2140, 2150 may be configured to move simultaneously. For example, in some variations, the first linear gear 2140 may be configured to move in a first direction and the second linear gear 2150 may be configured to move simultaneously in a second direction (which may or may not be opposite the first direction) upon actuation of the one or more actuators 2132 configured to be contacted by the user. Such actuation of the one or more linear gears 2140, 2150 may move the elongate member 2110 and/or deliver a fluid composition from the fluid reservoir 2162. In particular, movement of the first and/or second linear gears 2140, 2150 may advance and/or retract the elongate member 2110. Additionally, or alternatively, movement of the first and/or second linear gears 2140, 2150 may deliver a fluid composition from the fluid reservoir 2162 to the eye.

For example, movement of the first linear gear 2140 may move a displacement rod 2142 into the fluid reservoir 2162 to deliver fluid, and movement of the second linear gear 2150 may move a plunger tube 2152 into the fluid reservoir 2162 to deliver fluid. In some variations, both the first and the second linear gears 2140, 2150 may move during one or more of: advancement of the elongate member 2110, retraction of the elongate member 2110, re-advancement of the elongate member 2110, re-retraction of the elongate member 2110, and delivery of a fluid composition from the fluid reservoir 2162 to the eye. Put differently, in some variations, actuation of the actuator 2132 configured to be contacted by the user may result in movement of both the first and the second linear gears 2140, 2150, which may result in one or more of: advancement of the elongate member 2110 or retraction of the elongate member 2110, and delivery of a fluid composition from the fluid reservoir 2162 to the eye.

The drive assembly 2130 may comprise one or more actuators 2132 (e.g., one, two, three, four, or more) configured to be contacted by a user, such as, for example, a rotatable element (e.g., wheel), slide, button, or the like, actuation of which (e.g., rotation, translation, depression) may advance and retract the elongate member 2110 and/or may deliver the fluid composition by way of the first linear gear 2140 and the second linear gear 2150. For example, as shown in FIG. 21, the drive assembly 2130 may include a first actuator configured to be contacted by a user 2132A and a second actuator configured to be contacted by a user 2132B. In some variations, such as that shown in FIG. 21, the first and second actuators 2132A, 2132B may both be rotatable wheels. The first actuator 2132A and the second actuator 2132B may each contact one or more gears 2134, 2136 to translate rotational movement of the actuators 2132A/2132B into linear movement of the linear gears 2140, 2150. In some variations, the drive assembly 2130 may comprise a clutch configured to selectively transfer the motion of the actuators 2132A, 2132B to the linear gears. In some variations, the clutch may be engaged by movement of an actuator in a first direction. When engaged, the clutch may move the first and second linear 2140, 2150 gears in opposite directions. The clutch may be disengaged by movement of the actuator in a second opposite direction. When disengaged, the clutch may move one of the first and second linear gears. In some variations, the clutch may decouple movement of a first linear gear 2140 from movement of a second linear gear 2150. In doing so, the second linear 2150 gear may advance or retract the elongate member without the first linear gear 2140 moving the displacement rod.

The fluid delivery device 2100 may further comprise a fluid assembly 2160 comprising a fluid reservoir 2162. The fluid reservoir 2162 may be at least partially contained within the housing 2104 of the handle 2102 and in this manner, may be configured to store a fluid composition for delivery to the eye. The fluid reservoir 2162 may be stationary or moveable within the handle 2102. The fluid reservoir 2162 may include a fluid reservoir connector 2164 configured to detachably couple to an external fluid delivery device. The external fluid delivery device may be configured to deliver a first volume of fluid to the fluid reservoir 2162, then detach from the fluid reservoir connector 2164 upon completion of delivering the volume of fluid to the fluid reservoir 2162.

The fluid assembly 2160 may further comprise components configured to interact with the fluid reservoir 2162 and thereby assist with delivering the fluid composition from the fluid reservoir 2162 to the eye. For example, the fluid assembly 2160 may comprise a plunger tube 2152 and a displacement rod 2142, each configured to move relative to (e.g., within) the fluid reservoir 2162 to deliver the fluid composition from the fluid reservoir 2162 to the eye. In some variations, the displacement rod 2142 may move in a first direction a single time, while the plunger tube 2152 may move in the first direction, a second direction opposite the first direction, and may cycle between the two directions numerous times. For example, in some variations, the displacement rod 2142 may only move into the fluid reservoir 2162 a single time but not out of the fluid reservoir, while the plunger tube 2152 may move into and out of the fluid reservoir 2162 any number of times.

Further, FIG. 21 depicts the elongate member 2110 disposed within a lumen 2109 of the cannula 2108. The elongate member 2110 may be slidably positioned within the cannula lumen 2109. When the elongate member 2110 is in a retracted position relative to the cannula 2108, a distal end of the elongate member 2110 may be located within (i.e., proximal to) a distal tip of the cannula 2108. When the elongate member 2110 is in an extended position relative to the cannula 2108, the distal end of the elongate member 2110 may be located outside of (i.e., distal to) the distal tip of the cannula 2108. The length of extension of the elongate member 2110 beyond the distal tip of the cannula 2108 may correspond to the distance around Schlemm's canal that may be traversed by the elongate member (e.g., in order to disrupt Schlemm's canal and/or surrounding trabeculocanalicular tissues, and/or to deliver a fluid composition). When a variation of the fluid delivery systems described herein is used to deliver a fluid composition, the length traversed by the elongate member 2110 may correspond to the length around Schlemm's canal to which the fluid composition is delivered. When a variation of the fluid delivery systems described herein is used to mechanically tear or cut the trabecular meshwork independent of fluid delivery, the length traversed by the elongate member 2110 may correspond to the length of trabecular meshwork that is cut or torn. In some variations, this length may be about 1 mm to about 50 mm. It should be appreciated, that in some variations, a same fluid delivery device (e.g., delivery device 1600A, 1600B, 2100) may be used to both deliver a fluid composition to Schlemm's canal and cut or tear the trabecular meshwork.

In some variations, the distal end of the elongate member 2110 may be configured as a curved tip, a compound curved tip, an atraumatic tip, an enlarged atraumatic tip, or the like, to help the elongate member 2110 be advanced through Schlemm's canal. In some of these variations, the distal end may comprise a blunt parasol-shaped atraumatic tip. In other variations, a distal portion of the elongate member 2110 may optionally include a disruptive component, e.g., a notch, hook, barb, a rough surface, or combination thereof, to disrupt the juxtatrabecular portion of Schlemm's canal or juxtatrabecular meshwork. One or more projections emanating from the elongate member 2110 may further disrupt the juxtatrabecular portion of Schlemm's canal or juxtatrabecular meshwork and thus increase permeability of aqueous humor through the trabecular meshwork into Schlemm's canal. In some instances, the elongate member 2110 may also deliver energy to the trabeculocanalicular tissues (e.g., ultrasonic energy, radiofrequency energy (e.g., for electrocautery, electroablation), electromagnetic radiation, light energy (e.g., via a fiber optic)).

As described above, the elongate member 2110 may include a conduit comprising a lumen. The elongate member 2110 may be configured to deliver a fluid composition. The fluid composition may travel through a lumen of the elongate member 2110 and may be delivered through one or more openings of the lumen. The fluid composition may be used to disrupt the trabecular meshwork and other surrounding tissues. The fluid composition may be delivered to the elongate member 2110 via the fluid assembly 2160. In some variations, the distal end of the elongate member 2110 may be configured or modified to aid delivery of the fluid composition into Schlemm's canal. For example, the distal end of the elongate member 2110 may comprise a cut out configured as a half tube. Additionally, or alternatively to an opening at the distal tip, the elongate member 2110 may optionally comprise a plurality of openings through its body (e.g., sidewall) that are spaced along the axial length of the elongate member 2110. In this variation, a fluid composition may be delivered from the fluid reservoir 2162 through the openings in the elongate member 2110 and into Schlemm's canal. This lateral ejection of fluid (e.g., a viscoelastic fluid) may in some instances enhance disruption of outflow tissues and enhance permeability to aqueous humor. It should be understood that the openings can be of any suitable number, size, and shape, and spaced along the axial length of the elongate member 2110 (including the distal tip) in any suitable manner. While described here with respect to the fluid delivery device 2110 of FIG. 21, it should be appreciated that the an elongate member having any of the features of the elongate member 2110 depicted in FIG. 21 may be used with any of the fluid delivery devices described herein, such as fluid delivery devices 1600A and 1600B depicted in FIGS. 16A and 16B respectively.

The fluid delivery devices herein may be used to perform one or more tissue disruption procedures, such as one or both of a trabeculotomy procedure and a fluid delivery procedure, as explained in more detail herein.

The delivery devices or components thereof described herein may in some variations be fully disposable. In other variations, a portion of one or both of the implant and fluid delivery devices herein may be reusable (e.g., non-patient contact materials, such as the handle 502, 1602), while a portion of a delivery device may be disposable (e.g., patient-contact materials, such as the cannula 508, 1608A/1608B, 2108 and the positioning tool or elongate member 2110). In yet other variations, the delivery devices (e.g., the implant and/or fluid delivery devices) described herein may be fully reusable.

### II. Kits

The delivery devices (e.g., implant delivery devices, fluid delivery devices) may be packaged together as a single kit, or may be packaged separately and sold together or separately for use together or separately. Kits may further comprise one or more devices configured to couple to an implant delivery device and/or a fluid delivery device, such as an external fluid device configured to couple to a fluid delivery device, and/or one or more additional implants for delivering via an implant delivery device. In some variations, the one or more additional implants may include a plurality of implants of different sizes. In some variations, the kits my further include one or more additional surgical tools, such as one or more of a scalpel, a tissue disruption tool, a cauterizer, and/or the like. Further, the kits herein may include instructions for use (IFU) of the delivery device(s) therein. For example, the IFU may guide a user through performing one or more procedures (e.g., trabeculotomy, fluid delivery, implantation, etc.) using one or more of the delivery devices therein.

In some variations, the kits and/or delivery devices described herein may be placed in specialized packaging. The packaging may be designed to protect the systems, and in particular, to protect the cannula. It may be desirable for the packaging to prevent contact between the distal tip of the cannula and any other object or surface. In order to do so, the packaging may comprise one or more elements configured to secure a delivery system to the packaging at one or more locations proximal to the distal tip of the cannula. Securing the delivery system at least two locations proximal to the distal tip of the cannula may be desirable to limit the ability of the delivery system to pivot relative to the packaging.

In one exemplary variation, the packaging may comprise a tray comprising a recess having a shape generally corresponding to the shape of the delivery system and comprising one or more pinch points configured to secure the delivery system at locations proximal to the cannula. In some variations, the kit may include one or more delivery systems, such as a plurality of the implant delivery systems described above, and the tray may include one or more corresponding recesses, such as a plurality of recesses shaped to fit an implant delivery device. In some variations, a kit may include an implant delivery device (e.g., at least one implant delivery device) and a fluid delivery device (e.g., at least one fluid delivery device), and the tray may include a first recess shaped to fit the implant delivery device and a second recess shaped to fit the fluid delivery device. In some variations, a kit may include an implant delivery device (e.g., at least one implant delivery device) and a fluid delivery device (e.g., at least one fluid delivery device), as well as a first tray having a first recess shaped to fit the implant delivery device, and a second, separate tray having a second recess shaped to fit the fluid delivery device.

It should be appreciated that the packaging may have other configurations that protect the distal tip of the cannula. For example, in another variation, the packaging may comprise a stiff planar sheet to which the delivery system may be attached in an orientation such that the cannula is not in contact with the planar sheet. The delivery system may be attached (e.g., via ties or other materials wrapped around the housing) at two or more points along the housing in order to prevent movement of the delivery system relative to the planar sheet. It may be desirable to protect the cannula on at least two sides; for example, a portion of the planar sheet near the cannula may be bent around the cannula to protect the cannula on at least two sides, or a second stiff planar sheet may be attached to the delivery system opposite the first planar sheet.

As described above, some kits described herein may comprise multiple delivery devices. For example, a kit may comprise 2, 3, 4, 5, or than 5 delivery systems. In some variations, the kit may comprise two of the same system, such that, for example, the first delivery system may be used in a first eye of the patient and the second delivery system may be used in the second eye of the patient. In other variations, the kit may comprise two different systems. For instance, the first delivery device may be configured to disrupt (at least a portion of) the trabecular meshwork using the elongate member. That is, the first delivery device may be configured to deliver a disruptive volume of fluid to (at least a portion of) the trabecular meshwork and/or to cut or tear (at least a portion of) the meshwork using the elongate member (e.g., a feature thereon, such as an external circumference of the elongate member). The second delivery device may be configured to deliver an implant to (at least a portion of) the trabecular meshwork. In some variations, the kit may include three delivery devices, such as a first delivery device (e.g., a fluid delivery device) for delivering a fluid composition to at least a first portion of trabecular meshwork, a second delivery device (e.g., a different fluid delivery device) for disrupting at least second, different a portion of the trabecular meshwork using the elongate member, and a third delivery device (e.g., an implant delivery device) for delivering an implant to at least a third, different portion of the canal. In some variations, the kit may include three delivery devices, such as a first delivery device (e.g., a fluid delivery device) for delivering a fluid composition to at least a first portion of Schlemm's canal, a second delivery device (e.g., an implant delivery device) for delivering an implant to at a second, different portion of the canal, and a third delivery device (e.g., a different implant delivery device) for delivering an implant to a third, different portion of the canal.

Some kits may comprise ocular implants in addition to one or more delivery systems as described herein. In general, an implant may be pre-installed within an implant delivery device such that it is provided within the cannula of the implant delivery devices. In some variations, the kits may include one or more extra implants for reloading an implant delivery device and/or implants of different sizes. In some variations, one or more of the delivery devices herein, and thus at least part of the kits herein, may be reusable. In other variations, none of the delivery devices, implants, or kits herein may be used for more than one procedure.

### III. Methods of Use

Methods for reducing intraocular pressure are provided below. The methods may be for one or more of delivering an ocular device (e.g., an implant) within the eye and disrupting ocular (e.g., trabeculocanalicular) tissues using a disruptive volume of fluid composition (e.g., viscoelastic fluid) and/or a disruptive tool. Methods for delivering implants, such as the implants described herein, may generally include using a delivery device to position an implant at least partially within Schlemm's canal. Implant-free tissue-disrupting methods may generally be achieved by using a delivery device to provide a force sufficient to disrupt trabeculocanalicular tissues. The methods may generally be single-handed, single-user controlled methods that are minimally invasive, e.g., they are tailored for an ab-interno procedure, which as previously mentioned, can be advantageous over the more invasive ab-externo approach. However, use of the ocular systems in an ab-externo method may be contemplated in some instances and thus, are not excluded here. The methods for delivering an ocular device or fluid, and/or for providing a disruptive force, may be used to treat glaucoma, pre-glaucoma, and/or ocular hypertension. When treating glaucoma, the methods may also be used in conjunction with a cataract surgery (before or after) using the same incision. The methods may be used alone or in combination to reduce intraocular and thus provide treatment for an ocular condition or disorder. Further, one or more additional treatment modalities may be used with the methods herein, including medication, incisional surgery, laser surgery, cryosurgery, other forms of surgery, and combinations thereof.

In some variations, the methods may generally include steps of creating an incision in the ocular wall (e.g., the sclera or cornea) that provides access to the anterior chamber of the eye, advancing a cannula of the delivery system through the incision and at least partially across the anterior chamber to the trabecular meshwork, and accessing Schlemm's canal with the cannula. The methods may also include the step of priming or flushing the system with fluid (e.g., to remove air from the system) and/or the step of irrigating the operative field to clear away blood or otherwise improve visualization of the field. The surgeon may first view the anterior chamber and trabecular meshwork (with underlying Schlemm's canal) using an operating microscope and a gonioscope or gonioprism. Using a 0.5 mm or greater corneal, limbal, or sclera incision, the surgeon may then gain access to the anterior chamber. A saline solution or viscoelastic composition may then be introduced into the anterior chamber to prevent its collapse. Here the saline solution or viscoelastic composition may be delivered through the cannula of the delivery system or by another mode, e.g., by infusion through an irrigating sleeve on the cannula. The surgeon, under direct microscopic visualization, may then advance the cannula of a delivery device through the incision towards the anterior chamber angle. When nearing the angle (and thus the trabecular meshwork), the surgeon may apply a gonioscope or gonioprism to the cornea to visualize the angle. The application of a viscous fluid (e.g., a viscoelastic composition as previously described) to the cornea and/or gonioscope or gonioprism may help to achieve good optical contact and negate total internal reflection thereby allowing visualization of the anterior chamber angle. As the surgeon visualizes the trabecular meshwork, the cannula may then be advanced so that the distal tip of the cannula is adjacent to the meshwork, in contact with the meshwork, or pierces the meshwork and to be communication with the lumen of Schlemm's canal.

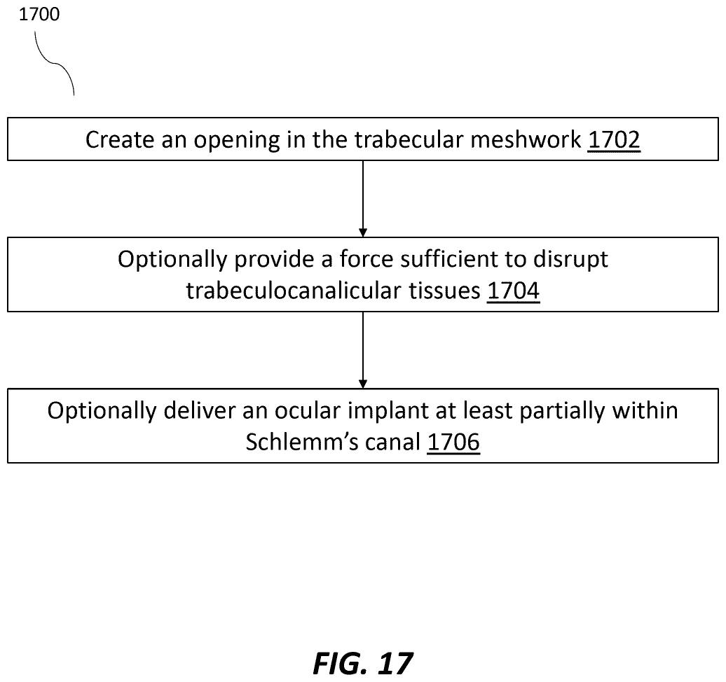

FIG. 17 depicts a flow diagram of a variation of a method 1700 for reducing intraocular pressure. First, the method 1700 may include creating 1702 an opening within the trabecular meshwork of the eye. In some variations, more than one opening may be formed. For example, in some variations, two, three, four, five, or more than five openings may be formed in the trabecular meshwork. The opening may provide point of entry for one or more delivery devices (e.g., a fluid delivery device and/or an implant delivery device, as described herein) to access the lumen of Schlemm's canal via the trabecular meshwork. In some variations, the step 1702 may be performed using a blade (e.g., a circular blade). In some variations, the step 1702 may be performed using a portion of a delivery device, such as using a distal tip of a cannula of a delivery device (e.g., a fluid delivery device) that has a cutting edge.

Second, the method 1700 may include optionally providing 1704 a force sufficient to disrupt trabeculocanalicular tissues, such as the tissues lining Schlemm's canal. The force may be provided by a fluid delivery device configured to provide a disruptive volume of fluid to at least a portion of the canal, and/or by a disruptive tool (e.g., of a delivery device). The delivery device and/or disruptive tool may be advanced through the opening in the trabecular meshwork and at least partially into Schlemm's canal to deliver the disruptive force. For example, an elongate member of a fluid delivery device may be advanced into Schlemm's canal to deliver a viscoelastic fluid and/or to provide a disruptive force to the canal. The step 1704 may be performed any suitable number of times. For example, in some variations, the step 1704 may be repeated at least twice (e.g., two or more times, a plurality of times) to both provide a combination of a disruptive volume of fluid within the canal and a disruptive force to the canal. In some variations, the step 1704 may be repeated two or more times to only to provide a disruptive volume of fluid within the canal, which is referred to herein as a fluid delivery (e.g., viscodilation) procedure. For example, the step 1704 may include delivering a volume of viscoelastic fluid to a plurality of portions (such as two or more portions) of the canal, or delivering the fluid to a single portion of the canal multiple times. Alternatively, in some variations, the step 1704 may be repeated two or more times to only provide a disruptive force to the canal using a tool, which is referred to herein as a trabeculotomy procedure. For example, the step 1704 may include using a tool to deliver a disruptive force to a plurality of portions (such as two or more portions) of the canal. Generally, the step 1704 may be performed prior to optional step 1706, delivering an ocular implant, as is described below. In some variations, delivering an ocular implant at least partially within Schlemm's canal, step 1704, alone may effectively reduce intraocular pressure. In some variations, step 1704 may not be performed. The fluid delivery and trabeculotomy procedures of step 1704 are described in more detail below, followed by implantation procedure of step 1706.

### Fluid Delivery

A fluid delivery step may be performed using the fluid delivery devices described herein. The procedure may include piercing the trabecular meshwork with the distal tip of a cannula to enter Schlemm's canal. Additionally, fluid delivery may generally include advancing the slidable elongate member disposed within the cannula lumen into and around the canal under gonioscopic visualization. The elongate member may be advanced any suitable amount and direction about the canal. For example, the elongate member may be advanced about 1 degree to about 360 degrees about the canal, about 10 degrees to about 360 degrees about the canal, about 150 to about 210 degrees about the canal, or any suitable distance, about 360 degrees about the canal, about 270 degrees about the canal, about 180 degrees about the canal, about 120 degrees about the canal, about 90 degrees about the canal, about 60 degrees about the canal, about 30 degrees about the canal, or about 5 degrees about the canal. In some variations, the elongate member may be advanced in two steps, e.g., first in a clockwise direction (e.g., about 180 degrees, about 90 degrees, etc.) and second in a counterclockwise direction (e.g., about 180 degrees, about 90 degrees, etc.) about the canal (e.g., to thereby achieve a 360- or 180-degree ab-interno viscocanalostomy or canaloplasty). In some variations, the elongate member may be advanced in one step (e.g., about 90 degrees in a clockwise or counterclockwise direction, about 180 degrees in a clockwise or counterclockwise direction, about 270 degrees in a clockwise or counterclockwise direction, about 360 degrees in a clockwise or counterclockwise direction)) about the canal to thereby achieve a corresponding degree ab-interno viscocanalostomy or canaloplasty. Advancing the elongate member around Schlemm's canal includes using the drive assembly to advance the elongate member around Schlemm's canal. In some embodiments, using the drive assembly to advance the elongate member around Schlemm's canal includes using the one or more actuators including the one or more wheels, slides, or buttons to advance or retract the elongate member around Schlemm's canal.

Fluid may be injected into the canal upon advancement or retraction of the elongate member. Once the slidable elongate member has been positioned within the canal, a fluid composition, e.g., a viscoelastic solution, may be continuously or intermittently delivered through the lumen of the elongate member. The fluid composition may exit the lumen of the elongate member through its distal end (e.g., the through the distal tip), or through openings or fenestrations provided along its shaft, or a combination of both. The openings or fenestrations may be spaced along the axial length of the elongate member in any suitable manner, e.g., symmetrically or asymmetrically along its length. Other substances such as drugs, air, or gas may delivered be in the same manner if desired. In some variations, retracting the elongate member may include using the first actuator to retract the elongate member and deliver fluid to Schlemm's canal simultaneously, while in other variations, the retraction of the elongate member and delivery of fluid may be entirely decoupled. Accordingly, in some variations, the method may include delivering fluid to Schlemm's canal, then retracting the elongate member, or vice versa. This combination of delivering fluid and retracting the elongate member (and/or advancing the elongate member) may be completed any suitable number of times during a procedure. It should be appreciated that the elongate member may be advanced and/or retracted any desirable amount around Schlemm's canal to disrupt the tissues and appropriately place the elongate member for fluid delivery. Any suitable number of actuators may be used to advance the elongate member, retract the elongate member, and deliver fluid, including using a single actuator for all, individual actuators for all, or a combination thereof. For example, in some variations, retracting the elongate member may include using a second separate actuators to retract the elongate member and to deliver fluid to Schlemm's canal.

The fluid composition may be delivered via the elongate member. The elongate member and/or fluid delivery may dilate Schlemm's canal, and fluid delivery may additionally dilate the collector channels. The entire length of Schlemm's canal or a portion thereof may be dilated by the fluid. For example, at least 75%, at least 50%, at least 25%, at least 10% of the canal, or at least 1% of the canal may be dilated. As another example, an area of about 1 degree to about 360 degrees of the canal may be dilated by the fluid, such as an area of about 10 degrees to about 350 degrees, about 50 degrees to about 310 degrees, about 90 degrees to about 290 degrees, about 110 degrees to about 250 degrees, about 150 degrees to about 210 degrees, to 150 degrees to about 190 degrees, about 45 degrees, about 90 degrees, about 135 degrees, about 180 degrees, about 225 degrees, about 270 degrees, about 315 degrees, or about 360 degrees.

The viscoelastic fluid may be delivered while advancing the elongate member of a single-handed, single-user controlled device from Schlemm's canal in the clockwise direction, counterclockwise direction, or both, or during withdrawal of the elongate member from Schlemm's canal. As previously stated, the viscoelastic fluid may be delivered to disrupt Schlemm's canal and surrounding trabeculocanalicular tissues. For example, the delivered viscoelastic fluid may cause disruption by dilating Schlemm's canal, increasing the porosity of the trabecular meshwork, stretching the trabecular meshwork, forming microtears or perforations in juxtacanalicular tissue, removing septae from Schlemm's canal, dilating collector channels, or a combination thereof. The elongate member may be loaded with the viscoelastic fluid at the start of an ocular procedure so that the fluid can be delivered by a single device. This is in contrast to other systems that use forceps or other advancement tool to advance a fluid delivery catheter into Schlemm's canal and/or devices containing viscoelastic fluid that are separate or independent from a delivery catheter or catheter advancement tool, and which require connection to the delivery catheter or catheter advancement tool during a procedure by an assistant while the delivery catheter or catheter advancement tool is held by the surgeon.

In some variations, the slidable elongate member may be repositioned by retraction or repeated advancement and retraction. In some variations of the method, the same or different incision may be used, but the delivery device cannula is employed to access and dilate Schlemm's canal from a different direction (e.g., counterclockwise instead of clockwise). Once a sufficient amount of fluid has been delivered, the surgeon may retract the slidable elongate member into the cannula and remove the delivery system from the eye. It should be appreciated that the cannulas described here may be specifically manufactured to comprise a dual-surface configuration at the distal tip (i.e., sharp and smooth surfaces), which may allow the elongate member to be advanced, repositioned, and/or retracted without severing it on the distal tip of the cannula.

In some variations of the ab-interno method, the fluid composition may be delivered simultaneously with retraction of the elongate member (i.e., the fluid compositions may be delivered in a manner where retraction of a system component allows advancement of the fluid out of the system cannula). It should be understood that the delivery systems may be configured so that the fluid compositions are delivered continuously, passively, automatically, or actively by the surgeon. The fluid compositions may also be delivered to the canal independent of the gear shaft movement with a pump or auxiliary plunger. In some variations, retraction of the elongate member may correspond to a fixed volume of fluid composition being delivered via the lumen of the elongate member. The fluid composition may be delivered via the distal opening of the lumen of the elongate member as it is retracted, and thus, the fluid may be evenly delivered throughout the portion of the canal through which the elongate member was advanced.

The fluid compositions that may be delivered by the systems described herein include but are not limited to saline and viscoelastic fluids. The viscoelastic fluids may comprise hyaluronic acid, chondroitin sulfate, cellulose, derivatives or mixtures thereof, or solutions thereof. In one variation, the viscoelastic fluid comprises sodium hyaluronate. In another variation, the viscoelastic composition may further include a drug. For example, the viscoelastic composition may include a drug suitable for treating glaucoma, reducing or lowering intraocular pressure, reducing inflammation, fibrosis neovascularization or scarring, and/or preventing infection. For example, in some variations, the viscoelastic composition may include the therapeutic agents described herein, such as but not limited to Rho kinase (ROCK) inhibitors and agents for gene therapy, DNA, RNA, or stem cell-based approaches. In some variations, the fluid compositions may also be delivered to treat various medical conditions of the eye, including but not limited to, glaucoma, pre-glaucoma, anterior or posterior segment neovascularization diseases, anterior or posterior segment inflammatory diseases, ocular hypertension, uveitis, age-related macular degeneration, diabetic retinopathy, genetic eye disorders, complications of cataract surgery, vascular occlusions, vascular disease, or inflammatory disease. The viscoelastic composition may also include agents that aid with visualization of the viscoelastic composition. For example, dyes such as but not limited to fluorescein, trypan blue, or indocyanine green may be included. In some variations, a fluorescent compound or bioluminescent compound is included in the viscoelastic composition to help with its visualization. In other variations, the system may deliver the drug alone, without the viscoelastic composition. In this case, the drug may be loaded onto or into a sustained release biodegradable polymer that elutes drug over a period of weeks, months, or years. It is also contemplated that air or a gas could be delivered with the systems, as described herein.

Exemplary volumes of viscoelastic fluid that may be delivered may in some instances be about 1 μl to about 200 μl, or in some instances be about 1 μl to about 100 μl. In some instances, sufficient volumes to provide a disruptive force may range from about 1 μl to about 50 μl, from about 1 μl to about 30 μl, or from about 2 μl to about 16 μl. In one variation, a volume of about 4 μl is sufficient to disrupt Schlemm's canal and/or the surrounding tissues. In other variations, the volume of viscoelastic fluid sufficient to disrupt trabeculocanalicular tissues may be about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 25 μl, about 30 μl, about 35 μl, about 40 μl, about 45 μl, or about 50 μl.

### Trabeculotomy

A trabeculectomy procedure may generally include providing a disruptive force to trabeculocanalicular tissues, such as tearing or cutting the trabecular meshwork of Schlemm's canal. The force may be delivered via a disruptive tool such as a tool having notches, hooks, barbs, balloons, or combinations thereof. In other instances, the disruptive tools may not comprise disruptive components on their distal portion and may instead have atraumatic blunt distal portions such as parasol or dome shaped distal portions.

A trabeculectomy may be performed using the delivery devices described herein, such as with the elongate member of the fluid delivery devices described herein. The outer diameter of the elongate member or tool may be variously sized for disruption of tissues, analogous to how fluid volumes may be varied to vary the level of disruption. For example, an elongate member or tool having an outer diameter ranging from about 50 to about 100 microns may be advanced through the canal to slightly dilate the canal and break or remove septae obstructing circumferential canalicular flow. An elongate member or tool having an outer diameter ranging from about 100 to 200 microns may be employed to perform the foregoing and may also to begin to stretch the trabecular meshwork and juxtacanalicular tissues. An elongate member or tool having an outer diameter ranging from about 200 to about 300 microns may be able to perform the above but may also create microtears in the trabecular meshwork and juxtacanalicular tissues and may maximally dilate the collector channels. An elongate member or tool having an outer diameter ranging from about 300 to about 500 microns may maximally disrupt the tissues and may create tears or perforations all along the trabecular meshwork and juxtacanalicular tissues. In some variations, the elongate member may have a non-uniform outer diameter. For example, the elongate member may have a tapered outer diameter, such that the outer diameter increases from the distal to proximal end.

To perform the trabeculotomy, the elongate member (or other tool) may be advanced out from the tip of the cannula and into the canal about a 30 degree arc of the canal (e.g., advanced about 3 to 4 mm out of the cannula), advanced about a 60 degree arc of the canal (e.g., advanced about 6 to 8 mm out of the cannula), advanced about a 90 degree arc of the canal (e.g., advanced about 10 mm out of the cannula), advanced about a 120 arc of the canal (e.g., advanced about 15 mm out of the cannula), advanced about a 180 degree arc of the canal (e.g., advanced about 20 mm out of the cannula), or advanced about a full 360 degrees of the canal (e.g., advanced about 36 to 40 mm out of the cannula), for maximal intraocular pressure reduction. In some variations, the elongate member may be advanced between about a 5-degree arc of Schlemm's canal and about a 360-degree arc. In some variations, the methods may include advancement of the elongate member (or tool) about a 360-degree arc of Schlemm's canal, about a 270-degree arc of Schlemm's canal, about a 120-degree arc of Schlemm's canal, about a 180-degree arc of Schlemm's canal, or about a 90-degree arc of Schlemm's canal. In yet further variations, advancement of the elongate member (or a tool) may be about a 0-to-5-degree arc of Schlemm's canal, about a 30-degree arc of Schlemm's canal, or about a 60-degree arc of Schlemm's canal. It may be beneficial to advance the elongate member in both clockwise and counterclockwise directions about a 180-degree arc of Schlemm's canal from a single access point (e.g., the opening created in step 1702 of method 1700) in the canal. In other variations, the elongate member may be advanced in a single (clockwise or counterclockwise) direction about 360 degrees of Schlemm's canal from a single access point in the canal.

Depending on factors such as the type or severity of the condition being treated, the disruptive force may be generated to partially or completely destroy and/or remove the trabecular meshwork and may be adjusted by varying the tool configuration. In some methods, the trabecular meshwork may be disrupted during advancement of the slidable elongate member. Customizing a body segment of the elongate member proximal to the tip with one or more notches, barbs, or balloons that catch the meshwork as the distal tip is being guided and advanced along Schlemm's canal may also be used, thereby disrupting, partially tearing, fully tearing, and/or removing trabecular meshwork upon advancement. Additionally, an implant with edges specifically designed to cut the meshwork may be used.

In yet other methods, the trabecular meshwork may be disrupted during retraction of the slidable elongate member. The methods for disrupting tissues may involve customizing the system (e.g., the elongate member, any catheters or wires, probe tips, etc.) to catch or grasp the meshwork upon retraction after advancement through the canal. This may be done using a wire with a bent tip, hook, notch, or barb on its end that is advanced through the lumen of the catheter that then snags the meshwork upon retraction, tearing it along its length or removing it altogether, or solely with a metal or polymer wire or suture (no catheter) whose tip (and/or body) is hooked, notched, or barbed in such a way that it can be advanced into Schlemm's canal without tearing the meshwork but snags the meshwork upon retraction, tearing the meshwork and/or removing it completely. The elongate member may be provided with a disruptive tool, e.g., a sharp-edged element, that can cut or tear the trabecular meshwork while being retracted into the cannula, which is held stationary. Exemplary sharp-edged elements may be a hook, wire, or any other suitable shape memory component that can extend from the cannula to tear, cut, or remove trabecular meshwork.

Another method for disrupting tissues may include using oversized elongate members (e.g., having an outside diameter of 300-500 microns) to tear the meshwork upon delivery, or inflating or expanding the elongate member once it has been fully advanced into Schlemm's canal to stretch, disrupt, rupture, or fully tear the meshwork. For example, a catheter/elongate member, probe, or wire (with or without a lumen) whose tip is 200-250 microns in outer diameter, but having a shaft that begins to flare outwards after 3 clock hours of Schlemm's canal (i.e., at about the 5 or 10 mm mark on the catheter/elongate member) up to about 300, up to about 400, or up to about 500 microns, may be used, so that as the tip advances comfortably within Schlemm's canal, the enlarged shaft trails behind and ruptures the trabecular meshwork as it is advanced.

In another method, cutting, destruction, removal, or the like of the trabecular meshwork may be accomplished by removing the cannula from the eye while leaving the elongate member in the canal, thereby tearing through the meshwork. For example, a cannula may be inserted into the anterior chamber and Schlemm's canal, and a tool (e.g., a slidable elongate member) may be advanced within the canal. The cannula may be removed from the anterior chamber without retracting the elongate member. This action by itself may tear the trabecular meshwork. As the cannula is removed from the anterior chamber, the elongate member may begin tearing the trabecular meshwork from the point at which the cannula was inserted into Schlemm's canal and may continue tearing around the trabecular meshwork toward the distal end of the elongate member.

The fluid delivery and trabeculotomy procedures may carried out separately, or may be combined into a single procedure. For example, in some instances a portion (e.g., half) of Schlemm's canal may be dilated (using a fluid composition, for example), and the trabecular meshwork of the same or a different portion of Schlemm's canal may be torn or cut (or removed), within the same eye. As another example, all of Schlemm's canal may be dilated, and then all or a portion of the trabecular meshwork may subsequently be torn or cut.

In some variations, the fluid delivery and trabeculotomy procedures may be performed using a single delivery device, such as by one of the fluid delivery devices described herein. For example, the elongate member of a fluid delivery device may be used to deliver a fluid composition to a portion of Schlemm's canal (e.g., about a 180-degree arc of the canal, about a 90-degree arc of the canal) as described herein, and the same fluid delivery device may be used before or after the fluid delivery to tear or cut the trabecular meshwork in a same or different portion of the canal. In other variations, the fluid delivery and trabeculotomy procedures may be performed using different delivery systems (e.g., the dilation may be performed using a fluid delivery device, and the tearing or cutting may be performed using a delivery device or system that is not configured to deliver a fluid). In some variations, dilation may be performed in one eye of a patient, while the trabecular meshwork may be torn or cut in the other eye of the patient.

Turning back to FIG. 17, the method 1700 may include optionally delivering 1706 an ocular implant at least partially within Schlemm's canal. Implants such as those described herein may be suitable for maintaining the patency of Schlemm's canal and/or improving outflow of aqueous humor without substantially interfering with fluid flow across and along the canal. A suitable implant for use in the implantation step 1706 may be biased to have a curve corresponding to a curvature of Schlemm's canal. As described above, in some variations, the curve may have an angular span of between about 10 degrees and about 360 degrees (e.g., about 90 degrees), and thus the implant may occupy an arc of the canal of between about 10 degrees and about 360 degrees. The implantation step 1706 may be repeated any suitable number of times, such as more than one time to position more than one implant into an eye. In some variations, two or more implants may be placed at least partially within the canal. For example, the step 1706 may include implanting two or more implants circumferentially adjacent or circumferentially opposed to each other (centered about 180 degrees apart around the circumference of Schlemm's canal) within the canal. A same or different delivery device, such as an implant delivery device described herein, may be used to position more than one implant at least partially within Schlemm's canal. An implant may be delivered through a implant delivery device (e.g., a cannula of an implant delivery device) via the opening created in step 1702. The implant delivery device may be used to deliver an implant in a clockwise direction in an eye or in a counterclockwise position in the eye. It should be appreciated that in some variations, the implant delivery devices described herein may be configured to be used in a particular configuration (e.g., with a single side up, only in a clockwise direction, only in a counterclockwise direction, etc.).

Generally, the implantation step 1706 may be performed after the tissue disruption step 1704 (e.g., after one or more repetitions of the optional step 1704). Because delivering the implant includes placing the implant within the lumen of Schlemm's canal, the implantation step 1706 may not occur along a portion of Schlemm's canal that was cut or torn via step 1704 (e.g., if the meshwork of the canal was removed via a trabeculotomy). That is, successfully delivering the implant within the canal may depend on the ability of at least a portion of the canal to support the implant therein. Otherwise, one or more implantation steps 1706 (e.g., for delivering one or more implants) may occur within portions of the canal that have not been removed, or within portions of the canal that have been dilated via a fluid delivery step 1704. In some variations, the implantation step 1704 may occur after a tissue disruption step 1704 where tissue was disrupted (e.g., via fluid and/or a disruptive tool) in a first portion of Schlemm's canal, and an implant used during the implantation step 1706 may be delivered to a second, different portion of the canal. In some variations, the implantation step 1704 may occur directly after the opening step 1702, and the disruption step 1704 may not be performed. In some variations, the implantation step 1706 may not be performed.

### Implantation

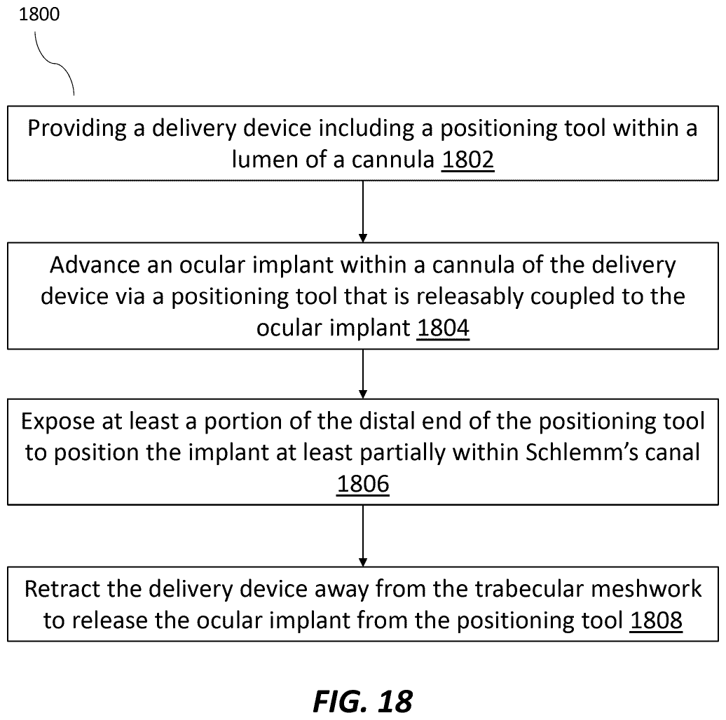

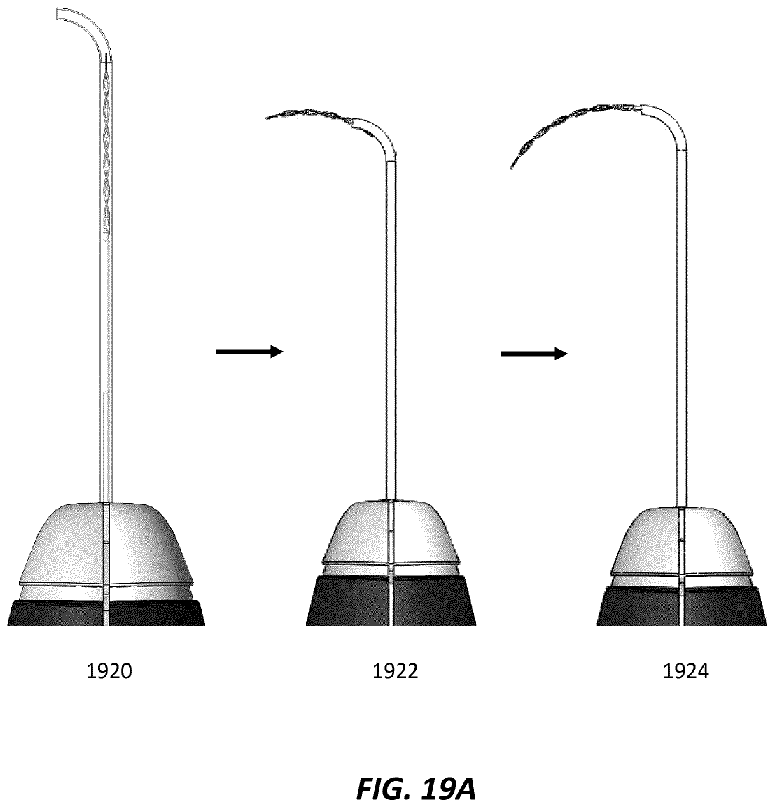

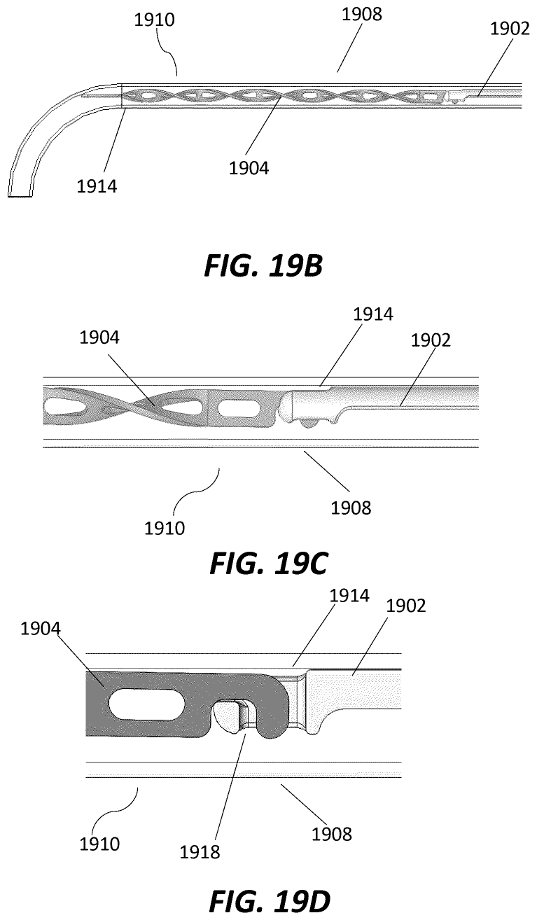

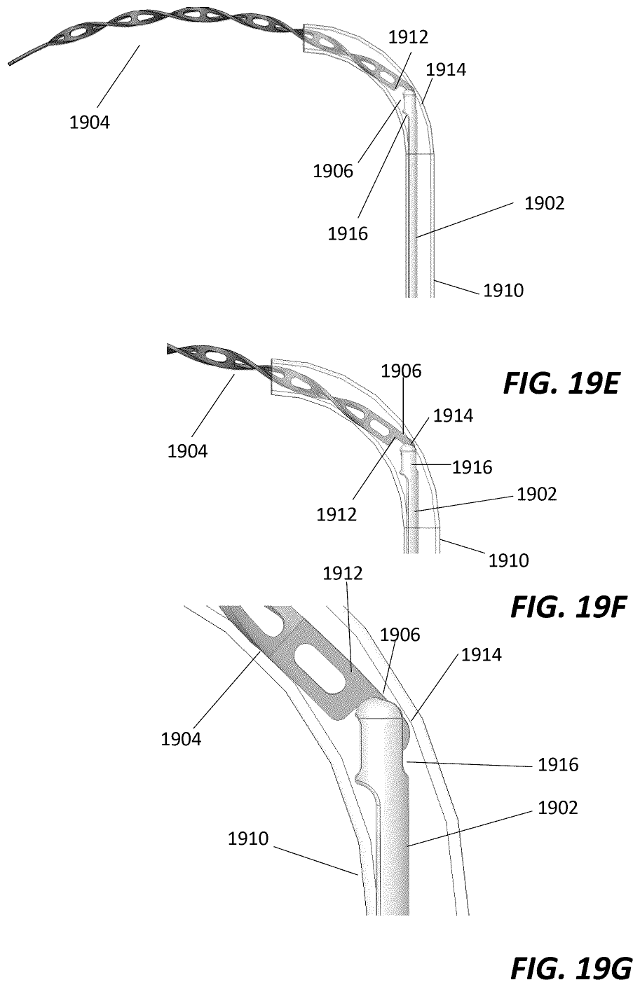

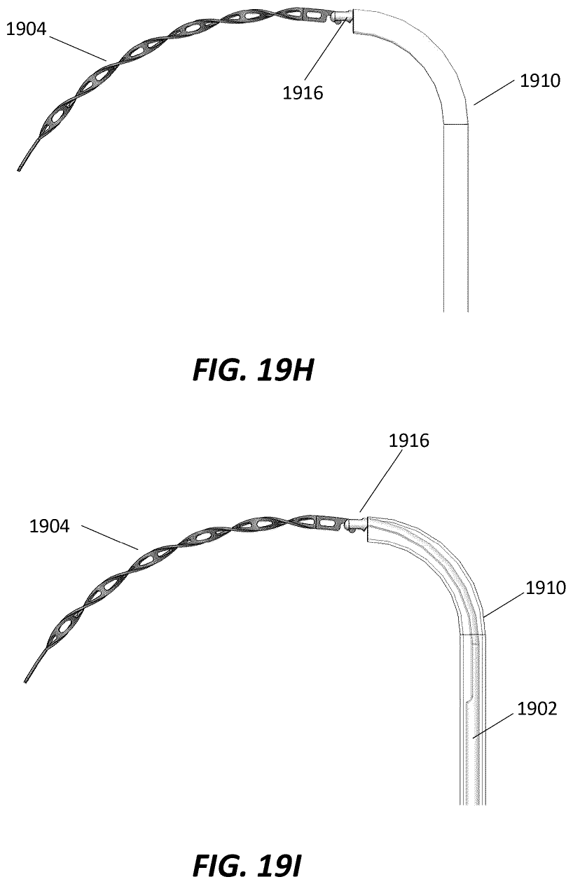

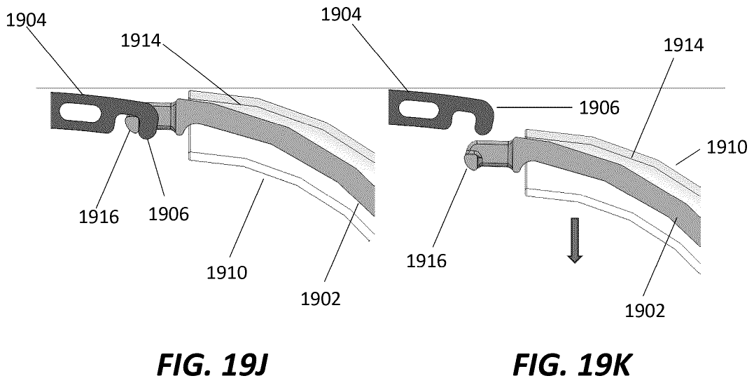

To expand on the step 1706 of the method 1700, FIG. 18 provides a flow chart of a variation of a method 1800 for delivering an ocular implant to Schlemm's canal. Additionally, FIGS. 19A-19K provide a variation of system for treating ocular conditions for performing a method for delivering an ocular implant to Schlemm's canal, such as a variation of the method 1800. As depicted in FIG. 19A, the system may be actuated from an initial stored configuration 1920, to a transitional mid-deployment state 1922, and subsequently to a deployed state 1924 during delivery of an ocular implant to Schlemm's canal.

Referring to FIG. 18, the method 1800 may first include providing 1802 a implant delivery device comprising a cannula that houses a positioning tool therein, where the positioning tool is releasably coupled to an ocular implant (e.g., providing the implant delivery devices described herein). At this step, the implant delivery device may be in an initial stored configuration. FIGS. 19B-19D each depict a variation of an initial configuration of the implant delivery device and implant therein. As shown, the positioning tool 1902 and implant 1904 are coupled within a straight proximal portion 1908 of the cannula 1910. Additionally, a back surface of the proximal portion 1912 of the implant 1904 is contacting an inner cannula surface 1914 of the cannula 1910.

The method 1800 may then include advancing an ocular implant within a cannula of the implant delivery device via the positioning tool 1804, where the positioning tool is releasably coupled to the ocular implant. For example, an engagement feature at a distal end of the positioning tool may be releasably coupled to a complementary engagement feature at a proximal end of the implant. Generally, the step 1804 may include actuating the positioning tool using one or more actuators of a delivery assembly. The step 1804 may also include positioning the implant delivery device adjacent to, or against, the opening of the trabecular meshwork. In some variations, the step 1804 may involve a distal portion of the implant being advanced out of the distal tip of the cannula while a proximal portion of the implant is still being advanced within the lumen of the cannula. Accordingly, the step 1804 may include advancing the implant within the anterior chamber, where at least a portion of the implant may be advanced through the trabecular meshwork and, in some cases, within Schlemm's canal. Moreover, as described above, the step 1804 may include contacting an inner surface of the cannula with the implant (e.g., with at least a portion of the engagement mechanism of the implant) during at least a portion of the delivery procedure. The contact may be maintained within the lumen of the cannula via the positioning tool (e.g., via the engagement mechanism of the positioning tool). Accordingly, the step 1804 may include controlling advancement of the implant via the positioning tool as long as the proximal portion of the implant (having the engagement mechanism) is within the lumen of the cannula. FIGS. 19E-19G illustrate a variation of this feature. As shown, the engagement mechanism 1906 of the implant 1904 may be held between the cannula surface 1914 (e.g., against an inner surface of a top cross-sectional arc of the cannula) and the engagement mechanism 1916 of the positioning tool 1902 such that the proximal portion 1912 of the implant 1904 and the distal portion of the positioning tool 1902 are interfacing within the cannula 1910. The opposing curvatures of the cannula 1910 (e.g., a first curved distal portion thereof) and the positioning tool 1902 (e.g., a second curved distal portion thereof) may cause the engagement mechanism 1906 to be held against the cannula surface 1914. In some variations, a portion of the positioning tool 1902 (e.g., at least a portion of the engagement mechanism 1916) may also contact the cannula surface 1914 during at least a portion of the step 1804 of the method 1800.

Next, the method 1800 may include exposing 1806 at least a portion of a distal end of the positioning tool. That is, step 1806 may include advancing at least a portion of the distal end of the positioning tool, such as the portion comprising the engagement mechanism, out of the cannula lumen (e.g., through a distal exit of the cannula). Like step 1804, the step 1806 may include actuating the positioning tool using one or more actuators of a delivery assembly. This step is illustrated in FIGS. 19H-19I, where the engagement mechanism 1916 of the positioning tool 1902 not within the cannula 1910. As shown, in some variations, the implant 1904 and positioning tool 1902 may remain coupled upon advancement of the engagement mechanism 1916 of the positioning tool out of the cannula 1910. In other variations, the implant 1904 and positioning tool 1902 may automatically release after exposing the engagement mechanism 1916 from the cannula 1910, and the following step 1808 of the method 1800 (described below) may not be necessary to deliver an implant into Schlemm's canal.

Finally, the method 1800 may include retracting 1808 the implant delivery device (e.g., the positioning tool of the implant delivery device) away from the trabecular meshwork to release the ocular implant from the positioning tool. A user may perform this step by simply moving the positioning tool and/or the entire implant delivery device away (e.g., retracting or otherwise moving the positioning tool and/or the device via the handle and/or the actuator(s)) from the implantation site (e.g., in a direction opposite to the opening within the trabecular meshwork). As described herein throughout, the engagement between the implant and the positioning tool may be a loose engagement (e.g., a clearance fit or transition fit) maintained in the cannula via the contact between the inner cannula wall and the implant caused by the opposing curves of the cannula and the positioning tool. The loose coupling may therefore have a low coefficient of friction. Accordingly, when the implant experiences a force that is greater than frictional force between the positioning tool and the implant, the implant may react more to the larger force. As an example, when at least a portion of the implant is within the trabecular meshwork, such as when the implant is being advanced out of the implant delivery device and into Schlemm's canal, the tissue of the trabecular meshwork exerts a force (e.g., an upward normal force) on the implant. When the engagement mechanisms described herein are subsequently exposed from the cannula, the force that was coupling the positioning tool and the cannula (e.g., the downward normal force of the interior of the cannula against the proximal portion of the implant and the upward normal force of the engagement mechanism of the positioning tool against the proximal portion of the implant) may no longer be applied. Thus, the force of the trabecular meshwork against the implant may overcome the smaller force caused only by minor friction between the positioning tool and the implant (due to a loose coupling). The step 1808 is visualized in FIGS. 19J-19K. In both FIGS. 19J and 19K, at least a distal portion of the implant 1902 (not shown) may be within the trabecular meshwork. In FIG. 19J depicts another perspective of FIGS. 19H and 19I, where the engagement mechanism 1916 of the positioning tool 1902 is not within the cannula 1910, and the implant 1904 and the positioning tool 1902 remain coupled. FIG. 19K shows that, in response to a force that is greater than the force of friction between the engagement mechanisms 1906, 1916, the engagement mechanism 1916 may be released from the engagement mechanism 1906 of the implant 1904. The force may be applied to the positioning tool 1902 via a portion of the implant delivery device. For example, as shown in FIG. 19K, the force may be a downward force (e.g., away from the proximal portion 1912 of the implant 1904) that is initiated manually by a user and translated to the positioning tool 1902 by the inner cannula surface 1914. In some variations, the force may be a backward force (e.g., longitudinally proximal relative to the implant 1904), or may be combination of a downward and backward force (e.g., diagonal).

In some variations, the method 1800 may optionally include using the implant delivery device (e.g., one or both of the positioning tool and the cannula) to reorient or reposition the implant within Schlemm's canal. For example, the positioning tool and/or and the cannula may be used to tap the implant into Schlemm's canal, and/or to manipulate a portion of the implant remaining in the anterior chamber or within Schlemm's canal so as to rotate the implant within the canal. As another example, the positioning tool may be used to recapture the implant following step 1808. Recapturing the implant may offer a user more precise control over positioning the implant within the canal, which may improve the accuracy of the implantation. In some variations, a recaptured implant may be retracted back into the cannula (e.g., to regain control over the implant via the positioning tool or to completely remove the implant from the eye). Additionally, or alternatively, the method 1800 may include anchoring the implant to tissue surrounding Schlemm's canal. Anchoring the implant to tissue may be accomplished in a variety of ways (e.g., by suturing, application of adhesives, installation of hooks, clips, or the like, or combinations thereof). In certain variations, the method 1800 may comprise selecting the size (e.g., arc length or angular span) of the implant such that the implant fits securely into the canal by a friction fit.

The procedures described above with respect to the method 1700, including goniotomy, fluid delivery, trabeculotomy, and implantation, may be used in any suitable combination. In some variations, one or more combinations of the aforementioned procedures may be used to reduce intraocular pressure in one or both eyes of a patient. Generally, a goniotomy may be performed prior to or simultaneously with one or more of a fluid delivery, trabeculotomy, or implantation procedure. For example, a tool may be used to create an opening in the trabecular meshwork, and one or more additional procedures may subsequently be performed to reduce intraocular pressure, or an opening may be created via a distal tip of a delivery device (e.g., a fluid delivery device) as one or more of the remaining procedures are being performed. In some variations, two or more procedures may be performed via the same opening within the trabecular meshwork (e.g., via the same incision made during goniotomy). In some variations, two or more procedures may be performed via two or more different openings within the trabecular meshwork. As will be described below, a combination of procedures may be achieved by applying a first procedure in a first direction of the canal (e.g., in a clockwise or counterclockwise direction), and then applying at least a second procedure in a second direction of the canal (e.g., in a counterclockwise or clockwise direction). For example, the first procedure may be a fluid delivery procedure or a trabeculotomy procedure that is performed in a clockwise direction, and the second procedure may be an implantation procedure that is applied in a counterclockwise direction. In some variations, one or more of the procedures (e.g., a fluid delivery procedure and a trabeculotomy procedure) may be applied in a same direction within the canal. Moreover, in some variations, one or more procedures may be performed a plurality of times (e.g., two times, three times, four times, five times, or more than five times).

In some variations, a first portion of Schlemm's canal may be viscodilated, and a second (same, different, or overlapping) portion of the canal may receive an ocular implant therein. For example, the fluid delivery may occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal), and the implantation may also occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal). In some variations, the fluid delivery may be performed in a first direction (e.g., clockwise or counterclockwise through the canal), and the implantation may be performed in the same first direction. In alternative variations, the fluid delivery may be performed in a first direction (e.g., clockwise or counterclockwise through the canal), and the implantation may be performed in a second, different direction (e.g., counterclockwise or clockwise). The implantation may occur prior to or following the fluid delivery. As another example, a first portion of Schlemm's canal may be viscodilated, and a second (same, different, or overlapping) portion of the trabecular meshwork of the canal may be cut, torn, or removed. For example, the fluid delivery may occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal), and the trabeculotomy may also occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal). In some variations, a first portion of the trabecular meshwork may be cut, torn, or removed, and a second portion the canal still retaining trabecular meshwork (e.g., the trabecular meshwork was torn therefrom) may be viscodilated. In some variations, the fluid delivery may be performed in a first direction (e.g., clockwise or counterclockwise), and the trabeculotomy may be performed in the same first direction. In alternative variations, the fluid delivery may be performed in the first direction (e.g., clockwise or counterclockwise), and the trabeculotomy may be performed in a second, different direction (e.g., counterclockwise or clockwise). The trabeculotomy may be performed prior to, following, or simultaneously with the fluid delivery. As another example, a first portion of the trabecular meshwork may be cut, torn, or removed, and a second, different portion of the canal may receive an implant therein. For example, the trabeculotomy may occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal), and the implantation may also occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal). In some variations, a first portion of the trabecular meshwork may be cut, torn, or removed, and a second portion the canal still retaining trabecular meshwork may receive the implant. In some variations, the trabeculotomy may be performed in a first direction (e.g., clockwise or counterclockwise), and the implantation may be performed in the same first direction but in a different location. In alternative variations, the trabeculotomy may be performed in the first direction (e.g., clockwise or counterclockwise), and the implantation may be performed in a second, different direction (e.g., counterclockwise or clockwise). The implantation may be performed prior to or following trabeculotomy. As yet another example, a first portion of the canal may be viscodilated, a second (same, different, or overlapping) portion of the trabecular meshwork of the canal may be cut, torn, or removed, and a third (same, different, or overlapping with respect to the first portion, and/or different with respect to the second portion) portion of the canal may receive an implant therein. For example, the fluid delivery may occur within about 10 degrees and about 360 degrees of Schlemm's canal, the trabeculotomy may also occur within about 10 degrees and about 360 degrees of Schlemm's canal, and the implantation may occur within about 10 degrees and about 360 degrees of the canal. The fluid delivery and the implantation may generally be performed within portions of the canal still retaining trabecular meshwork. The fluid delivery may be performed in a first direction (e.g., clockwise or counterclockwise), the trabeculotomy may be performed in the first direction or in a second, different direction, and the implantation may be performed in the first direction or the second direction. The implantation may be performed prior to or following trabeculotomy and/or the fluid delivery, and the trabeculotomy may be performed prior to, following, or simultaneously with the fluid delivery.

The acts performed as part of the methods herein may be ordered in any suitable way. Accordingly, various methods may be constructed in which acts are performed in an order different than illustrated, which may include performing some acts simultaneously, even though shown as sequential acts in illustrative examples.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

1. A system for treating an ocular condition, comprising:
an ocular implant; and
a delivery device comprising:
a cannula comprising a straight proximal portion and a curved distal portion, the curved distal portion comprising a first curve in a first direction; and
a positioning tool positioned at least partially within the cannula and configured to advance the ocular implant through the cannula, the positioning tool comprising a curved distal portion biased to form a second curve in a second, different direction.

2. The system of claim 1, wherein, when the positioning tool is positioned in the straight proximal portion of the cannula, the curved distal portion of the positioning tool comprises the second curve in the second direction.

3. The system of claim 1, wherein the second direction is opposite the first direction.

4. The system of claim 1, wherein the cannula comprises a top cross-sectional arc and a bottom cross-sectional arc, and wherein at least a portion of the curved distal portion of the positioning tool is configured to contact an inner surface of the top cross-sectional arc of the cannula during at least a portion of delivery of the ocular implant into Schlemm's canal.

5. The system of claim 1, wherein the cannula further comprises a plane longitudinally bisecting the straight proximal portion, and wherein the curved distal portion of the cannula curves toward a first side of the plane and the curved distal portion of the positioning tool curves away from the first side of the plane.

6. The system of claim 1, wherein the cannula further comprises a YZ-plane longitudinally bisecting the straight proximal portion, and wherein at least a portion of the curved distal portion of the cannula is on a first side of the YZ-plane and at least a portion of the curved distal portion of the positioning tool is on a second side of the YZ-plane.

7. The system of claim 1, wherein the first curve comprises a first radius of curvature (ROC) and the second curve comprises a second, greater ROC.

8. (canceled)

9. The system of claim 1, wherein the positioning tool further comprises a skived portion configured to increase flexibility of the positioning tool.

10. The system of claim 9, wherein the skived portion is within the curved distal portion of the positioning tool.

11. The system of claim 9, wherein the skived portion extends longitudinally along a side of the positioning tool and faces the first direction.

12. The system of claim 9, wherein the skived portion comprises a length of about 1 mm to about 8 mm.

13. The system of claim 9, wherein the skived portion comprises a depth of about 50 μm to about 150 μm.

14. (canceled)

15. The system of claim 14, wherein the second curve of the positioning tool is configured to cause at least a portion of the ocular implant to contact an interior surface of the cannula during advancement of the ocular implant through the cannula.

16. The system of claim 1, wherein the cannula comprises a blunt distal tip.

17. A system for treating an ocular condition, comprising:
an ocular implant; and
a delivery device comprising:
a cannula comprising a curved distal portion, the curved distal portion comprising a proximal portion and a distal portion defining a first radius of curvature (ROC) therebetween; and
a positioning tool positioned at least partially within the cannula and configured to advance the ocular implant through the cannula, the positioning tool comprising a curved distal portion comprising a proximal portion and a distal portion defining a second radius of curvature (ROC) therebetween, wherein the second ROC is greater than the first ROC.

18. The system of claim 17, wherein the curved distal portion of the cannula comprises a first curve having a first direction of curvature and the curved distal portion of the positioning tool comprises a second curve having a second direction of curvature counter to the first direction of curvature.

19. The system of claim 18, wherein the curved distal portion of the positioning tool is biased to form the second curve.

20. A system for treating an ocular condition, comprising:
an ocular implant; and
a delivery device comprising:
a cannula comprising a top cross-sectional arc and a bottom cross-sectional arc; and
a positioning tool positioned at least partially within the cannula and comprising a curved distal portion, wherein the positioning tool is configured to advance the ocular implant through the cannula, and wherein at least a portion of the curved distal portion of the positioning tool is configured to contact an inner surface of the top cross-sectional arc of the cannula during at least a portion of a procedure for delivering the ocular implant into Schlemm's canal.

21. The system of claim 20, wherein the cannula further comprises a curved distal portion comprising a first curve in a first direction, and the positioning tool comprises a curved distal portion biased to form a second curve in a second, different direction.

22.-70. (canceled)

71. The system of claim 1, wherein the ocular implant comprises a body implantable circumferentially within at least a portion of Schlemm's canal, the body comprising:
a twisted portion configured to restore or maintain at least partial patency of at least the portion of Schlemm's canal;
a proximal portion extending from the twisted portion and configured to releasably couple to the positioning tool of the delivery device; and
a distal portion extending from the twisted portion, wherein the distal portion is non-twisted.

72. The system of claim 71, wherein the twisted portion comprises a first twisted segment and a second twisted segment, and wherein the first twisted segment comprises a smaller radius of curvature (ROC) than the second twisted segment.

73. The system of claim 71, wherein the proximal portion comprises a hook configured to releasably couple with an opening extending transversely through a distal portion of the positioning tool.

74. The system of claim 71, wherein the proximal portion is configured to automatically release from the positioning tool during delivery of the ocular implant to Schlemm's canal.
